(19) United States
(12) Patent Application Publication
(10) Pub. No.: US 2024/0208969 A1
(43) Pub. Date: Jun. 27, 2024

(54) SUBSTITUTED FUSED BICYCLIC COMPOUNDS AS PARP INHIBITORS AND THE USE THEREOF

(71) Applicant: IMPACT THERAPEUTICS (SHANGHAI), INC., Shanghai (CN)

(72) Inventors: Sui Xiong CAI, Shanghai (CN); Ye Edward TIAN, Shanghai (CN); Xiaozhu WANG, Jiangsu (CN)

(21) Appl. No.: 18/554,980

(22) PCT Filed: Apr. 12, 2022
(86) PCT No.: PCT/CN2022/086311

§ 371 Date: Oct. 11, 2023

(30) Foreign Application Priority Data
Apr. 12, 2021 (CN) 202110392892.4
Jun. 24, 2021 (CN) 202110707739.6
Sep. 10, 2021 (CN) 202111065912.3

(51) Int. Cl. C07D 471/04; A61K 45/06; A61P 35/00; C07D 239/80; C07D 239/96; C07D 401/12; C07D 401/14; C07D 403/12; C07D 413/14

(52) U.S. Cl. CPC C07D 471/04; A61K 45/06; A61P 35/00; C07D 239/80; C07D 239/96; C07D 401/12; C07D 401/14; C07D 403/12; C07D 413/14

(57) ABSTRACT

The disclosure provides substituted fused bicyclic compounds as PARP inhibitors and the the use thereof. This disclosure provides compounds represented by Formula I as below, wherein A₁. A₂, A₃, R₁, L, Cy and n are defined herein. The compounds of Formula I of the present disclosure are PARP inhibitors and thus are useful in the treatment of diseases, disorders and conditions, such as cancer. responsive to the inhibition of PARP activity. The present disclosure also relates to a pharmaceutical composition comprising the compound of Formula I and the use of the compound of Formula I in the preparation of a medicament for the treatment or prevention of diseases or conditions responsive to the inhibition of PARP activity.

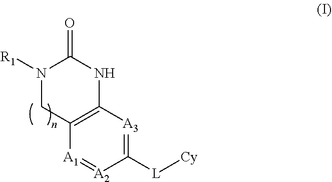

### FIELD OF THE DISCLOSURE

This disclosure is in the field of medicinal chemistry. In particular, the disclosure relates to substituted fused bicyclic compounds, and the use of these compounds as therapeutically effective PARP inhibitors and anticancer drugs.

### BACKGROUND OF THE INVENTION

Poly (ADP-ribose) polymerase (PARP) is a family of proteins, which transfer negatively charged ADP-ribose groups from donor NAD⁺ onto target proteins. That is one of many posttranscriptional modifications. Therefore, PARP also is termed ADP-ribose transferase.

Humans are thought to express 17 PARPs identified based on amino acid sequence homology to the catalytic domain (Vyas et al., 2013 Nature Communication, 4, 3240/1-3240/13). PARPs either catalyze the addition of a single ADP-ribose unit on target proteins or catalyze the polymerization of ADP-ribose units to form poly ADP-ribose, also known as Poly (ADP-Ribose) modification. As a result, the PARP family is further grouped into two subfamilies accordingly. Post-translational modification of poly (ADP-ribose) regulate many aspects of protein function and the physiological function of many PARPs have not been established.

The most characteristic member of the PARP family is PARP1, which was found to have the highest intracellular levels. PARP1 consists of 1014 amino acids (NCBI Accession P09874) long with a total molecular weight of approximately 116 kDa. Structurally, this enzyme is composed of two main domains including an N-terminal DNA-binding domain and a catalytic domain. PARP1 is known to play an important role in many cellular functions, including gene expression, transcription, cell division, cell differentiation, cell apoptosis, DNA damage response and repair. PARP1 is activated when DNA damage occurs and is involved in base excision repair (BER) which is a major mechanism of DNA single-strand damage repair. PARP1 binds to the site of Single Strand Break (SSB), and subsequently repair DNA via BER. In response to DNA damage, cells also have evolved two main repair pathways: Homologous Recombination (HR) and Non-Homologous End Joining (NHEJ), in addition to BER repair mechanisms. PARP inhibitors have shown to be sensitive to HR deficient tumors, indicating homologous recombination defects and PARP1 inhibition formed a pair of Synthetic Lethality, which has been validated by clinical trials. Several PARP inhibitors are currently approved for the treatment of breast, ovarian, pancreatic and prostate cancers with BRCA1/2 mutation.

PARP2 is a protein of 559 amino acids with molecular masses approximately 62 kDa and composed of DNA binding domain and catalytic regions domain (Ame et al., 1999 J Biol Chem 274:17860). The catalytic domain of PARP2 is very similar to that of PARP1. PARP2 is also proved to have similar functions to PARP1 and is involved in the repair of DNA damage repair through BER mechanism (Schreiber et al., 2002 J Biol Chem 277:23028). PARP inhibitors on the market, such as Olaparib, Niraparib, Talazoparib and Rucaparib, not only have inhibitory activities against PARP1, but also have similar inhibitory activities against PARP2. Based on the results of clinical trials, the pharmacodynamic effects of these PARP inhibitors on the market are comparable whereas their toxicity profiles are quite different. For example, these PARP inhibitors have similar hematological toxicity, but Talazoparib has similar side effects to chemotherapy drugs such as hair loss. Talazoparib also shows more potent inhibitory activity against TNKS1/2 than other PARP inhibitors (PARPi) in biochemical assay (Ryan et al., 2021, J Biol Chem 296:100251). Tankyrase 1(TNKS1) and Tankyrase 1(TNKS2) share 83% sequence identity overall, and their catalytic domain sequences are 89% identical. They play roles in DNA repair, telomere maintenance, and Wnt/β-catenin signaling. Targeting other PARPs except PARP1 may be the reason why PARP inhibitors cause exogenous toxicity, such as hair loss and diarrhea. In addition, inhibition of PARP2 activity has been proved may lead to hematotoxicity (Farés et al., 2013, Blood 122:44; Farés et al., 2015, Cep Death and Differentiation 22:1144). The toxicity of these PARP inhibitors limits their clinical application and combination with other targeted drugs.

Therefore, to improve, enhance and expand the clinical application of PARP1 inhibitors, it is necessary to explore highly selective PARP1 inhibitor with reduced toxicity, whether mechanism related or mechanism—independent.

Various PARP1 inhibitors have been disclosed. For example, WO2011006803, WO2013014038, WO2021013735 and WO2021260092.

### SUMMARY OF THE DISCLOSURE

The disclosure provides compounds and analogues as represented in Formulae 1, II, III and IV, the compounds can be used as PARP inhibitors. In particular, the compounds of the disclosure are selective PARP1 inhibitors relative to PARP2.

The disclosure also provides pharmaceutical compositions comprising an effective amount of the compound of Formulae I, II, III and IV for the treatment of cancer.

In a specific embodiment, the pharmaceutical composition may also contain one or more pharmaceutically acceptable carriers or diluents, for the treatment of cancer.

In a specific embodiment, the pharmaceutical composition may also contain at least one known anticancer drug or pharmaceutically acceptable salts thereof, for the treatment of cancer.

The disclosure is also directed to methods for the preparation of novel compounds of Formulae I, II, III and IV.

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

The term “hydrogen (H)” as employed herein includes its isotopes D and T.

The term “alkyl” as used herein refers to alkyl itself or a straight or branched chain radical of up to ten carbons. Useful alkyl groups include straight-chain or branched C₁₋₁₀ alkyl groups, preferably C₁₋₆ alkyl groups. In some embodiments, alkyl is C₁₋₄ alkyl. In some embodiments, alkyl is C₁₋₃ alkyl. Typical C₁₋₁₀ alkyl groups include methyl, ethyl, propyl, isopropyl, butyl, sec-butyl, tert-butyl, 3-pentyl, hexyl and octyl groups, which may he optionally substituted.

The term “alkenyl” as used herein refers to a straight or branched chain radical of 2-10 carbon atoms, unless the chain length is limited thereto, wherein there is at least one double bond between two of the carbon atoms in the chain; preferably, C₂₋₆ alkenyl. Typical alkenyl groups include ethenyl, 1-propenyl, 2-propenyl, 2-methyl-1-propenyl, 1-butenyl and 2- butenyl.

The term “alkynyl” as used herein refers to a straight or branched chain radical of 2-10 carbon atoms, unless the chain length is limited thereto, wherein there is at least one triple bond between two of the carbon atoms in the chain; C₂₋₆ preferably, alkynyl. Typical alkynyl groups include ethenyl, 1-propenyl, 1-methyl-2-propenyl, 2-propenyl, 1-butenyl and 2-butenyl.

Useful alkoxy groups include oxygen substituted by the above mentioned C₁₋₁₀ alkyl groups, preferred C₁₋₆ alkyl groups or C₁₋₄ alkyl groups, e.g., methoxy, ethoxy, etc. The alkyl in the alkoxy groups may be optionally substituted. Substituents of alkoxy groups include, without limitation, halogen, morpholino, amino (including alkylamino and dialkylamino), and carboxy (including esters thereof).

Useful amino and optionally substituted amino groups are —NR′R″, wherein R′ and R″ each are independently hydrogen, an optionally substituted C₁₋₁₀ alkyl, an optionally substituted C₃₋₈ cycloalkyl, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl, or R′ and R″ together with the N to which they are attached form an optionally substituted 4-7 membered cyclic amino group, which optionally comprises one or more (such as 2, 3) additional heteroatoms selected from a group consisting of O, N and S. Preferred amino groups include NH₂, and at least one of R′ and R″ is a C₁₋₆ alkyl in —NR′R″.

The term “oxo” as used herein refers to ═O.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆₋₁₄ aryl groups, preferably C₆₋₁₀ aryl groups. Typical C₆₋₁₄ aryl groups include phenyl, naphthyl, phenanthryl, anthracyl, indenyl, azulyl, biphenyl, biphenylene and fluorenyl.

The term “carbocyclic group” as used herein include cycloalkyl and partially saturated carbocyclic groups. Useful cycloalkyl groups are C₃₋₈ cycloalkyl. In some preferred embodiments, cycloalkyl groups are C₃₋₆ cycloalkyl. Typical cycloalkyl groups include cyclopropyl, cyclobutyl, cyclopentyl, cyclohexyl and cycloheptyl. Useful partially saturated carbocyclic groups are cycloalkenyl, such as C₃₋₈ cycloalkenyl, which include cyclopentenyl, cycloheptenyl and cyclooctenyl. Carbocyclic group may be substituted by one or more substituents as described herein.

Useful halo or halogen groups include fluoro, chloro, bromo and iodo.

Useful acylamino (acylamido) groups are any C₁₋₆ acyl (alkanoyl) attached to an amino nitrogen, e.g., acetamino, propionamido, butanoylamido, pentanoylamido and hexanoylamido, as well as aryl-substituted C₁₋₆ acylamino groups, e.g., benzoylamido.

Useful acyl groups include C₁₋₆ acyl groups, such as acetyl. Acyl may be optionally substituted by group selected from a group consisting of halo, amino and aryl, wherein the amino and an may be optionally substituted. When acyl is substituted by halo, the number of halogen substituents may be in the range of 1-5. Examples of substituted acyls include chloroacetyl and pentafluorobenzoyl. When acyl is substituted by amino, amino group may be substituted by one or two substituents as described herein. In some embodiments, aminoacyl is —C(O)—NR R,′R″, wherein R′ and R″ each are independently hydrogen, an optionally substituted C₁₋₁₀ alkyl, an optionally substituted C₃₋₈ cycloalkyl, an optionally substituted aryl or an optionally substituted heteroaryl. Preferably, R′ and R″ each are independently hydrogen, an optionally substituted C₁₋₄ alkyl, or an optionally substituted C₃₋₆ cycloalkyl. Herein, when the alkyl, cycloalkyl, aryl and heteroaryl groups in the R′ and R″ are substituted, the substituents are as described in any of the embodiments herein, and preferred substituents include halogen, hydroxyl, amino and alkyl, etc.

The term “heterocyclic group” as used herein refers to a saturated or partially saturated 3-7 membered monocyclic, or 7-10 membered bicyclic ring, spirocyclic ring or bridged ring system, which consists of carbon atoms and one to four heteroatoms independently selected from a group consisting of O, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocycle can be substituted on carbon atom or nitrogen atom if the resulting compound is stable. Heterocyclic group may be substituted by one or more substituents as described herein.

Useful saturated or partially saturated heterocyclic groups include tetrahydrofuranyl, tetrahydropyranyl, piperidinyl, piperazinyl, 1,4-diazepanyl, azetidinyl, oxetanyl, pyrrolidinyl, imidazolidinyl, imidazolinyl, indoline, isoindoline, quinuclidinyl, morpholinyl, isochromanyl, chromanyl, pyrazolidine, pyrazolinyl, tetrahydroisoquinolyl, tetronoyl and tetramoyl, which may be optionally substituted by one or more substituents as described herein.

The term “heteroaryl” as used herein refers to a group having 5 to 14 ring atoms, preferably 5 to 10 ring atoms, with 6, 10 or 14 electrons shared in a cyclic array. Ring atoms are carbon atoms and 1-3 heteroatoms selected from a group consisting of oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl, and 4-pyridyl), pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, tetrahydrocyclopenta[c]pyrazol-3-yl, benzoisoxazolyl such as 1,2-benzoisoxazol-3-yl, benzimidazolyl, 2-oxindolyl, thiadiazolyl, 2-oxobenzimidazolyl, imidazopyridazinyl, imidazopyridyl, triazolopyridazinyl, pyrazolopyrimidinyl, pyrrolopyrimidinyl, pyrrolopyridyl, pyrrolopyrazinyl or triazolopyrazinyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

In this disclosure, unless otherwise described, when substituted, the alkyl, cycloalkyl, heterocycloalkyl, alkoxy, heterocycloalkoxy, alkenyl, heterocycloalkenyl, alkynyl, amino, amido, acyloxy, carboxyl, hydroxyl, mercapto, alkylthio sulfonyl, sulfonyl, sulfinyl, aminoacyl, silyl, phosphinecarboxy, phosphono, carbocyclic group, heterocyclic group, aryl or heteroaryl as described in any embodiment herein may be substituted by one or more (such as 1, 2, 3, 4, 5 or 6) substituents selected from a group consisting of the group consisting of halogen, hydroxyl, carboxyl, amino, nitro, cyano, C₁₋₆ amido, C₁₋₆ acyloxy, C₁₋₆ alkoxy, aryloxy, alkylthio, C₁₋₆ alkyl, C₁₋₆ acyl, C₆₋₁₀ aryl, C₃₋₈ cycloalkyl, C₂₋₆ alkenyl, C₂₋₆ alkynyl, heterocyclic or heteroaryl, methylenedioxy, urea group, mercapto group, azide group, carbonyl, alkanesulfonyl, sulfamoyl, dialkylsulfamoyl and alkylsulfinyl, etc. The substituent itself may also be optionally substituted. Preferred substituents include without limitation halogen, hydroxyl, carboxyl, amino, C₁₋₆ amido, C₁₋₆ acyloxy, C₁₋₆ alkoxy, C₁₋₆ alkyl, C₁₋₆ acyl and alkanesulfonyl.

It should be understood that in each embodiment, when the substituent is a heterocyclic group, aryl or heteroaryl, the number thereof is usually 1.

Specifically, the disclosure provides compounds represented by Formula I:

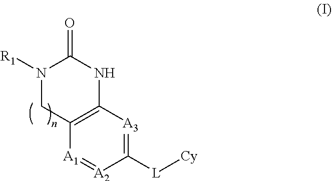

or stereoisomers, tautomers, N-oxides, hydrates, solvates, isotope-substituted derivatives, or pharmaceutically acceptable salts thereof, or mixtures thereof, or prodrugs thereof, wherein:
R₁ is selected from a group consisting of an optionally substituted alkyl, an optionally substituted carbocyclic group, an optionally substituted alkenyl and an optionally substituted alkynyl;
A₁, A₂ and A₃ are each independently selected from a group consisting of N and CR₂;
L is selected from a group consisting of a bond and an alkylene optionally substituted by R₃ and/or R₄;
Cy is selected from a group consisting of an optionally substituted heterocyclic group, an optionally substituted aryl, and an optionally substituted heteroaryl;
R₂ is selected from a group consisting of hydrogen, halogen, an optionally substituted alkyl, an optionally substituted alkoxy and an optionally substituted carbocyclic group;
R₃ and R₄ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₃ and R₄ together with the attached C form a ring;
n is selected from a group consisting of 0, 1 and 2;
wherein when n is 1 or 2, the indicated —(CH₂)ₙ— is optionally substituted by a ═O.

In Formula I and each formula of the disclosure, unless otherwise described, each alkyl is independently a C₁₋₆ alkyl, preferably a C₁₋₄ alkyl; each alkylene is a C₁₋₆ alkylene, preferably a C₁₋₃ alkylene; each alkenyl is independently a C₂₋₆ alkenyl, preferably C₂₋₄ alkenyl; each alkynyl is independently C₂₋₆ alkynyl, preferably C₂₋₄ alkynyl; each alkoxy is independently C₁₋₆ alkoxy, preferably C₁₋₄ alkoxy. Preferably, when the alkyl, alkenyl, alkynyl, and alkoxy are substituted, the substituents can be selected from a group consisting of cyano, hydroxyl, nitro, amino(—NR′R″), aryl, heterocyclic group, heteroaryl, halogen and carboxyl, etc. The number of substituents may be 1-5, R′ and R″ are preferably each independently H, an optionally substituted C₁₋₄ alkyl or an optionally substituted C₃₋₆ cycloalkyl. For example, the substituted alkyl per se or as substituents of other groups may be hydroxyalkyl, dihydroxyalkyl, alkylaminoalkyl, dialkylaminoalkyl, heterocyclicalkyl, aralkyl, heteroarylalkyl and haloalkyl, etc. It should be understood, when the substituent is aryl, heteroaryl, heterocyclic group, cyano, nitro and carboxyl, the number thereof is usually 1. When the substituent is halogen, the number of substituents can be up to 5 depending on the carbon chain length of the alkyl, alkenyl, alkynyl and alkoxy groups; exemplary substituents are trifluoromethyl and pentafluoroethyl, etc.

In Formula I and each formula of the disclosure, unless otherwise described, the number of ring carbon atoms of each carbocyclic group is preferably 3-8. Preferred carbocyclic groups are C₃₋₈ cycloalkyl groups. The substituents on the carbocyclic group are preferably C₁₋₄ alkyl, halogenated C₁₋₄ alkyl, C₁₋₄ alkoxy, halogen, hydroxyl, carboxyl, amino (—NR′R″), aryl, heterocyclic group, heteroaryl and carboxyl, etc. The number of substituents may be 1-5. R′ and R″ are preferably each independently H, an optionally substituted C₁₋₄ alkyl or an optionally substituted C₃₋₆ cycloalkyl. It should be understood, when the substituent is aryl, heteroaryl, heterocyclic group, cyano, nitro and carboxyl, the number thereof is usually 1. When the substituent is halogen, the number of substituents can be up to 5. The aryl, the heterocyclic group and the heteroaryl, as a substituent of the carbocyclic group, may be optionally substituted as described herein.

In Formula I and each formula of the disclosure, unless otherwise described, the aryl refers to C₆₋₁₄ aryl, the heteroaryl refers to 5-10 membered heteroaryl, and the heterocyclic group refers to 4-10 membered heterocyclic group. In some embodiments, the substituents on each of the aryl, heteroaryl and heterocyclic group can be independently selected from a group consisting of C₁₋₄ alkyl, halogenated C_{1-4 ,} alkyl, C₁₋₄ alkoxy, halogen, hydroxyl, carboxyl, amino (—NR′R″), —S(O)₂—NR′R″ an optionally substituted aryl, an optionally substituted heteroaryl, an optionally substituted heterocyclic group, halogen, amido, aminoacyl (—C(O)—NR′R″) and carboxyl, etc.; wherein R′ and R″ each are independently hydrogen, an optionally substituted C₁₋₁₀ alkyl, an optionally substituted C₃₋₈ cycloalkyl, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl. The number of substituents may be 1-5. In some embodiments, the said optionally substituted aryl, optionally substituted heteroaryl and optionally substituted heterocyclic group may be optionally substituted by 1-5 groups selected from a group consisting of C₁₋₄ alkyl, halogenated C₁₋₄ alkyl, C₁₋₄ alkoxy, halogen, hydroxyl, carboxyl, amino (—NR′R″), —S(O)₂—NR′R″, aminoacyl (—C(O)—NR′R″) and carboxyl, wherein the said R′ and R″ are preferably each independently H, an optionally substituted C₁₋₄ alkyl or an optionally substituted C₃₋₆ cycloalkyl. It should be understood, when the substituent is aryl, heteroaryl, heterocyclic group, cyano, nitro and carboxyl, the number thereof is usually 1. When the substituent is halogen, the number of substituents can be up to 5.

In one or more embodiments of the compound of Formula I, the said aryl is preferably a phenyl. The said heteroaryl is a 5-1.0-membered heteroaryl containing 1 or 2 nitrogen atoms, including but is not limited to pyridyl, pyrazinyl, pyrrolyl, imidazolyl, pyrazolyl, pyrimidinyl, pyridazinyl, indolyl, pyridopyrimidinyl, indolyl, indazolyl and benzimidazolyl, etc. The said carbocyclic group is preferably a C₃₋₈ cycloalkyl. The said heterocyclic group is preferably a 4-10 membered heterocyclic group containing O and/or N, including but not limited to azetidinyl, oxetanyl, pyrrolidinyl, piperazinyl, piperidinyl, tetrahydrofuranyl, tetrahydroisoquinolyl and morpholinyl, etc.

In one or more embodiments of the compound of Formula I, when R₂ is substituted, the substituents can be 1-5 groups selected from a group consisting of halogen and hydroxyl. Preferably, R₂ is hydrogen, halogen, an optionally substituted C₁₋₃ alkyl or an optionally substituted C₁₋₃ alkoxy; more preferably, R₂ is hydrogen, C₁₋₃ alkyl or halogen. In some preferred embodiments, only one of A₁, A₂ and A₃ is N, and the other two are independently CR₂, preferably, R₂ is independently H, C₁₋₃ alkyl or halogen. In more preferred embodiment, A₃ is CH, one of A₁ and A₂ is N and the other is CR₂, wherein R₂ is H, C₁₋₃ alkyl or halogen. In some embodiments, A₁ is N, and both of A₂ and A₃ are CH. In some embodiments, A₂ is N and both A₁ and A₃ are CH. In some embodiments, all of A₁, A₂ and A₃ are CR₂, each R₂ is independently H, C₁₋₃ alkyl or halogen. Preferably, A₃ is CH, one of A₁ and A₂ is CR₂, wherein R₂ is H, C₁₋₃ alkyl or halogen; more preferably, both of A₂ and A₃ are CH, A₁ is CR₂, wherein R₂ is C₁₋₃ alkyl or halogen.

In one or more embodiments of the compound of Formula I, R₁ is an optionally substituted C₁₋₃ alkyl or an optionally substituted C₃₋₆ cycloalkyl. Preferably, when R₁ is substituted, the substituents can be 1-5 groups selected from a group consisting of halogen, hydroxyl, amino (—NR′R″), etc.; wherein R′ and R″ are preferably each independently H, or C₁₋₄ alkyl or C₃₋₆ cycloalkyl optionally substituted by 1-5 groups selected from a group consisting of hydroxyl and halogen. Preferably, R₁ is C₁₋₃ alkyl, halogenated C₁₋₃ alkyl or C₃-1 cycloalkyl.

In one or more embodiments of the compound of Formula I, preferably, R₃ and R₄ are each independently halogen or C₁₋₃ alkyl.

In one or more embodiments of the compound of Formula I, L is bond. In some embodiments, L is an unsubstituted alkylene, more preferably an unsubstituted C₁₋₃ alkylene, preferably methylene.

In one or more embodiments of the compound of Formula I, Cy may be substituted by 1-5, preferably 1-3 groups selected from a group consisting of halogen, C₁₋₄ alkyl, C₁₋₄ alkoxy, halogenated C₁₋₄ alkyl, halogenated C₁₋₄ alkoxy, an optionally substituted 6-14 membered aryl, an optionally substituted 5-10 membered heteroaryl, an optionally substituted 4-10 membered heterocyclic group, an optionally substituted C₃₋₈ cycloalkyl and —(CH₂)ₘC(O)—NRₐR_{b}; wherein Rₐ and R_{b} can be independently H, C₁₋₄ alkyl, an optionally substituted 6-14 membered aryl, an optionally substituted 5-10 membered heteroaryl or an optionally substituted 4-10 membered heterocyclic group, m is an integer from 0 to 5, preferably, m is 0, preferably, at least one of Rₐ and R_{b} is an optionally substituted 6-14 membered aryl, an optionally substituted 5-10 membered heteroaryl or an optionally substituted 4-10 membered heterocyclic group. The said optionally substituted 6-14 membered aryl, optionally substituted 5-10 membered heteroaryl, optionally substituted 4-10 membered heterocyclic group and optionally substituted C₃₋₈ cycloalkyl in the definition of Cy substituents and optionally substituted 6-14 membered aryl, optionally substituted 5-10 membered heteroaryl or optionally substituted 4-10 membered heterocyclic group in the definitions of Rₐ and R_{b} can each independently be substituted by 1-5 groups selected from a group consisting of halogen, C₁₋₄ alkyl, C₁₋₄ alkoxy, halogenated C₁₋₄ alkyl, halogenated C₁₋₄ alkoxy, an optionally substituted 5-10 membered heteroaryl (such as optionally substituted by 1-3 substituents selected from a group consisting of halogen and C₁₋₄ alkyl, preferably 5-6 membered heteroaryl containing nitrogen and/or oxygen), —S(O)₂NR′R″, —NR′R″ and —C(O)—NR′R″, wherein the said R′ and R″ are preferably each independently H, an optionally substituted C₁₋₄ alkyl or an optionally substituted C₃₋₆ cycloalkyl. More preferably, R′ and R″ are each independently H, C₁₋₄ alkyl, halogenated C₁₋₄ alkyl or C₃₋₆ cycloalkyl. In some preferred embodiments, the substituents on the optionally substituted 5-10 membered heteroaryl, optionally substituted 4-10 membered heterocyclic group and optionally substituted C₃₋₈ cycloalkyl in the definition of Cy and the optionally substituted 6-14 membered aryl, optionally substituted 5-10 membered heteroaryl and optionally substituted 4-10 membered. heterocyclic group in the definition of Rₐ and R_{b} include at least —C(O)—NR′R″ and optionally further include any one or two of halogen, C₁₋₄ alkoxy and C₁₋₄ alkyl. In some preferred embodiments, Cy is substituted by an optionally substituted 5-10 membered heteroaryl, preferably by an optionally substituted 5-10 membered nitrogen-containing heteroaryl. Preferably, the 5-10 membered nitrogen-containing heteroaryl is at least substituted by —C(O)—NR′R″, and optionally by any one or two of halogen, C₁₋₄ alkoxy and C₁-4 alkyl. In some embodiments, Cy is substituted by one R₅ as described below. In some embodiments, a heterocyclic group optionally substituted as described above. In some particularly preferred embodiment, Cy is piperazinyl substituted with an optionally substituted pyridyl, and the said pyridyl is at least substituted with —C(O)—NR′R″. In other preferred embodiments, Cy is substituted by —(CH₂)ₘC(O)—NRₐR_{b}, preferably, at least one of Rₐ and R_{b} is a 5-10 membered heteroaryl substituted with —C(O)—NR′R″.

Preferably, in the embodiments as described herein, when the said R′ and R″ are substituted, the substituents are selected from a group consisting of halogen and hydroxy. In some preferred embodiments, R′ and R″ are preferably each independently H, C₁₋₄ alkyl, halogenated C₁₋₄ alkyl or C_{3 -6} cycloalkyl.

In one or more embodiments of the compound of Formula I, when L is a bond, Cy is an optionally substituted aryl or an optionally substituted heteroaryl. Preferably, the optionally substituted aryl and the optionally substituted heteroaryl are each optionally substituted by one or more groups selected from a group consisting of the consisting of C₁₋₄ alkyl, halogenated C₁₋₄ alkyl, C₁-4 alkoxy, halogen and —(CH₂)ₘC(O)—NRₐR_{b}, wherein Rₐ and R_{b} can be independently H, C₁₋₄ alkyl, an optionally substituted 6-14 membered aryl, an optionally substituted 5-10 membered heteroaryl or an optionally substituted 4-10 membered heterocyclic group, m is an integer from 0 to 5, preferably, m is 0. The number of substituents can be 1-5. Preferably, the optionally substituted aryl and optionally substituted heteroaryl are at least substituted by —(CH₂)ₘC(O)—NRₐR_{b}, and may be further substituted by 1-3 substituents selected from a group consisting of halogen, C₁₋₄ alkyl and halogenated C₁₋₄ alkyl. Preferably, at least one of Rₐ and R_{b} is all optionally substituted 6-14 membered aryl, an optionally substituted 5-10 membered heteroaryl or an optionally substituted 4-10 membered heterocyclic group. More preferably, one of Rₐ and R_{b} is H, the other is an optionally substituted 5-10 membered heteroaryl. The said optionally substituted 6-14 membered aryl, optionally substituted 5-10 membered heteroaryl and optionally substituted 4-10 membered heterocyclic group in the definitions of Rₐ and R_{b} may each be optionally substituted by 1-5 groups selected from a group consisting of C₁₋₄ alkyl, halogenated C₁₋₄ alkyl, C₁₋₄ alkoxy, halogen, hydroxyl, carboxyl, amino (—NR′R″), —C(O)—NR′R″ and carboxyl; wherein the said R′ and R″ are preferably each independently H, an optionally substituted C₁₋₄ alkyl or an optionally substituted C₃₋₆ cycloalkyl, more preferably, R′ and R″ are preferably each independently H, C₁₋₄ alkyl, halogenated C₁₋₄ alkyl or C₃₋₆ cycloalkyl. Further preferably, the 5-10 membered heteroaryl and the 4-10 membered heterocyclic group are at least substituted by —C(O)—NR′R″, and can be further substituted by 1-3 substituents selected from a group consisting of halogen, C₁₋₄ alkoxy, C₁₋₄ alkyl and halogenated C₁₋₄ alkyl.

In one or more embodiments of the compound of Formula I, when L is an alkylene group, such as —CH₂—, Cy is an optionally substituted 5-7 membered nitrogen-containing heterocyclic group. Preferably, the 5-7 membered nitrogen-containing heterocyclic group is covalently attached to L through its ring nitrogen atom. Further preferably, Cy is an optionally substituted piperazinyl. Preferably, the substituent on Cy is selected from a group consisting of halogen, C₁₋₄ alkyl, C₁₋₄ alkoxy, halogenated C₁₋₄ alkyl, halogenated C₁₋₄ alkoxy, an optionally substituted 6-14 membered aryl, an optionally substituted 5-10 membered heteroaryl, an optionally substituted 4-10 membered heterocyclic group and an optionally substituted C₃₋₈ cycloalkyl. The optionally substituted 6-14 membered aryl, the optionally substituted 5-10 membered heteroaryl, the optionally substituted 4-10 membered heterocyclic group and the optionally substituted C₃₋₈ cycloalkyl are each can be independently substituted by 1-5 members selected from a group consisting of halogen, C₁₋₄ alkyl, C₁₋₄ alkoxy, halogenated C₁₋₄ alkyl, halogenated C₁₋₄ alkoxy, optionally substituted 5-10 membered heteroaryl (such as optionally substituted by 1-3 substituents selected from a group consisting of halogen and C₁₋₄ alkyl, preferably 5-6 membered heteroaryl containing nitrogen and/or oxygen), —S(O)₂—NR′R″, —NR′R″ and —C(O)—NR′R″, wherein the said R′ and R″ are preferably each independently H, an optionally substituted C₁₋₄ alkyl or an optionally substituted C₃₋₆ cycloalkyl. In some preferred embodiments, the substituent(s) include at least —C(O)—NR′R″, and optionally include one or two of substituents selected from a group consisting of halogen, C₁₋₄ alkoxy and C₁₋₄ alkyl. In some preferred embodiments, Cy is substituted by an optionally substituted 5-10 membered heteroaryl, preferably by an optionally substituted 5-10 membered nitrogen-containing heteroaryl, preferably, the 5-10 membered nitrogen-containing heteroaryl is at least substituted by —C(O)—NR′R″ and optionally further substituted by one or two substituents selected from a group consisting of halogen, C₁₋₄ alkoxy and C₁₋₄ alkyl. In some particularly preferred embodiments, Cy is piperazinyl substituted with an optionally substituted pyridyl, and the said pyridyl is at least substituted with —C(O)—NR′R″. Preferably, in the embodiments as described herein, when the said R′ and R″ are substituted, the substituents are selected from a group consisting of halogen and hydroxy. In some preferred embodiments, R′ and R″ are preferably each independently H, C₁₋₄ alkyl, halogenated C₁₋₄ alkyl or C₃₋₆ cycloalkyl.

In one or more embodiments of the compound of Formula I, n is 0 or 1.

One group of preferred compounds of Formula I in this disclosure is represented by compounds of Formula II (including Formulae IIa and IIb):

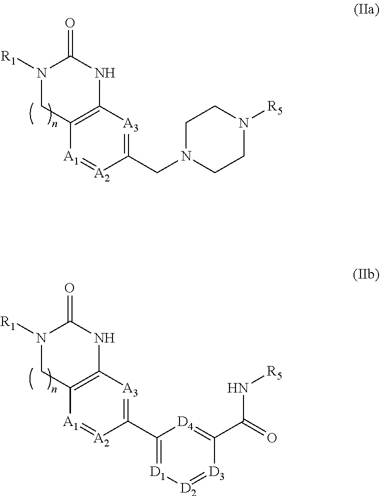

or stereoisomers, tautomers, N-oxides, hydrates, solvates, isotope-substituted derivatives, or pharmaceutically acceptable salts thereof, or mixtures thereof, or prodrugs thereof, wherein:
R₁, A₁, A₂, A₃ and n are as defined in Formula I;
R₅ is selected from a group consisting of an optionally substituted aryl and an optionally substituted heteroaryl;
D₁, D₂, D₃ and D₄ are independently selected from a group consisting of N and CR₆;
R₆ is selected from a group consisting of hydrogen, halogen, an optionally substituted alkyl, an optionally substituted alkoxy, an optionally substituted carbocyclic group, an optionally substituted alkenyl, and an optionally substituted alkynyl.

In one or more embodiments of the compound of Formula IIa and Formula IIb, R₁ is an optionally substituted C₁₋₃ alkyl or C₃₋₆ cycloalkyl. Preferably, when R₁ is substituted, the substituents can be 1-5 groups selected from a group consisting of halogen, hydroxyl, amino (—NR′R″), etc.; wherein R′ and R″ are preferably each independently H, an optionally substituted C₁₋₄ alkyl or an optionally substituted C₃₋₆ cycloalkyl. Preferably, R₁ is C₁₋₃ alkyl, halogenated C₁₋₃ alkyl or C₃₋₄ cycloalkyl.

In one or more embodiments of the compound of Formula IIa and Formula IIb, when R₂ is substituted, the substituents can be 1-5 groups selected from a group consisting of halogen and hydroxyl. Preferably, R₃ is hydrogen, halogen, an optionally substituted C₁₋₃ alkyl or an optionally substituted C₁₋₃ alkoxy; more preferably, R₂ is hydrogen, C₁₋₃ alkyl or halogen. In some preferred embodiments, only one of A₁, A₂ and A₃ is N, and the other two are independently CR₂, preferably, R₂ is independently H, C₁₋₃ alkyl or halogen. In more preferred embodiment, A₃ is CH, one of A₁ and A₂ is N and the other is CR₂, wherein R₂ is H, C₁₋₃ alkyl or halogen. In some embodiments, A₁ is N, and both A₂ and A₃ are CH. In some embodiments, A₂ is N and both of A₁ and A₃ are CH. In some embodiments, all of A_{1 ,} A₂ and A₃ are CR₂, each R₂ is independently H, C₁₋₃ alkyl or halogen. Preferably, A₃ is CH, one of A₁ and A₂ is CR₂, wherein R₂ is H, C₁₋₃ alkyl or halogen; more preferably, both of A₂ and A₃ are CH, A₁ is CR₂, wherein R₂ is C₁₋₃ alkyl or halogen.

In one or more embodiments of the compound of Formula IIa and Formula IIb, R₅ is an optionally substituted phenyl or a 5-7 membered nitrogen-containing heterocyclic group, more preferably an optionally substituted phenyl, pyridyl, pyridazinyl or pyrazinyl. Preferably, when R₅ is substituted, the substituents can be 1-5 groups selected from a group consisting of halogen, an optionally substituted alkyl, an optionally substituted alkoxy, an optionally substituted 5-10 membered heteroaryl (e.g. an optionally substituted with 1-3 substituents selected from a group consisting of halogen and C₁₋₄ alkyl, preferably 5-6-membered heteroaryl containing nitrogen and/or oxygen), —S(O)₂—NR′R″ and an optionally substituted aminoacyl. More preferably, R₅ is substituted in the para position with an optionally substituted aminoacyl group. Preferably, the optionally substituted aminoacyl is —C(O)—NR′R″, wherein the said R′ and R″ are preferably each independently H, an optionally substituted C₁₋₄ alkyl or an optionally substituted C₃₋₆ cycloalkyl. More preferably, R′ and R″ are preferably each independently H, C₁₋₄ alkyl, halogenated C₁₋₄ alkyl or C₃₋₆ cycloalkyl. In one or more embodiments of the compound of Formula IIa and Formula IIb, R₅ is 1-oxo-1,2,3,4-tetrahydroisoquinolin-6-yl or 4-oxo-4H-pyrido[1,2-a]pyrimidin-8-yl optionally substituted by 1-5 groups selected from a group consisting of halogen, C₁₋₄ alkyl, halogenated C₁₋₄ alkyl, C₁₋₄ alkoxy and halogenated C₁₋₄ alkoxy, etc. In one or more embodiments of the compound of Formula IIa and Formula IIb, R₅ is pyridopyrimidinyl, indolyl, indazolyl or benzimidazolyl optionally substituted by 1-3 groups selected from a group consisting of halogen, C₁₋₄ alkyl and halogenated C₁₋₄ alkyl.

In one or more embodiments of the compound of Formula IIa and Formula IIb, R₅ is preferably the following groups:

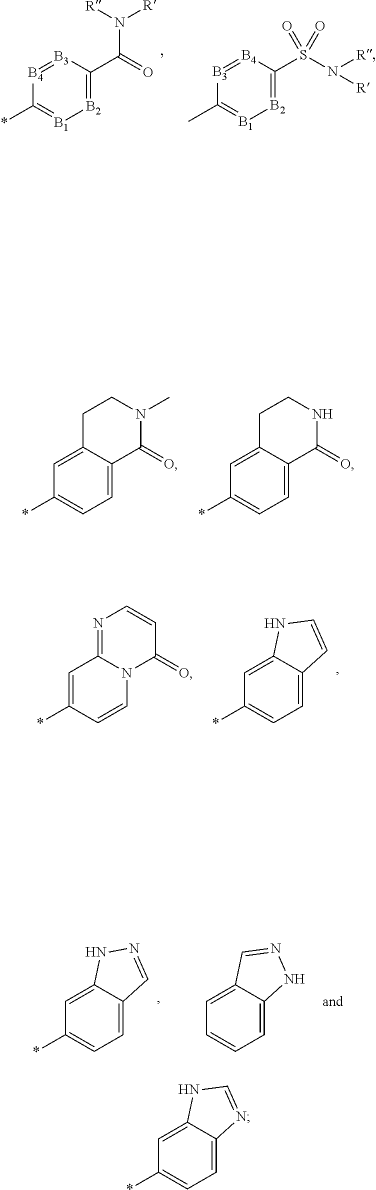

more preferably the following group:

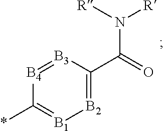

wherein B₁, B₂, B₃ and B₄ are independently selected from a group consisting of N and CR₇; R₇ is selected from a group consisting of hydrogen, halogen, C₁₋₄ alkyl, C₁₋₄ alkoxy, halogenated C₁₋₄ alkyl, halogenated C₁₋₄ alkoxy and —NR′R″; R′ and R″ are each independently selected from a group consisting of hydrogen, an optionally substituted C₁₋₁₀ alkyl, an optionally substituted C₃₋₈ cycloalkyl, an optionally substituted 6-14 membered aryl, or an optionally substituted 5-10 membered heteroaryl, preferably each independently are hydrogen, an optionally substituted C₁₋₄ alkyl or an optionally substituted C₃₋₆ cycloalkyl, more preferably each independently H, C₁₋₄ alkyl, halogenated C₁₋₄ alkyl or C₃₋₆ cycloalkyl; * indicates the position at which the group is attached to the rest of the compound. Preferably, the B₁-B₄ containing group is phenyl, pyridyl, pyrimidinyl or pyridazinyl. Preferably, R₇ is H, halogen, C₁₋₃ alkyl, C₁₋₃ alkoxy or halogenated C₁₋₃ alkyl. Preferably, B₃ is N, B₄ is CR₇, and B₁ and B₂ are CH, wherein R₇ is H, halogen, C₁₋₃ alkyl, C₁₋₃ alkoxy or halogenated C₁₋₃ alkyl.

In one or more embodiments of the compound of Formula IIa and Formula IIb, D₁, D₂, D₃ and D₄ are independently selected from a group consisting of N and CR₆, wherein R₆ is preferably hydrogen, halogen, an optionally substituted C₁₋₃ alkyl or an optionally substituted C₁₋₃ alkoxy. Preferably, when R₆ is substituted, the substituent may be 1-5 groups selected from a group consisting of halogen and hydroxyl. More preferably, R₆ is hydrogen, C₁₋₃ alkyl or halogen. In some preferred embodiments, the group containing D₁-D₄ is phenyl or pyridyl, which is optionally substituted by 1-3 groups selected from a group consisting of halogen and C₁₋₃ alkyl.

In one or more embodiments of the compound of Formula IIa and Formula IIb, n is 0 or 1.

In one or more embodiments of the compound of Formula IIa, R₁ is selected from a group consisting of the said optionally substituted alkyl, optionally substituted carbocyclic group, optionally substituted alkenyl and optionally substituted alkynyl; A₁, A₂ and A₃ are each independently selected from a group consisting of N and CR₂; R₅ is:

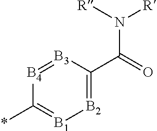

wherein B₁, B₂, B₃ and B₄ are independently selected from a group consisting of N and CR₇; R′ is H; R″ is selected from a group consisting of hydrogen, an optionally substituted C₁₋₁₀ alkyl, or an optionally substituted C₃₋₈ cycloalkyl; R₂ is selected from a group consisting of hydrogen, halogen, the said optionally substituted C₁₋₁₀ alkyl, optionally substituted C₁₋₁₀ alkoxy and optionally substituted C₃₋₈ cycloalkyl; R₇ is selected from a group consisting of hydrogen, halogen, C₁₋₄ alkyl, C₁₋₄ alkoxy, halogenated C₁₋₄ alkyl, halogenated C₁₋₄ alkoxy and —NR′R″, R′ and R″ each independently is H, C₁₋₄ alkyl, halogenated C₁₋₄ alkyl or C₃₋₆ cycloalkyl; n is 0, 1 or 2; wherein when n is 1 or 2, the —(CH₂)ₙ— is optionally substituted by ═O. Preferably, A₁, A₂ and A₃ are each N and CR₂, wherein R₂ is preferably hydrogen, halogen, an optionally substituted C₁₋₃ alkyl or an optionally substituted C₁₋₃ alkoxy, more preferably R₂ is hydrogen, C₁₋₃ alkyl or halogen. Preferably, only one of A₁, A₂ and A₃ is N, and the other two are independently CR₂, preferably, R₂ is independently H, C₁₋₃ alkyl or halogen. Preferably, A₃ is CH, one of A1 and A₂ is N and the other is CR₂, wherein R₂ is H, C₁₋₃ alkyl or halogen. Preferably, A₁ is N, and both of A₂ and A₃ are CH. In some embodiments, A₂ is N and both of A₁ and A₃ are CH. In some embodiments, all of A₁, A₂ and A₃ are CR₂, and each R₂ is independently H, C₁₋₃ alkyl or halogen; preferably, A₃ is CH, and one of A₁ and A₂ is CR₂, wherein R₂ is H, C₁₋₃ alkyl or halogen; more preferably, both of A₂ and A₃ are CH, A₁ is CR₂, and R₂ is C₁₋₃ alkyl or halogen. Preferably, B₁, B₂, B₃ and B₄ are each independently selected from a group consisting of N and CR₇, wherein R₇ is hydrogen, C₁₋₃ alkyl, halogenated C₁₋₃ alkyl or halogen. Preferably, both of B₁ and B₇, are CH, B₃ is N, and B₄ is CR₇, wherein R₇ is hydrogen, C₁₋₃ alkyl, halogenated C₁₋₃ alkyl or halogen. Preferably, R₁ is optionally substituted C₁₋₃ alkyl. Preferably, when R₁ is substituted, the number of substituents can be 1-5, which can be selected from a group consisting of halogen, hydroxyl, amino —NR′R″), etc.; wherein R′ and R″ are preferably each independently H, an optionally substituted alkyl or an optionally substituted C₃₋₆ cycloalkyl. Preferably, R₁ is C₁₋₃ alkyl or halogenated C₁₋₃ alkyl. Preferably, R′ and R″ are each independently hydrogen, an optionally substituted C₁₋₄ alkyl or an optionally substituted C₃₋₆ cycloalkyl. Preferably, when R′ and R″ are substituted, the number of substituents can be 1-5, which can be selected from a group consisting of halogen, hydroxyl, amino, etc. Preferably, R′ is hydrogen; R″ is hydrogen, C₁₋₄ alkyl, halogenated C₁₋₄ alkyl or C₃₋₆ cycloalkyl. Preferably, R₇ is hydrogen, halogen, C₁₋₃ alkyl or halogenated C₁₋₃ alkyl. Preferably, n is 0 or 1.

One group of preferred compounds of Formula I in this disclosure is represented by compounds of Formula III (including Formulae IIIa and IIIb):

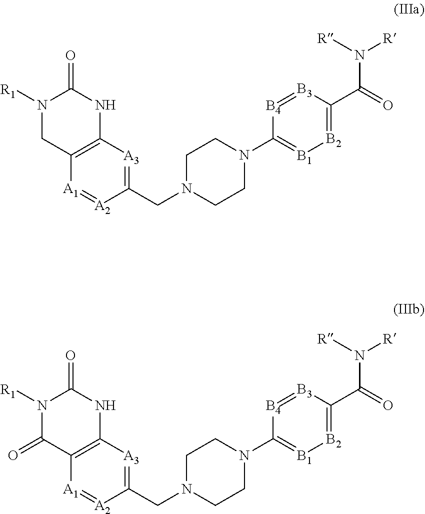

or stereoisomers, tautomers, N-oxides, hydrates, solvates, isotope-substituted derivatives, or pharmaceutically acceptable salts thereof, or mixtures thereof, or prodrugs thereof, wherein:
R₁, A₁, A₂, A₃, B₁, B₂, B₃ and B₄ are as described in any of the foregoing embodiments;
R′ and R″ are each independently selected from a group consisting of hydrogen, an optionally substituted C₁₋₄ alkyl or an optionally substituted C₃₋₆ cycloalkyl; or
B₃ and R″ form a 6-membered heterocyclic group with the amido to which they are attached.

In one or more embodiments of the compound of Formula IIIa and Formula IIIb, R₁ is an optionally substituted C₁₋₃ alkyl or C₃₋₆ cycloalkyl. Preferably, R₁ is C₁₋₃ alkyl, halogenated C₁₋₃ alkyl or C₃₋₄ cycloalkyl.

In one or more embodiments of the compound of Formula IIIa and Formula IIIb, when R₂ is substituted, the substituents can be 1-5 groups selected from a group consisting of halogen and hydroxyl. Preferably, R₂ is hydrogen, halogen, an optionally substituted C₁₋₃ alkyl and an optionally substituted C₁₋₃ alkoxy; more preferably, R₂ is hydrogen, C₁₋₃ alkyl or halogen. In some preferred embodiments, only one of A₁, A₂ and A₃ is N, and the other two are independently CR₂, preferably, R₂ is independently H, C₁₋₃ alkyl or halogen. In more preferred embodiment, A₃ is CH, one of A₁ and A₂ is N and the other is CR₂, wherein R₂ is H, C₁₋₃ alkyl or halogen. In some embodiments, A₁ is N, and both of A₂ and A₃ are CH. In some embodiments, A₂ is N and both of A₁ and A₃ are CH. In some embodiments, all of A₁, A₂ and A₃ are CR₂, each R₂ is independently H, C₁₋₃ alkyl or halogen. Preferably, A₃ is CH, one of A₁ and A₂ is CR₂, wherein R₂ is H, C₁₋₃ alkyl or halogen; more preferably, both of A₂ and A₃ are CH, A₁ is CR₂, wherein R₂ is C₁₋₃ alkyl or halogen.

In one or more embodiments of the compound of Formula IIIa and Formula IIIb, B₁, B₂, B₃ and B₄ are independently selected from a group consisting of N and CR₇; R₇ is selected from a group consisting of hydrogen, halogen, C₁₋₄ alkyl, C₁₋₄alkoxy, halogenated C₁₋₄ alkyl, halogenated C₁₋₄ alkoxy and —NR′R″; R′ and R″ are each independently selected from a group consisting of hydrogen, C₁₋₄ alkyl, halogenated C₁₋₄ alkyl or C₃₋₆ cycloalkyl. Preferably, R₇ is hydrogen, C₁₋₃ alkyl or halogen. In some preferred embodiment, both of B₁ and B₂ are CH, B₃ is N, and B₄ is CR₇, wherein R₇ is hydrogen, C₁₋₃ alkyl or halogen.

In one or more embodiments of the compound of Formula IIIa and Formula IIIb, R′ and R″ are each independently selected from a group consisting of hydrogen, an optionally substituted C₁₋₃ alkyl, an optionally substituted C₃₋₆ cycloalkyl. Preferably, R′ is hydrogen; R″ is hydrogen, C₁₋₃ alkyl, halogenated C₁₋₃ alkyl or C₃₋₆ cycloalkyl.

One group of preferred compounds of Formula I in this disclosure is represented by compounds of Formula IV (including Formulae IVa and IVb):

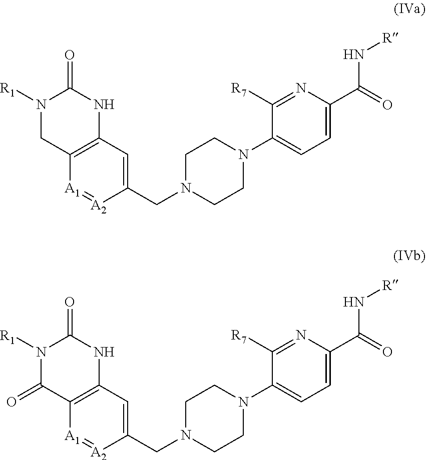

or stereoisomers, tautomers, N-oxides, hydrates, solvates, isotope-substituted derivatives, or pharmaceutically acceptable salts thereof, or mixtures thereof, or prodrugs thereof, wherein:
A₁, A₂, R₁, R₇ and R″ are as described in any of the foregoing embodiments.

In one or more embodiments of the compound of Formula IVa and Formula IVb, A1 and A₂ are each N and CR₂, wherein R₂ is preferably hydrogen, halogen, an optionally substituted C₁₋₃ alkyl or an optionally substituted C₁₋₃ alkoxy, more preferably R₂ is hydrogen, C₁₋₃ alkoxy or halogen. In some preferred embodiment, one of A₁ and A₂ is N and the other is CR₂, wherein R₂ is H, C₁₋₃ alkyl or halogen. In some embodiments, both of A₁ and A₂ are CR₂, wherein R₂ is H, C₁₋₃ alkyl, or halogen.

In one or more embodiments of the compound of Formula IVa and Formula IVb, R₁ is an optionally substituted C₁₋₃ alkyl or C₃₋₆ cycloalkyl. Preferably, R₁ is C₁₋₃ alkyl, halogenated C₁₋₃ alkyl or C₃₋₄ cycloalkyl.

In one or more embodiments of the compound of Formula IVa and Formula IVb, R″ is hydrogen, an optionally substituted C₁₋₃ alkyl, an optionally substituted C₃₋₆ cycloalkyl. Preferably, R″ is hydrogen, C₁₋₃ alkyl, C₃₋₄ cycloalkyl or halogenated C₁₋₃ alkyl.

In one or more embodiments of the compound of Formula IVa and Formula IVb, R₇ is selected from a group consisting of hydrogen, halogen, C₁₋₃ alkyl or halogenated

It should be understood that although R₁, A₁, A₂, A₃, L, Cy, R₅, B₁, B₂, B₃, B₄, D₁, D₂, D₃, D₄, R′, R″ and n are described separately above, the described features, especially the preferred features, can be arbitrarily combined to form the scope of different compounds of Formula I (including formulae II, III and IV) in this disclosure. For example, for the features described for R₂ of one formula, when the R₂ group also exists in other formulae, the feature can also be used to define the R₂ group of other formulae.

The preferred compounds of Formula I include, without limitation:
5-(4-((3-ethyl-2,4-dioxo-1,2,3,4-tetrahydropyrido[3,2-d]pyrimidin-7-yl)methyl)piperazin-1-yl)-N-methylpicolinamide (Example 1);
5-(4-((3-methyl-2,4-dioxo-1,2,3,4-tetrahydropyrido[3,2-d]pyrimidin-7-yl)methyl)piperazin-1-yl)-N-methylpicolinamide (Example 2);
5-(4-((3-isopropyl-2,4-dioxo-1,2,3,4-tetrahydropyrido[3,2-d]pyrimidin-7-yl)methyl)piperazin-1-yl)-N-methylpicolinamide (Example, 3);
5-(4-((3-ethyl-2,4-dioxo-1,2,3,4-tetrahydropyrido[3,2-d]pyrimidin-7-yl)methyl)piperazin-1-yl)-N,6-dimethylpicolinamide (Example 4);
5-(4-((3-ethyl-2,4-dioxo-1,2,3,4-tetrahydropyrido[4,3-d]pyrimidin-7-yl)methyl)piperazin-1-yl)-N-methylpicolinamide (Example 5);
5-(4-((3-ethyl-6-fluoro-2,4-dioxo-1,2,3,4-tetrahydropyrido[3,2-d]pyrimidin-7-yl)methyl)piperazin-1-yl)-N-methylpicolinamide (Example 6);
5-(4-((2,4-dioxo-3-propyl-1,2,3,4-tetrahydropyrido[3,2-d]pyrimidin-7-methyl)piperazin-1-yl)-N-methylpicolinamide (Example 7);
5-(4-((2,4-dioxo-3-(trifluoromethyl)-1,2,3,4-tetrahydropyrido[3,2-d]pyrimidin-7-yl)methyl)piperazin-1-yl)-N-methylpicolinamide (Example 8);
5-(4-((3-ethyl-2,4-dioxo-1,2,3,4-tetrahydropyrido[3,2-d]pyrimidin-7-yl)methyl)piperazin-1-yl)-6-fluoro-N-methylpicolinamide (Example 9);
5-(4-((3-ethyl-2,4-dioxo-1,2,3,4-tetrahydropyrido[3,2-d]pyrimidin-7-yl)methyl)piperazin-1-yl)-6-chloro-N-methylpicolinamide (Example 10);
5-(4-((3-ethyl-2,4-dioxo-1,2,3,4-tetrahydropyrido[3,2-d]pyrimidin-7-yl)methyl)piperazin-1-yl)-N-methyl-6-(trifluoromethyl)picolinamide (Example 11);
5-(4-((3-ethyl-2,4-dioxo-1,2,3,4-tetrahydroquinazolin-7-yl)methyl)piperazin-1-yl)-N-methylpicolinamide (Example 12);
5-(4-((3-ethyl-2,4-dioxo-1,2,3,4-tetrahydroquinazolin-7-yl)methyl)piperazin-1-yl)-6-fluoro-N-methylpicolinamide (Example 13);
5-(4-((3-ethyl-6-fluoro-2,4-dioxo-1,2,3,4-tetrahydroquinazolin-7-yl)methyl)piperazin-1-yl)-6-fluoro-N-methylpicolinamide (Example 14);
5-(4-((3-ethyl-2-oxo-1,2,3,4-tetrahydroquinazolin-7-yl)methyl)piperazin-1-yl)-N-methylpicolinamide (Example 15);
5-(4-((1-ethyl-2-oxo-2,3-dihydro-1H-benzo[d]imidazol-5-yl)methyl)piperazin-1-yl)-N-methylpicolinamide (Example 16);
5-(4-((3-methyl-2,4-dioxo-1,2,3,4-tetrahydroquinazolin-7-yl)methyl)piperazin-1-yl)-6-fluoro-N-methylpicolinamide (Example 17);
5-(4-((2,4-dioxo-3-(trifluoromethyl)-1,2,3,4-tetrahydroquinazolin-7-yl)methyl)piperazin-1-yl)-6-fluoro-N-methylpicolinamide (Example 18);
5-(4-((2,4-dioxo-3-propyl-1,2,3,4-tetrahydroquinazolin-7-yl)methyl)piperazin-1-yl)-6-fluoro-N-methylpicolinamide (Example 19);
5-(4-((3-(2-fluoroethyl)-2,4-dioxo-1,2,3,4-tetrahydroquinazolin-7-yl)methyl)piperazin-1-yl)-N,6-dimethylpicolinamide (Example 20);
5-(4-((3-ethyl-2,4-dioxo-1,2,3,4-tetrahydroquinazolin-7-yl)methyl)piperazin-1-yl)-6-chloro-N-methylpicolinamide (Example 21);
5-(4-((3-ethyl-2,4-dioxo-1,2,3,4-tetrahydroquinazolin-7-yl)methyl)piperazin-1-yl)-N,6-dimethylpicolinamide (Example 22);
5-(4-((3-ethyl-2,4-dioxo-1,2,3,4-tetrahydroquinazolin-7-yl)methyl)piperazin-1-yl)-N-methyl-6-(trifluoromethyl)picolinamide (Example 23);
5-(4-((3-ethyl-8-fluoro-2,4-dioxo-1,2,3,4-tetrahydroquinazolin-7-yl)methyl)piperazin-1-yl)-6-fluoro-N-methylpicolinamide (Example 24);
5-(4-((3-ethyl-5-fluoro-2,4-dioxo-1,2,3,4-tetrahydroquinazolin-7-yl)methyl)piperazin-1-yl)-6-fluoro-N-methylpicolinamide (Example 25);
5-(4-((3-isopropyl-2,4-dioxo-1,2,3,4-tetrahydroquinazolin-7-yl)methyl)piperazin-1-yl)-6-fluoro-N-methylpicolinamide (Example 26);
5-(4-((3-ethyl-2,4-dioxo-1,2,3,4-tetrahydroquinazolin-7-yl)methyl)piperazin-1-yl)-N-methyl)pyrimidine-2-carboxamide (Example 27);
6-(4-((3-ethyl-2,4-dioxo-1,2,3,4-tetrahydroquinazolin-7-yl)methyl)piperazin-1-yl)-N-methylnicotinamide (Example 28);
6-(4-((3-ethyl-2,4-dioxo-1,2,3,4-tetrahydroquinazolin-7-yl)methyl)piperazin-1-yl)-N-methylpyridazine-3-carboxamide (Example 29);
2-(4-((3-ethyl-2,4-dioxo-1,2,3,4-tetrahydroquinazolin-7-yl)methyl)piperazin-1-yl)- N-methylpyrimidine-5-carboxamide (Example 30);
5-(4-((3-ethyl-2,4-dioxo-1,2,3,4-tetrahydroquinazolin-7-yl)methyl)piperazin-1-yl)-6-ethyl-N-methylpicolinamide (Example 31);
5-(4-((3-ethyl-2,4-dioxo-1,2,3,4-tetrahydroquinazolin-7-yl)methyl)piperazin-1-yl)-6-fluoropicolinamide (Example 32);
5-(4-((3-ethyl-2,4-dioxo-1,2,3,4-tetrahydroquinazolin-7-yl)methyl)piperazin-1-yl)-6-fluoro-N-ethylpicolinamide (Example 33);
5-(4-((3-ethyl-2,4-dioxo-1,2,3,4-tetrahydroquinazolin-7-yl)methyl)piperazin-1-yl)-6-fluoro-N-isopropylpicolinamide (Example 34);
5-(4-((3-ethyl-2,4-dioxo-1,2,3,4-tetrahydroquinazolin-7-yl)methyl)piperazin-1-yl)-6-(difluoromethyl)-N-methylpicolinamide (Example 35);
3-ethyl-7-((4-(2-methyl-1-oxo-1,2,3,4-tetrahydroisoquinolin-6-yl)piperazin-1-yl)methyl)quinazoline-2,4(1H,3H)-dione (Example 36);
3-ethyl-7-((4-(1-oxo-1,2,3,4-tetrahydroisoquinolin-6-yl)piperazin-1-yl)methyl)quinazoline-2,4(1H,3H)-dione (Example 37);
3-ethyl-7-((4-(4-oxo-4H-pyrido[1,2-a]pyrimidin-8-yl)piperazin-1-yl)methyl)quinazoline-2,4(1H,3H)-dione (Example 38);
5-(4-((3-ethyl-2,4-dioxo-1,2,3,4-tetrahydroquinazolin-7-yl)methyl)piperazin-1-yl)-6-fluoro-N,N-dimethylpicolinamide (Example 39);
5-(3-(3-methyl-2,4-dioxo-1,2,3,4-tetrahydroquinazolin-7-yl)benzamido)N-methylpicolinamide (Example 40);
5-(3-(3-ethyl-2,4-dioxo-1,2,3,4-tetrahydroquinazolin-7-yl)benzamido)-N-methylpicolinamide (Example 41);
5-(3-(3-isopropyl-2,4 dioxo-1,2,3,4-tetrahydroquinazolin-7-yl)benzamido)-N-methylpicolinamide (Example 42);
5-(3-(3-ethyl-2,4-dioxo-1,2,3,4-tetrahydroquinazolin-7-yl)-5-fluorobenzamido)-N-methylpicolinamide (Example 43);
5-(3-(3-ethyl-2,4-dioxo-1,2,3,4-tetrahydroquinazolin-7-yl)benzamido)-6-fluoro-N-methylpicolinamide (Example, 44);
5-(3-(3-ethyl-2,4-dioxo-1,2,3,4-tetrahydropyrido[3,2-d]pyrimidin-7-yl)benzamido)-1-methylpicolinamide (Example 45);
6-(3-ethyl-2,4-dioxo-1,2,3,4-tetrahydroquinazolin-7-yl) -N-(6-(methylcarbamoyl)pyridin-3-yl)picolinamide (Example 46);
5-(3-(3-propyl-2,4-dioxo-1,2,3,4-tetrahydroquinazolin-7-yl)benzamido)-N-methylpicolinamide (Example 47);
5-(4-((3-ethyl-2,4-dioxo-1,2,3,4-tetrahydroquinazolin-7-yl)methyl)piperazin-1-yl)-4-fluoro-N-methylpicolinamide (Example 48);
5-(4-((3-ethyl-2,4-dioxo-1,2,3,4-tetrahydroquinazolin-7-yl)methyl)piperazin-1-yl)-3-fluoro-N-methylpicolinamide (Example 49);
5-(4-((3-ethyl-2,4-dioxo-1,2,3,4-tetrahydroquinazolin-7-yl)methyl)piperazin-1-yl)-N,4-dimethylpicolinamide (Example 50);
5-(4-((3-ethyl-2,4-dioxo-1,2,3,4-tetrahydroquinazolin-7-yl)methyl)piperazin-1-yl)-N,3-dimethylpicolinamide (Example 51);
5-(4-((3-ethyl-6-methyl-2,4-dioxo-1,2,3,4-tetrahydroquinazolin-7-yl)methyl)piperazin-1-yl)-N,6-dimethylpicolinamide (Example 52);
5-(4-((6-chloro-3-ethyl-2,4-dioxo-1,2,3,4-tetrahydroquinazolin-7-yl)methyl)piperazin-1-yl)-N,6-dimethylpicolinamide (Example 53);
3-ethyl-7-((4-(2-methyl-6-(5-methyl-1,3,4-oxadiazol-2-yl)pyridin-3-yl)piperazin-1-yl)methyl)quinazoline-2,4(1H,3H)-dione (Example 54);
5-(4-((3-ethyl-2,4-dioxo-1,2,3,4-tetrahydroquinazolin-7-yl)methyl)piperazin-1-yl)-N,6-dimethylpyridine-2-sulfonamide (Example 55);
5-(4-((3-ethyl-5-fluoro-2,4-dioxo-1,2,3,4-tetrahydroquinazolin-7-yl)methyl)piperazin-1-yl)-N,6-dimethylpicolinamide (Example 56);
5-(4-((3-ethyl-2,4-dioxo-1,2,3,4-tetrahydroquinazolin-7-yl)methyl)piperazin-1-yl)-N-methylpyrazine-2-carboxamide (Example 57);
5-(1-((3-ethyl-2,4-dioxo-1,2,3,4-tetrahydroquinazolin-7-yl)methyl)piperidin-4-yl)-N,6-dimethylpicolinamide (Example 58);
5-(4-((5-chloro-3-ethyl-2,4-dioxo-1,2,3,4-tetrahydroquinazolin-7-yl)methyl)piperazin-1-yl)-N,6-dimethylpicolinamide (Example 59);
5-(4-((3-ethyl-5-methyl-2,4-dioxo-1,2,3,4-tetrahydroquinazolin-7-yl)methyl)piperazin-1-yl)-N,6-dimethylpicolinamide (Example 60);
6-chloro-5-(4-((3-ethyl-6-fluoro-2,4-dioxo-1,2,3,4-tetrahydroquinazolin-7-yl)methyl)piperazin-1-yl)-N-methylpicolinamide (Example 61);
6-chloro-5-(4-((3-ethyl-5-fluoro-2,4-dioxo-1,2,3,4-tetrahydroquinazolin-7-yl)methyl)piperazin-1-yl)-N-methylpicolinamide (Example 62);
6-chloro-5-(4-((5-chloro-3-ethyl-2,4-dioxo-1,2,3,4-tetrahydroquinazolin-7- yl)methyl)piperazin-1-yl)-N-methylpicolinamide (Example 63);
6-chloro-5-(4-((3-ethyl-5-methyl-2,4-dioxo-1,2,3,4-tetrahydoquinazolin-7-yl)methyl)piperazin-1-yl)-N-methylpicolinamide (Example 64);
6-chloro-5-(4-((6-chloro-3-ethyl-2,4-dioxo-1,2,3,4-tetrahydroquinazolin-7-yl)methyl)piperazin-1-yl)-N-methylpicolinamide (Example 65);
6-chloro-5-(4-((3-ethyl-6-methyl-2,4-dioxo-1,2,3,4-tetrahydroquinazolin-7-yl)methyl)piperazin-1-yl)-N-methylpicolinamide (Example 66);
5-(4-((3-ethyl-6-fluoro-2,4-dioxo-1,2,3,4-tetrahydroquinazolin-7-yl)methyl)piperazin-1-yl)-N,6-dimethylpicolinamide (Example 67);
4-(4-((3-ethyl-2,4-dioxo-1,2,3,4-tetrahydroquinazolin-7-yl)methyl)piperazin-1-yl)-N-methylbenzamide (Example 68);
4-(4-((3-ethyl-2,4-dioxo-1,2,3,4-tetrahydroquinazolin-7-yl)methyl)piperazin-1-yl)-3-fluoro-N-methyibenzamide (Example 69);
3-chloro-4-(4-((3-ethyl-2,4-dioxo-1,2,3,4-tetrahydroquinazolin-7-yl)methyl)piperazin-1-yl)-N-methylbenzamide (Example 70);
4-(4-((3-ethyl-2,4-dioxo-1,2,3,4-tetrahydroquinazolin-7-yl)methyl)piperazin- -yl)-N,3-dimethylbenzamide (Example 71);
5-(4-((3-ethyl-2,4-dioxo-1,2,3,4-tetrahydroquinazolin-7-yl)methyl)piperazin-1-yl)-N,4,6-trimethylpicolinamide (Example 72);
4-chloro-5-(4-((3-ethyl-2,4-dioxo-1,2,3,4-tetrahydroquinazolin-7-yl)methyl)piperazin-1-yl)-N,6-dimethylpicolinamide (Example 73);
5-(4-((3-ethyl-2,4-dioxo-1,2,3,4-tetrahydropyrido[2,3-d]pyrimidin-7-yl)methyl)piperazin-1-yl)-N,6-dimethylpicolinamide (Example 74);
5-(4-((3-ethyl-2,4-dioxo-1,2,3,4-tetrahydroquinazolin-7-yl)methyl)piperazin-1-yl)-N,4-(dimethylpyrimidine-2-carboxamide (Example 75);
5-(4-((3-ethyl-2,4-dioxo-1,2,3,4-tetrahydroquinazolin-7-yl)methyl)piperazin-1-yl)-N,6-dimethylpyrazine-2-carboxamide (Example 76);
6-(4-((3-ethyl-2,4-dioxo-1,2,3,4-tetrahydroquinazolin-7-yl)methyl)piperazin-1-yl)-N,5-dimethylpyridazine-3-carboxamide (Example 77);
N-cyclopropyl-5-(4-((3-ethyl-2,4-dioxo-1,2,3,4-tetrahydroquinazolin-7-yl)methyl)piperazin-1-yl)-6-methylpicolinamide (Example 78);
6-(4-((3-ethyl-2,4-dioxo-1,2,3,4-tetrahydroquinazolin-7-yl)methyl)piperazin-1-yl)-N,5-dimethylnicotinamide (Example 79);
5-(4-((3-ethyl-2,4-dioxo-1,2,3,4-tetrahydroquinazolin-7-yl)methyl)piperazin-1-yl)-6-isopropyl-N-methylpicolinamide (Example 80);
5-(4-((3-ethyl-2,4-dioxo-1,2,3,4-tetrahydropyrido[4,3-d]pyrimidin-7-yl)methyl)piperazin-1-yl)-N,6-dimethylpicolinamide (Example 81);
N-ethyl-5-(4-((3ethyl-5-fluoro-2,4-dioxo-1,2,3,4-tetrahydroquinazolin-7-yl)methyl)piperazin-1-yl)-6-methylpicolinamide (Example 82);
6-chloro-N-ethyl-5-(4-((3-ethyl-2,4-dioxo-1,2,3,4-tetrahydroquinazolin-7-yl)methyl)piperazin-1-yl)picolinamide (Example 83);
N-ethyl-5-(4-((3-ethyl-2,4-dioxo-1,2,3,4-tetrahydroquinazolin-7-yl)methyl)piperazin-1-yl)-6-methylpicolinamide (Example 84);
N,6-dimethyl-5-(4-((3-methyl-2,4-dioxo-1,2,3,4-tetrahydropyrido[3,2-d]pyrimidin-7-yl)methyl)piperazin-1-yl)picolinamide (Example 85);
6-chloro-N-methyl-5-(4-((3-methyl-2,4-dioxo-1,2,3,4-tetrahydropyrido[3,2-d]pyrimidin-7-yl)methyl)piperazin-1-yl)picolinamide (Example 86);
5-(4-((2,4-dioxo-3-(2,2,2-trifluoroethyl)-1,2,3,4-tetrahydroquinazolin-7-yl)methyl)piperazin-1-yl)-N,6-dimethylpicolinamide (Example 87);
5-(4-((3-ethyl-2,4-dioxo-1,2,3,4-tetrahydroquinazolin-7-yl)methyl)piperazin-1-yl)-4-fluoro-N,6-dimethylpicolinamide (Example 88);
5-(4-((3-ethyl-2,4-dioxo-1,2,3,4-tetrahydroquinazolin-7-yl)methyl)piperazin-1-yl)-N,3,6-trimethylpicolinamide (Example 89);
5-(4-((3-ethyl-2,4-dioxo-1,2,3,4-tetrahydroquinazolin-7-yl)methyl)piperazin-1-yl)-3-fluoro-N,6-dimethylpicolinamide (Example, 90);
3-chloro-5-(4-((3-ethyl-2,4-dioxo-1,2,3,4-tetrahydroquinazolin-7-yl)methyl)piperazin-1-yl)-N,6-dimethylpicolinamide (Example 91);
6-chloro-5-(4-((3-ethyl-2,4-dioxo-1,2,3,4-tetrahydroquinazolin-7-yl)methyl)piperazin-1-yl)-N,3-dimethylpicolinamide (Example 92);
6-chloro-5-(4-((3-ethyl-2,4-dioxo-1,2,3,4-tetrahydroquinazolin-7-yl)methyl)piperazin-1-yl)-N,4-dimethylpicolinamide (Example 93);
6-bromo-5-(4-((3-ethyl-2,4-dioxo-1,2,3,4-tetrahydroquinazolin-7-yl)methyl)piperazin-1-yl)-N-methylpicolinamide (Example 94);
6-bromo-N-ethyl-5-(4-((3-ethyl-2,4-dioxo-1,2,3,4-tetrahydroquinazolin-7-yl)methyl)piperazin-1-yl)picolinamide (Example 95);
N-ethyl-5-(4-((3-ethyl-2,4-dioxo-1,2,3,4-tetrahydroquinazolin-7-yl)methyl)piperazin-1-yl)-6-(trifluoromethyl)picolinamide (Example 96);
5-(4-((3-ethyl-2,4-dioxo-1,2:3,4-tetrahydroquinazolin-7-yl)methyl)piperazin-1-yl)-1-methyl-N-(trifluoromethyl)picolinamide (Example 97);
6-chloro-N-methyl-5-(4-((3-methyl-2,4-dioxo-1,2,3,4-tetrahydroquinazolin-7-yl)methyl)piperazin-1-yl)picolinamide (Example 98);
N,6-dimethyl-5-(4-((3-methyl-2,4-dioxo-1,2,3,4-tetrahydroquinazolin-7-yl)methyl)piperazin-1-yl)picolinamide (Example 99);
6-chloro-N-ethyl-5-(4-((3-methyl-2,4-dioxo-1,2,3,4-tetrahydroquinazolin-7-yl)methyl)piperazin-1-yl)picolinamide (Example 100);
N-ethyl-6-methyl-5-(4-((3-methyl-2,4-dioxo-1,2,3,4-tetrahydroquinazolin-7-yl)methyl)piperazin-1-yl)picolinamide (Example 101);
6-chloro-N-ethyl-5-(4-((3-ethyl-2,4-dioxo-1,2,3,4-tetrahydropyrido[3,2-d]pyrimidin-7-yl)methyl)piperazin-1-yl)picolinamide (Example 102);
N-ethyl-5-(4-((3-ethyl-2,4-dioxo-1,2,3,4-tetrahydropyrido[3,2-d]pyrimidin-7-yl)methyl)piperazin-1-yl)-6-methylpicolinamide (Example 103);
6-chloro-N-ethyl-5-(4-((3-ethyl-5-fluoro-2,4-dioxo-1,2,3,4-tetrahydroquinazolin-7-yl)methyl)piperazin-1-yl)picolinamide (Example 104);
5-(4-((5-fluoro-3-methyl-2,4-dioxo-1,2,3,4-tetrahydroquinazolin-7-yl)methyl)piperazin-1-yl)-N,6-dimethylpicolinamide (Example 105);
6-chloro-5-(4-((5-fluoro-3-methyl-2,4-dioxo-1,2,3,4-tetrahydroquinazolin-7-yl)methyl)piperazin-1-yl)-N-methylpicolinamide (Example 106);
N-ethyl-5-(4-fluoro-3-methyl-2,4-dioxo-1,2,3,4-tetrahydroquinazolin-7-yl)methyl)piperazin-1-yl)-6-methylpicolinamide (Example 107);
6-chloro-N-ethyl-5-(4-((5-fluoro-3-methyl-2,4-dioxo-1,2,3,4-tetrahydroquinazolin-7-yl)methyl)piperazin-1-yl)picolinamide (Example 108);
5-(4-((3-ethyl-2,4-dioxo-1,2,3,4-tetrahydropyrido[3,2-d]pyrimidin-7-yl)methyl)piperazin-1-yl)-6-methoxy-N-methylpicolinamide (Example 109);
7-((4-(1H-indol-6-yl)piperazin-1-yl)methyl)-3-ethylquinazoline-2,4(1H,3H)-dione (Example 110);
7-((4-(1H-indazol-6-yl)piperazin-1-yl)methyl)-3-ethylquinazoline-2,4(1H,3H)-dione (Example 111);
7-((4-(1H-indazol-5-yl)piperazin-1-yl)methyl)-3-ethylquinazoline-2,4(1H,3H)-dione (Example 112);
7-((4-(1H-benzo[d]imidazol-6-yl)piperazin-1-yl)methyl)-3-ethylquinazoline-2,4(1H,3H)-dione (Example 113);
5-(4-((3-ethyl-2-oxo-1,2,3,4-tetrahydroquinazolin-7-yl)methyl)piperazin-1-yl)-6-fluoro-N-methylpicolinamide (Example 114);
6-chloro-5-(4-((3-ethyl-2-oxo-1,2,3,4-tetrahydroquinazolin-7-yl)methyl)piperazin-1-yl3-N-methylpicolinamide (Example 115);
5-(4-((3-ethyl-2-oxo-1,2,3,4-tetrahydroquinazolin-7-yl)methyl)piperazin-1-yl)-N,6-diethylpicolinamide (Example 116);
5-(4-((3-ethyl-2-oxo-1,2,3,4-tetrahydroquinazolin-7-yl)methyl)piperazin-1-yl)-6-methyl-N-ethylpicolinamide (Example 117);
5-(4-((3-ethyl-2-oxo-1,2,3,4-tetrahydroquinazolin-7 yl)methyl)piperazin-1-yl)-6-chloro-N-ethylpicolinamide (Example 118);
5-(4-((3-ethyl-5-fluoro-2-oxo-1,2,3,4-tetrahydroquinazolin-7-yl)methyl)piperazin-1-yl)-N,6-dimethylpicolinamide (Example 119);
6-chloro-5-(4-((3-ethyl-5-fluoro-2-oxo-1,2,3,4-tetrahydroquinazolin-7-yl)methyl)piperazin-1-yl)-N-methylpicolinamide (Example 120);
N-ethyl-5-(4-((3-ethyl-5-fluoro-2-oxo-1,2,3,4-tetrahydroquinazolin-7-yl)methyl)piperazin-1-yl)-6-methylpicolinamide (Example 121);
6-chloro-N-ethyl-5-(4-((3-ethyl-5-fluoro-2-oxo-1,2,3,4-tetrahydroquinazolin-7-yl)methyl)piperazin-1-yl)picolinamide (Example 122);
5-(4-((3-ethyl-5-methoxy-2,4-dioxo-1,2,3,4-tetrahydroquinazolin-7-yl)methyl)piperazin-1-yl)-N,6-dimethylpicolinamide (Example 123);
6-chloro-5-(4-((3-ethyl-5-methoxy-2,4-dioxo-1,2,3,4-4-tetrahydroquinazolin-7-yl)methyl)piperazin-1-yl)-N-methylpicolinamide (Example 124);
5-(4-((5-chloro-3-ethyl-2,4-dioxo-1,2,3,4-tetrahydroquinazolin-7-yl)methyl)piperazin-1-yl)-N-ethyl-6-methylpicolinamide (Example 125);
6-chloro-5-(4-((5-chloro-3-ethyl-2,4-dioxo-1,2,3,4-tetrahydroquinazolin-7-yl)methyl)piperazin-1-yl)-N-ethylpicolinamide (Example 126);
5-(4-((5-chloro-3-ethyl-2-oxo-1,2,3,4-tetrahydroquinazolin-7-yl)methyl)piperazin-1-yl)-N,6-dimethylpicolinamide (Example 127);
6-chloro-5-(4-((5-chloro-3-ethyl-2-oxo-1,2,3,4-tetrahydroquinazolin-7-yl)methyl)piperazin-1-yl)-N-methylpicolinamide (Example 128);
5-(4-((5-chloro-3-ethyl-2-oxo-1,2,3,4-tetrahydroquinazolin-7-yl)methyl)piperazin-1-yl)-N-ethyl-6-methylpicolinamide (Example 129);
6-chloro-5-(4-((5-chloro-3-ethyl-2-oxo-1,2,3,4-tetrahydroquinazolin-7-yl)methyl)piperazin-1-yl)-N-ethylpicolinamide (Example 130);
6-chloro-5-(4-((5-chloro-3-ethyl-2,4-dioxo-1,2,3,4-tetrahydroquinazolin-7-yl)methyl)piperazin-1-yl)-N-cyclopropylpicolinamide (Example 131);
6-chloro-N-cyclopropyl-5-(4-((3-ethyl-2-oxo-1,2,3,4-tetrahydroquinazolin-7-yl)methyl)piperazin-1-yl)picolinamide (Example 132);
N-ethyl-5-(4-((3-ethyl-2-oxo-1,2,3,4-tetrahydroquinazolin-7-yl)methyl)piperazin-1-yl)-6-fluoropicolinamide (Example 133);
5-(4-((3-ethyl-5-fluoro-2-oxo-1,2,3,4-tetrahydroquinazolin-7-yl)methyl)piperazin-1-yl)-6-fluoro-N-methylpicolinamide (Example 134);
N-cyclopropyl-5-(4-((3-ethyl-2,4-dioxo-1,2,3,4-tetrahydroquinazolin-7-yl)methyl)piperazin-1-yl)-6-fluoropicolinamide (Example 135);
5-(4-((5-chloro-3ethyl-2,4-dioxo-1,2,3,4-tetrahydroquinazolin-7-yl)methyl)piperazin-1-yl)-6-fluoro-N-methylpicolinamide (Example 136);
N-cyclopropyl-5-(4-((3-ethyl-2-oxo-1,2,3,4-tetrahydroquinazolin-7-yl)methyl)piperazin-1-yl)-6-methylpicolinamide (Example 137);
6-fluoro-5-(4-((8-fluoro-3-methyl-2,4-dioxo-1,2,3,4-tetrahydroquinazolin-7-yl)methyl)piperazin-1-yl)-N-methylpicolinamide (Example 138);
6-fluoro-5-(4-((8-fluoro-3-methyl-2-oxo-1,2,3,4-tetrahydroquinazolin-7-yl)methyl)piperazin-1-yl)-N-methylpicolinamide (Example 139);
5-(4-((3-cyclopropyl-2,4-dioxo-1,2,3,4-tetrahydroquinazolin-7-yl)methyl)piperazin-1-yl)-6-fluoro-N-methylpicolinamide (Example 140);
N-cyclopropyl-5-(4-((3-ethyl-5-fluoro-2,4-dioxo-1,2,3,4-tetrahydroquinazolin-7-yl)methyl)piperazin-1-yl)-6-fluoropicolinamide (Example 141);
5-(4-((3-ethyl-5-methyl-2,4-dioxo-1,2,3,4-tetrahydroquinazolin-7-yl)methyl)piperazin-1-yl)-6-fluoro-N-methylpicolinamide (Example 142);
5-(4-((3-ethyl-5-methoxy-2,4-dioxo-1,2,3,4-tetrahydroquinazolin-7-yl)methyl)piperazin-1-yl)-6-fluoro-N-methylpicolinamide (Example 143);
5-(4-((3-ethyl-2-oxo-1,2,3,4-tetrahydropyrido[3,2-d]pyrimidin-7-yl)methyl)piperazin-1-yl)-N-methylpicolinamide (Example 144);
4-(4-((3-ethyl-2-oxo-1,2,3,4-tetrahydroquinazolin-7-1)methyl)piperazin-1-yl)-N,3-dimethylbenzamide (Example 145);
3-chloro-4-(4-((3-ethyl-2-oxo-1,2,3,4-tetrahydroquinazolin-7-yl)methyl)piperazin-1-yl)-N-methylbenzamide (Example 146);
   or stereoisomers, tautomers, N-oxides, hydrates, isotope-substituted derivatives, solvates or pharmaceutically acceptable salts thereof, or mixtures thereof, or prodrugs thereof.

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁-C₄, alcohol according to methods known in the art); esters of hydroxy containing compounds (e.g., those obtained by condensation with a C₁-C₄ carboxylic acid, C₃-C₆ diacid or anhydride thereof, such as succinic anhydride and fumaric anhydride according to methods known in the art); imines of amino containing compounds (e.g., those obtained by condensation with a C₁-C₄ aldehyde or ketone according to methods known in the art); carbamate of amino containing compounds, such as those described by Leu, et al., (J. Med. Chem. 42:3623-3628 (1999)) and Greenwald, et al., (J. Med Chem. 42:3657-3667 (1999)); and acetals and ketals of alcohol-containing compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including formulae II, III and IV) can be prepared as illustrated by the exemplary reaction in Scheme 1. The reaction of methyl 3-amino-5-bromopicolinate and Boc anhydride under the catalysis of DMAP and DIEA produced methyl 3-(bis(tert-butoxycarbonyl)amino)-5-bromopicolinate. Suzuki reaction of methyl 3-(bis(tert-butoxycarbonyl)amino)-5-bromopicolinate and trimethylboroxine under the catalysis of Pd(dppf)Cl₂ produced methyl 3-(bis(tert-butoxycarbonyl)amino)-5-methylpicolinate. The bromination reaction of methyl 3-(bis(tert-butoxycarbonyl)amino)-5-methylpicolinate and NBS under the catalysis of BPO produced methyl 3-(bis(tert-butoxycarbonyl)amino)-5-(bromomethyl)picolinate. The reaction of methyl 3-(bis(tert-butoxycarbonyl)amino)-5-(bromomethyl)picolinate and N-methyl-5-(piperazin-1-yl)picolinamide under the catalysis of DIEA produced methyl 3-(bis(tert-butoxycarbonyl)amino)-5-((4-(6-(methylcarbamoyl)pyridin-3-yl)piperazin-1-yl)methyl)picolinate. The Boc deprotection reaction of methyl 3-(bis(tert-butoxycarbonyl)amino)-5-((4-(6-(methylcarbamoyl)pyridin-3-yl)piperazin-1-yl)methyl)picolinate under the catalysis of TFA produced methyl 3-amino-5-((4-(6-(methylcarbamoyl)pyridin-3-yl)piperazin-1-yl)methyl)picolinate. The ring closure reaction of methyl 3-amino-5-((4-(6-(methylcarbamoyl)pyridin-3-yl)piperazin-1-yl)methyl)picolinate and ethyl isocyanate produced the target compound 5-(4-((3-ethyl-2,4-dioxo-1,2,3,4-tetrahydropyrido[3,2-d]pyrimidin-7-yl)methyl)piperazin-1-yl)-N-methylpicolinamide.

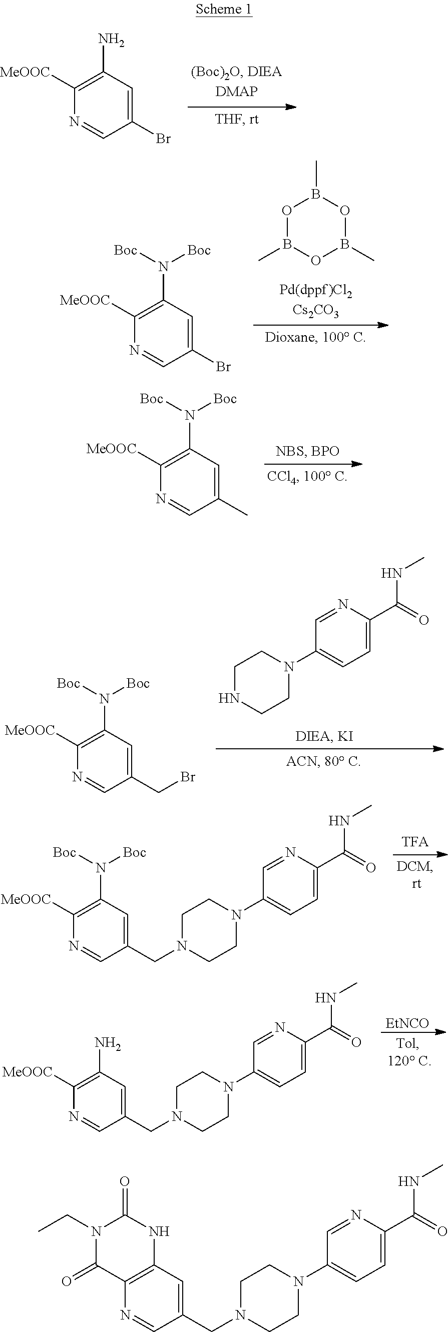

Other related compounds can be prepared using similar methods. For example, replacement of ethyl isocyanate with methyl isocyanate produced the target compound 5-(4-((3-methyl-2,4-dioxo-1,2,3,4-tetrahydropyrido[3,2-d]pyrimidin-7-yl)methyl)piperazin-yl)-N-methylpicolinamide. Replacement of ethyl isocyanate with isopropyl isocyanate produced the target compound 5-(4-((3-isopropyl-2,4-dioxo-1,2,3,4-tetrahydropyrido[3,2-d]pyrimidin-7-yl)methyl)piperazin-1-yl)-N-methylpicolinamide. Replacement of N-methyl-5-(piperazin-1-yl)picolinamide with 6-fluoro-N-methyl-5-(piperazin-1-yl)picolinamide produced the target compound 5-(4-((3-ethyl-2,4-dioxo-1,2,3,4,-tetrahydropyrido[3,2-d]pyrimidin-7-yl)methyl)piperazin-1-yl)-6-fluoro-N-methylpicolinamide.

The compounds of this disclosure can be prepared as illustrated by the exemplary reaction in Scheme 2. The heat reaction of dimethyl 2-aminoterephthalate and ethyl isocyanate in toluene in a sealed tube produced methyl 3-ethyl-2,4-dioxo-1,2,3,4-tetrahydroquinazoline-7-carboxylate. The reduction reaction of methyl 3-ethyl-2,4-dioxo-1,2,3,4-tetrahydroquinazoline-7-carboxylate and LiAlH₄ produced 3-ethyl-7-(hydroxymethyl)quinazoline-2,4(1H,3H)-dione. The Chlorination reaction of 3-ethyl-7-(hydroxymethyl)quinazoline-2,4(1H,3H)-dione and SOCl₂, produced 7-(chloromethyl)-3-ethylquinazoline-2,4(1H,3H)-dione. The substitution reaction of 7-(chloromethyl)-3-ethylquinazoline-2,4(1H,3H)-dione and N-methyl-5-(piperazin-1-yl)picolinamide produced the target compound 5-(4-((3-ethyl-2,4-dioxo-1,2,3,4-tetrahydroquinazolin-7-yl)methyl)piperazin-1-yl)-N-methylpicolinamide.

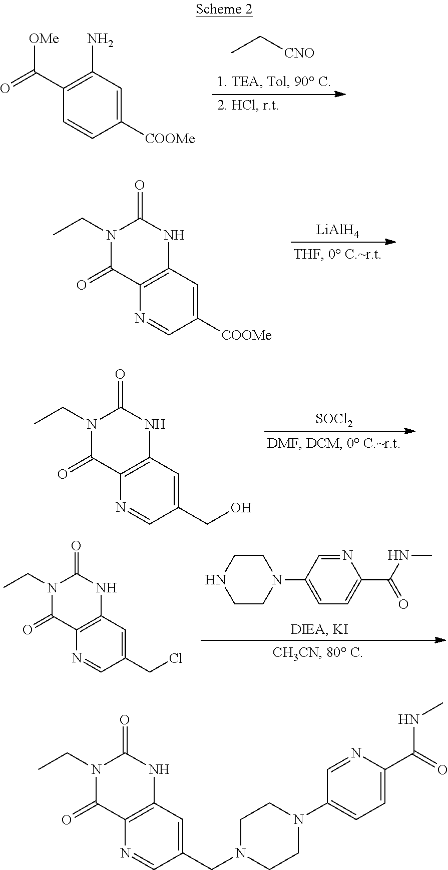

Other related compounds can be prepared using similar methods. For example, replacement of N-methyl-5-(piperazin-1-yl)picolinamide with 6-fluoro-N-methyl-5-(piperazin-1-yl)picolinamide produced the target compound 5-(4-((3-ethyl-2,4-dioxo-1,2,3,4-tetrahydroquinazolin-7-yl)methyl)piperazin-1-yl)-6-fluoro-N-methylpicolinamide. Replacement of dimethyl 2-aminoterephthalate with dimethyl 2-amino-5-fluoroterephthalate produced the target compound 5-(4-((3-ethyl-6-fluoro-2,4-dioxo-1,2,3,4-tetrahydroquinazolin-7-yl)methyl)piperazin-1-yl)-6-fluoro-N-methylpicolinamide. Replacement of ethyl isocyanate with propyl isocyanate produced the target compound 5-(4-((2,4-dioxo-3-propyl-1,2,3,4-tetrahydroquinazolin-7-yl)methyl)piperazin-1-yl)-6-fluoro-N-methylpicolinamide.

The compounds of this disclosure can be prepared as illustrated by the exemplary reaction in Scheme 3. 3-(4,4,5,5-tetramethyl-1,3,2-dioxaborolan-2-yl)benzoic acid reacted with (COCl)₂, then condensed with 5-amino-N-methylpicolinamide under the catalysis of TFA produced N-methyl-5-(3-(4,4,5,5-tetramethyl-1,3,2-dioxaborolan-2-yl)benzamido)picolinamide. Suzuki reaction of N-methyl-5-(3-(4,4,5,5-tetramethyl-1,3,2-dioxaborolan-2-yl)benzamido)picolinamide and 7-bromo-3-methylquinazoline-2,4(1H,3H)-dione under the catalysis of Pd(dppf)Cl₂ produced the target compound 5-(3-(3-methyl-2,4-dioxo-1,2,3,4-tetrahydroquinazolin-7-yl)benzamido)N-methylpicolinamide.

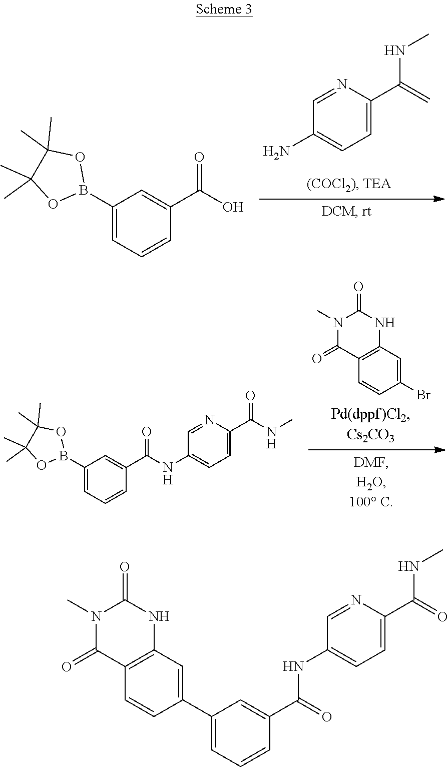

Other related compounds can be prepared using similar methods. For example, replacement of 7-bromo-3-methylquinazoline-2,4(1H,3H)-dione with 7-bromo-3-ethylquinazoline-2,4(1H,3H)-dione produced the target compound 5-(3-(3-ethyl-2,4-dioxo-1,2,3,4-tetrahydroquinazolin-7-yl)benzamido)-N-methylpicolinamide. Replacement of 7-bromo-3-methylquinazoline-2,4(1H,3H)- dione with 7-bromo-3-isopropylquinazoline-2,4(1H,3H)-dione produced the target compound 5-(3-(3-isopropyl-2,4-dioxo-1,2,3,4-tetrahydroquinazolin-7-yl)benzamido)-N-methylpicolinamide. Replacement of 7-bromo-3-methylquinazoline-2,4(1H,3H)-dione with 7-bromo-3-ethylpyrido[3,2-d]pyrimidine-2,4(1H,3H)-dione produced the target compound 5-(3-(3-ethyl-2,4-dioxo-1,2,3,4-tetrahydropyrido[3,2-d]pyrimidin-7-yl)benzamido)-N-methylpicolinamide. Replacement of 7-bromo-3-methylquinazoline-2,4(1H,3H)-dione with 7-bromo-3-propylquinazoline-2,4(1H,3H)-dione produced the target compound5-(3-(3-propyl-2,4-dioxo-1,2,3,4-tetrahydroquinazolin-7-yl)benzamido)-N-methylpicolinamide.

The compounds of this disclosure can be prepared as illustrated by the exemplary reaction in Scheme 4. The bromination reaction of methyl 4-methyl-3-nitrobenzoate and NBS under the catalysis of BPO produced methyl 4-(bromomethyl)-3-nitrobenzoate. The substitution reaction of methyl 4-(bromomethyl)-3-nitrobenzoate and ethylamine Under the catalysis of DIEA produced methyl 4-((ethylamino)methyl)-3-nitrobenzoate. The reduction reaction of methyl 4-((ethylamino)methyl)-3-nitrobenzoate and Fe/NH₄Cl produced methyl 3-amino-4-((ethylamino)methypbenzoate. The ring closure reaction of methyl 3-amino-4-((ethylamino)methyl)benzoate and CDI under heating produced methyl 3-ethyl-2-oxo-1,2,3,4-tetrahydroquinazoline-7-carboxylate. The reduction reaction of methyl 3-ethyl-2-oxo-1,2,3,4-tetrahydroquinazoline-7-carboxylate and LiAlH₄ produced 3-ethyl-7-(hydroxymethyl)-3,4-dihydroquinazolin-2(1H)-one. The Chlorination reaction of 3-ethyl-7-(hydroxymethyl)-3,4-dihydroquinazolin-2(1H)-one and SOCl₂ under the catalysis of DMF produced 7-(chloromethyl)-3-ethyl-3,4-dihydroquinazolin-2(1H)-one. The substitution reaction of 7-(chloromethyl)-3-ethyl-3,4-dihydroquinazolin-2(1H)-one and N-methyl-5-(piperazin-1-yl)picolinamide under the catalysis of DIEA produced the target compound 5-(4-((3-ethyl-2-oxo-1,2,3,4-tetrahydroquinazolin-7-yl)methyl)piperazin-1-yl)-N-methylpicolinanode.

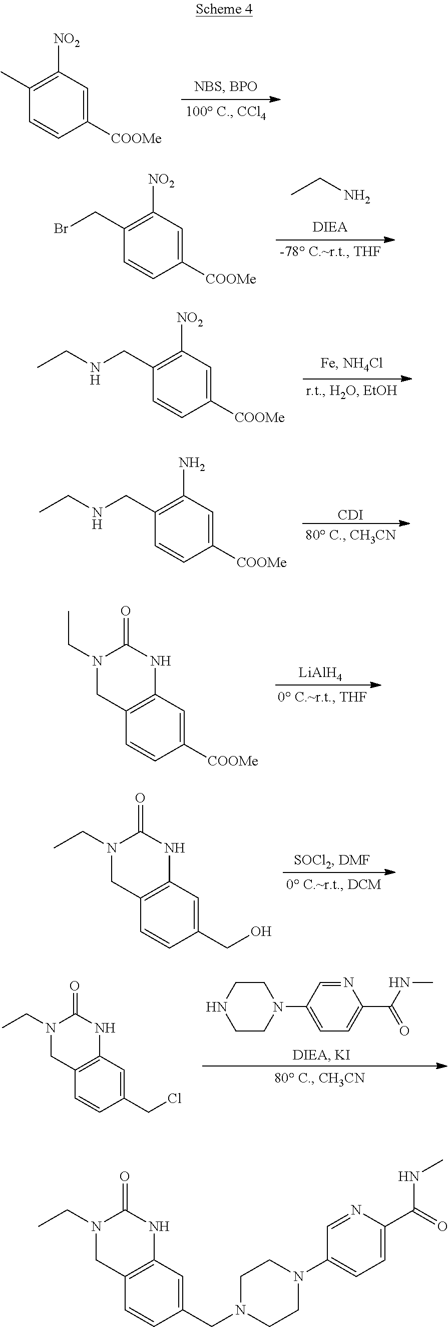

Other related compounds can be prepared using similar methods. For example, replacement of N-methyl-5-(piperazin-1-yl)picolinamide with 6-fluoro-N-methyl-5-(piperazin-1-yl)picolinamide produced the target compound 5-(4-((3-ethyl-2-oxo-1,2,3,4-tetrahydroquinazolin-7-yl)methyl)piperazin-1-yl)-6-fluoro-N-methylpicolinamide. Replacement of N-methyl-5-(piperazin-1-yl)picolinamide with 6-chloro-N-methyl-5-(piperazin-1-yl)picolinamide produced the target compound 6-chloro-5-(4-((3-ethyl-2-oxo-1,2,3,4-tetrahydroquinazolin-7-yl)methyl)piperazin-1-yl)-N-methylpicolinamide. Replacement of N-methyl-5-(piperazin-1-yl)picolinamide with N,6-dimethyl-5-(piperazin-1-yl)picolinamide produced the target compound 5-(4-((3-ethyl-2-oxo-1,2,3,4-tetrahydroquinazolin-7-yl)methyl)piperazin-1-yl)-N,6-dimethylpicolinamide.

An important aspect of the present disclosure is the discovery that compounds of Formula I (including Formulae II, III and IV) are PARP inhibitors, especially selective PARP1 inhibitors. Therefore, the compounds of Formula I (including Formulae II, III and IV) or stereoisomers, tautomers, N-oxides, hydrates, isotope-substituted derivatives, solvates or pharmaceutically acceptable salts thereof, or mixtures thereof, or prodrugs thereof can be used to treat a variety of diseases or conditions responsive to the inhibition of PARP activity (especially PARP1 activity), or used to prepare drug for treating diseases or conditions caused by responsive to the inhibition of PARP activity (especially PARP1 activity).

In the disclosure, the diseases or conditions responsive to the inhibition of PARP activity (especially PARP1 activity), includes cancer. Cancer can be a solid tumor or hematological tumor, including but is not limited to liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer, Wilms tumor, cervical cancer, testicular cancer, soft tissue sarcoma, primary macroglobulinemia, bladder cancer, chronic myeloid leukemia, primary brain cancer, malignant melanoma, small cell lung cancer, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer. Preferably, the cancer is responsive to the inhibition of PARP activity

Therefore, the present disclosure also includes methods for the treatment or prevention of diseases or conditions responsive to the inhibition of PARP activity (especially PARP1 activity), comprising administering to a subject (especially mammal, more specifically human) in need thereof an effective amount of the compound of Formula I (including Formulae II, III and IV) or stereoisomers, tautomers, N-oxides, hydrates, isotope-substituted derivatives, solvates or pharmaceutically acceptable salts thereof, or mixtures thereof, or prodrugs thereof, or a pharmaceutical composition comprising an effective amount of the compound of Formula I (including Formulae II, III and IV) or stereoisomers, tautomers, N-oxides, hydrates, isotope-substituted derivatives, solvates or pharmaceutically acceptable salts thereof, or mixtures thereof, or prodrugs thereof.

In practicing the therapeutic methods, effective amounts of pharmaceutical preparations are administered to an individual exhibiting the symptoms of one or more of these disorders. The pharmaceutic preparations comprise a therapeutically effective amount of the compound of Formula I (including Formulae II, III and IV), formulated for oral, intravenous, local or topical application, for the treatment of cancer and other diseases. The amount is effective to ameliorate or eliminate one or more symptoms of the disorders. An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I (including Formulae II, III and IV) as a PARP inhibitor, or stereoisomers, tautomers, N-oxides, hydrates, solvates, isotope-substituted derivatives, or pharmaceutically acceptable salts thereof, or mixtures thereof, or prodrugs thereof and a pharmaceutically acceptable carrier.

Another embodiment of the present disclosure is directed to a pharmaceutical composition effective to treat cancer comprising a compound of Formula I (including Formulae II, III and IV) as a PARP inhibitor, or stereoisomers, tautomers, N-oxides, hydrates, solvates, isotope-substituted derivatives, or pharmaceutically acceptable salts thereof and prodrugs thereof, in combination with at least one known anticancer agent or a pharmaceutically acceptable salt thereof. In particular, the compound herein can be combined with other anticancer drugs related to the mechanism of DNA damage and repair, including PARP inhibitors, such as olaparib, niraprib, rucaparib, talazoparib, pamiparib, fluzoparib and senaparib; HDAC inhibitors such as Volinota, Romididesin, Papiseta and Bailesta; and so on. And the compound herein can be combined with other anticancer drugs related to cell division detection sites, including Chk1/2 inhibitors, CDK4/6 inhibitors such as Paposinib, ATM/ATR inhibitors, Weel inhibitors, and so on. Other known anticancer agents which may be used for anticancer combination therapy include, but are not limited to alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxoruhicin, epirubicin, aclacinomycin, mitoxantrone, elliptinium and etoposide; RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelardbine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximdab, Blinatumomab, ipilimumab, avastin, herceptin and mabthera; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib, Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate, which functions as a kinase inhibitor, that comprises a compound described herein and is effective to inhibit tumor. The bioconjugate that inhibits tumor is consisted of the compound described herein and at least one known therapeutically useful antibody, such as trastuzumab or rituximab, or growth factor, such as EGF or FGF, or cytokine, such as IL-2 or 1L-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the PARP inhibitor of Formula I (including Formulae II, III and IV), or pharmaceutically acceptable salts thereof, or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the PARP inhibitor of Formula I (including Formulae II, III and IV), or pharmaceutically acceptable salts thereof, or prodrug thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, draggers, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane, N-methyl-glucamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner, e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, including, e.g., maize starch, wheat starch, rice starch, potato starch, gelatin, tragacanth, methylcellulose, hydroxypropyl methylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropyl methylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the manufacture of a medicament for the treatment of clinical symptoms in response to inhibition of the activity of PARP. The medicament may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform ii (Agilent 6110) quadrupole mass spectrometer fitted with an electrospray interface. ¹H NMR spectra was recorded at 400 MHz, on a Brücker Ascend 400 apparatus. Chemical shifts were recorded in parts per million (ppm) downfield from TMS (0.00 ppm), and J coupling constants were reported in hertz (Hz).

### Example 1

### 5-(4-((3-ethyl-2,4-dioxo-1,2,3,4-tetrahydropyrido[3,2-d]pyrimidin-7-yl)methyl)piperazin-1-yl)-N-methylpicolinamide

(a) Preparation of methyl 3-(bis(tert-butoxycarbonyl)amino)-5-bromopicolinate: To a solution of methyl 3-amino-5-bromopicolinate (3.0 g, 13.0 mmol) in THF (60 mL) was added (Boc)₂O (7.1 g, 32.6 mmol), DIEA (5.1 g, 39.1 mmol) and DMAP (318.2 mg, 2.6 mmol). The resulting mixture was stirred at room temperature overnight. After completion, the reaction mixture was concentrated under reduced pressure. The residue was diluted with water (30 mL) and extracted with DCM (30 mL×3). The combined organic phase was washed with brine, dried over Na₂SO₄ and concentrated under reduced pressure. The residue was purified by chromatography over silica gel (EtOAc/PE, 1 to 5%) to the title compound (3.5 g, white solid, yield: 62.5%). MS (ESI, m/z): 431.20 [M+1]⁺.

(b) Preparation of methyl 3-(bis(tert-butoxycarbonyl)amino)-5-methylpicolinate: To a solution of methyl 3-(bis(tert-butoxycarbonyl)amino)-5-bromopicolinate (3.0 g, 7.0 mmol) in dioxane (60 mL) was added 2,4,6-trimethyl-1,3,5,2,4,6-trioxatriborinane (6.0 mL, 21.0 mmol, 3.5 M in THF), Cs₂CO₃ (4.6 g, 14.0 mmol) and Pd(dppf)Cl₂ (768.6 mg, 1.05 mmol). The system was evacuated and backfilled with nitrogen three times. After stirred at 100° C. overnight, the mixture was filtered and the filtrate was concentrated under reduced pressure. The solvent was removed and the residue was purified by chromatography over silica gel (EtOAc/PE, 10 to 50%) to afford the title compound (2.3 g, white solid, yield: 88,2%), MS (ESI, m/z): 367.30 [M+1]⁺.

(c) Preparation of methyl 3-(bis(tert-butoxycarbonyl)amino)-5-(bromomethyl)picolinate: A solution of methyl 3-(bis(tert-butoxycarbonyl)amino)-5-methylpicolinate (50.0 mg, 0.14 mmol), NBS (24.3 mg, 0.14 mmol) and BPO (3.3 mg, 0.01 mmol) in CCl₄ (3 mL) was stirred at 100° C. overnight. LCMS showed starting material was remained. The mixture was added NBS (12.1 mg, 0.07 mmol) and stirred at 100° C. for another 6 h. After completion, the solvent was removed and the residue was purified by Prep-TLC (DCM/MeOH=10:1) to afford the title compound (30.0 mg, yellow solid, yield: 49.5%). MS (ESI, m/z): 445.30 [M+1]⁺.

(d) Preparation of methyl 3-(bis(tert-butoxycarbonyl)amino)-5-((4-(6-(methylcarbamoyl)pyridin-3-yl)piperazin-1-yl)methyl)picolinate: To a mixture of methyl 3-(bis(tert-butoxycarbonyl)amino)-5-methylpicolinate (30.0 mg, 0.07 mmol) in ACN (6 mL) was added N-methyl-5-(piperazin-1-yl)picolinamide (17.5 mg, 0.08 mmol), DIEA (43.2 mg, 0.34 mmol) and KI (1.1 mg, (0.01 mmol). The mixture was stirred at 80° C. overnight. After completion, the mixture was filtered and the filtrate was concentrated under reduced pressure. The residue was purified by Prep-TLC (DCM/MeOH=10:1) to give the title compound (40.0 mg crude) as a yellow solid, which was used for the next step directly without further purification. MS (ESI, m/z): 585.25 [M+1]⁺.

(e) Preparation of methyl 3-amino-5-((4-(6-(methylcarbamoyl)pyridin-3-yl)piperazin-1-yl)methyl)picolinate: To a solution of methyl 3-(bis(tert-butoxycarbonyl)amino)-5-((4-(6-(methylcarbamoyl)pyridin-3-yl)piperazin-1-yl)methyl)picolinate (40.0 mg 0.07 mmol) in DCM (5 mL) was added TFA (39.0 mg, 0.34 mmol). The resulting mixture was stirred at room temperature for 3 hours. After completion, the mixture was diluted with water (10 mL) and extracted with DCM (10 mL×3). The combined organic phase was washed with brine, dried over Na₂SO₄ and concentrated under reduced pressure to afford the title compound (30.0 mg crude) as a yellow solid, which was used for the next step directly without further purification. MS (ESI, m/z): 385.15 [M+1]⁺.

(f) Preparation of 5-(4-((3-ethyl-2,4-dioxo-1,2,3,4-tetrahydropyrido[3,2-d]pyrimidin-7-yl)methyl)piperazin-1-yl)-N-methylpicolinamide: To a solution of methyl 3-amino-5-((4-(6-(methylcarbamoyl)pyridin-3-yl)piperazin-1-yl)methyl)picolinate (30.0 nag, 0.08 mmol) in toluene (10 mL) was added Ethyl isocyanate (1 mL), TEA (1 mL). The resulting solution was stirred at 120° C. overnight. After completion, the residue was diluted with water (10 mL) and extracted with DCM (1.0 mL×3). The combined organic phase was washed with brine, dried over Na₂SO₄. and concentrated under reduced pressure. The residue was purified by Prep-TLC (DCM/ MeOH=10:1) and the impure product obtained was slurred with EA (2 mL) to afford the target compound (3.5 mg, white solid, yield: 12.3%, over 3 steps).

The following compounds of Examples 2-11 were prepared using a synthesis method similar to that described in Example 1.

| | | | LC-MS | |
|---|---|---|---|---|
| Example | Compound | MW | (ESI) | ¹H NMR (400 MHz) |
| 1 | 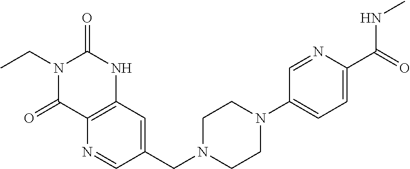 [SMILES: CCn1c(=O)[nH]c2cc(CN3CCN(c4ccc(C(=O)NC)nc4)CC3)cnc2c1=O] | 423.48 | 424.15 [M + H]⁺ | CD₃OD: δ 8.50 (d, J = 0,8 Hz, 1H), 8.28 (d, J = 2.2 Hz, 1H), 7.90 (d, J = 8.7 Hz, 1H), 7.67 (s, 1H), 7.37 (dd, J = 8.8, 2.6 Hz, 1H), 4.10 (q, J = 6.8 Hz, 2H), 3.74 (s, 2H), 3.44- 3.38 (m, 4H), 2.93 (s, 3H), 2.70-2.65 (m, 4H), 1.27 (t. J = 7.1 Hz, 3H). |
| 2 | 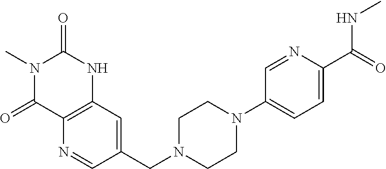 | 409.45 | 410.10 [M + H]⁺ | DMSO-d₆: δ 11.50 (s, 1H), 8.42 (s, 1H), 8.41- 8.31 (m, 1H), 8.24 (s, 1H), 7.80 (d, J = 8.9 Hz, 1H), 7.53 (s, 1H), 7.36 (d, J = 8.7 Hz, 1H), 3.65 (s, 2H), 3.32 (s, 3H), 3.26-3.21 (m, 4H), 2.75 (d, J = 4.6 Hz, 3H), 2.57- 2.50 (m, 4H). |
| 3 | 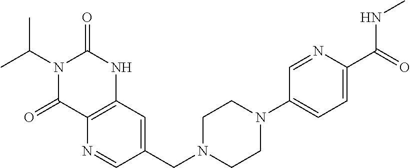 [SMILES: CNC(=O)c1ccc(N2CCN(Cc3cnc4c(=O)n(C(C)C)c(=O)[nH]c4c3)CC2)cn1] | 437.50 | 438.15 [M + H]⁺ | CDCl₃: δ 9.84 (s, 1H), 8.54 (s, 1H), 8.13 (d, J = 2.3 Hz, 1H), 8.05 (d, J = 8.8 Hz, 1H), 7.83 (d. J = 5.0 Hz, 1H), 7.60 (s, 1H), 7.16 (dd, J = 8.9, 2.8 Hz, 1H), 5.34 (dt, J = 14.3, 7.1 Hz, 1H), 3.69 (s, 2H), 3.32-3.25 (m, 4H), 3.03 (d, J = 5.0 Hz, 3H), 2.67-2.61 (m, 4H), 1.56 (d, J = 6.9 Hz, 6H). |
| 4 | 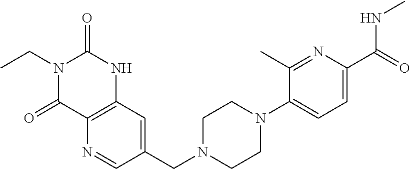 [SMILES: CCn1c(=O)[nH]c2cc(CN3CCN(c4ccc(C(=O)NC)nc4C)CC3)cnc2c1=O] | 437.50 | 438.10 [M + H]⁺ | DMSO-d₆: δ 11.47 (s, 1H), 8.51-8.37 (m, 2H), 7.79 (d, J = 8.2 Hz, 1H), 7.55 (s, 1H), 7.48 (d, J = 8.3 Hz, 1H), 4.04-3.84 (m, 2H), 3.69 (s, 2H), 3.02-2.88 (m, 4H), 2.79 (d, J = 4.8 Hz, 3H), 2.63-2.54 (m, 4H), 2.48 (s, 3H), 1.15 (t, J = 7.0 Hz, 3H). |
| 5 | 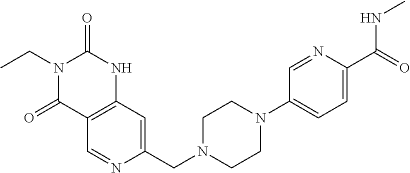 [SMILES: CCn1c(=O)[nH]c2cc(CN3CCN(c4ccc(C(=O)NC)nc4)CC3)ncc2c1=O] | 423.48 | 424.15 [M + H]⁺ | CDCl₃: δ 10.44 (s. 1H), 9.16 (s, 1H), 8.10- 8.01 (m, 2H), 7.99-7.81 (m, 1H), 7.39 (s, 1H), 7.09 (d. J = 8.8 Hz, 1H), 4.09 (q, J = 7.0 Hz, 2H), 3.77 (s, 2H), 3.30-3.17 (m, 4H), 3.05 (d, J = 5.0 Hz, 3H), 2.77-2.63 (m, 4H), 1.27 (t, J = 6.8 Hz, 3H). |
| 6 | 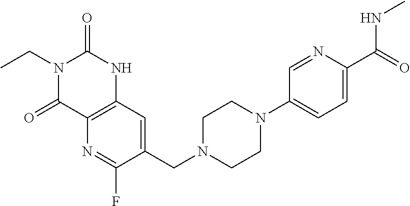 [SMILES: CCn1c(=O)[nH]c2cc(CN3CCN(c4ccc(C(=O)NC)nc4)CC3)c(F)nc2c1=O] | 441.47 | / | / |
| 7 | 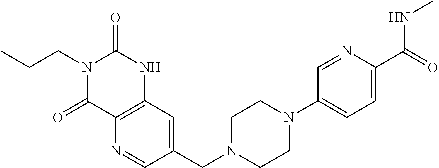 [SMILES: CCCn1c(=O)[nH]c2cc(CN3CCN(c4ccc(C(=O)NC)nc4)CC3)cnc2c1=O] | 437.50 | 438.15 [M + H]⁺ | CDCl₃: δ 10.15 (s, 1H), 8.57 (s, 1H), 8.12(d, J = 2.6 Hz, 1H), 8.05 (d, J = 8.8 Hz, 1H), 7.86 (d. J = 5.0 Hz, 1H), 7.67 (s, 1H), 7.16 (dd, J = 8.8, 2.8 Hz, 1H), 4.17-3.93 (m, 2H), 3.70 (s, 2H), 3.28 (d, J = 4.8 Hz, 4H), 3.04 (d, J = 5.1 Hz, 3H), 2.73-2.53 (m, 4H). 1.75 (q, J = 7.5 Hz, 2H), 0.98 (t, J = 7.4 Hz, 3H). |
| 8 | 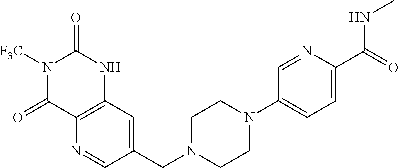 [SMILES: CNC(=O)c1ccc(N2CCN(Cc3cnc4c(=O)n(C(F)(F)F)c(=O)[nH]c4c3)CC2)cn1] | 463.42 | / | / |
| 9 | 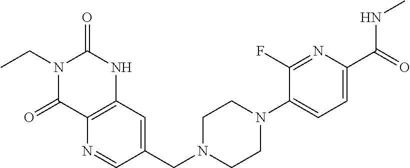 [SMILES: CCn1c(=O)[nH]c2cc(CN3CCN(c4ccc(C(=O)NC)nc4F)CC3)cnc2c1=O] | 441.47 | 442.10 [M + H]⁺ | DMSO-d₆: δ 8.44 (d, J = 1.7 Hz, 1H), 8.39 (q, J = 4.9 Hz, 1H), 7.84 (d, J = 8.3 Hz, 1H), 7.62-7.52 (m, 2H), 3.93 (9, J = 7.6 Hz, 2H), 3.67 (s, 2H), 3.23-3.12 (m, 4H), 2.76 (d, J = 4.8 Hz, 3H), 2.60-2.55 (m, 4H), 1.15 (t, J = 7.0 Hz, 3H). |
| 10 | 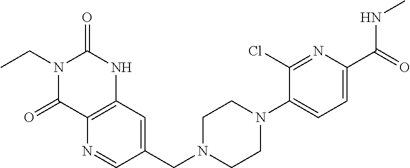 [SMILES: CCn1c(=O)[nH]c2cc(CN3CCN(c4ccc(C(=O)NC)nc4Cl)CC3)cnc2c1=O] | 457.92 | 458.15 [M + H]⁺ | CDCl₃: δ 9.38 (s, 1H), 8.60 (s, 1H), 8.09 (d, J = 8.1 Hz, 1H), 7.71 (d. J = 4.4 Hz, 1H), 7.56 (s, 1H), 7.39 (d, J = 8.1 Hz, 1H), 4.19 (q, J = 7.0 Hz, 2H), 3.72 (s, 2H), .322-3.15 (m, 4H), 3.03 (d, J = 5.0 Hz, 3H), 2.74-2.66 m, 4H), 1.32 (d, J = 7.0 Hz, 3H). |
| 11 | 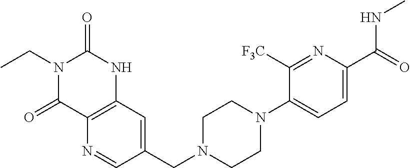 [SMILES: CCn1c(=O)[nH]c2cc(CN3CCN(c4ccc(C(=O)NC)nc4C(F)(F)F)CC3)cnc2c1=O] | 491.48 | / | / |

### Example 12

### 5-(4-((3-ethyl-2,4-dioxo-1,2,3,4-tetrahydroquinazolin-7-yl)methyl)piperazin-1-yl)-N -methylpicolinamide

(a) Preparation of methyl 3-ethyl-2,4-dioxo-1,2,3,4-tetrahydroquinazoline-7-carboxylate: A mixture of dimethyl 2-aminoterephthalate (200.0 mg, 1.0 mmol), ethyl isocyanate (228.0 mg, 3.2 mmol) and triethylamine (180.0 mg, 1.6 mmol) in toluene (3.0 mL) was heated at 90° C. overnight in a sealed tube. The mixture was concentrated. Methanol (5 mL) and concentrated hydrochloric acid (3 mL) was added and the mixture was stirred at room temperature overnight. After concentration, the residue was washed with water (20 mL) and methanol (20 mL), dried to give the title compound (crude, 480.0 mg, white solid). MS (ESL, m/z): 249.10 [M+1]⁺, 247.00 [M−1]⁻.

(b) Preparation of 3-ethyl-7-(hydroxymethyl)quinazoline-2,4(1H,3H)-dione: A suspension of lithium aluminum hydride (62.0 mg, 1.6 mmol) in THF (5 mL) was added methyl 3-ethyl-2,4-diaxo-1,2,3,4-tetrahydroquinazoline-7-carboxylate (200.0 mg, 0.8 mmol) under N₂ at 0° C. The mixture was warmed to room temperature and stirred for 2 hours, then quenched with 1 M HCl (2 mL). The mixture was concentrated and diluted with water (10 mL) to give a yellow suspension. The solid was collected by filtration, washed with water (10 mL), diethyl ether (10 mL) and dried to give the title compound (80.0 mg, yellow solid, yield: 91%, over 2 steps). MS (ESI, m/z): 221.20 [M+1]⁺, 219.15 [M−1]⁻.

(c) Preparation of 7-(chloromethyl)-3-ethylquinazoline-2,4(1H,3H)-dione: To a suspension of 3-ethyl-7-(hydroxymethyl) quinazoline-2,4(1H,3H)-dione (80.0 ) mg, 0.2 mmol) in DCM (3 mL) was added DMF (2.4 mg, 0.03 mmol) and thionyl chloride (231.0 mg, 1.9 mmol) dropwise and at 0° C. The resulting mixture was stirred at room temperature for 2 hours. After completion, the mixture was concentrated to give the title compound (crude, 70.0 mg, grey solid). MS (ESI, m/z): 239.35 [M+1]⁺, 237.1.0 [M−1]⁻.

(d) Preparation of 5-(4-((3-ethyl-2,4-dioxo-1,2,3,4-tetrahydroquinazolin-7-yl)methyl)piperazin-1-yl)-N-methylpicolinamide: To a solution of 7-(chloromethyl)-3-ethylquinazoline-2,4(1H,3H)-dione (70.0 mg, crude from above), KI (11.0 mg, 0.1mmol) and N-methyl-5-(piperazin-1-yl)picolinamide hydrochloride (71.0 mg, 0.3 mmol) in CH₃CN (4 mL) was added DIEA (209.0 mg, 1.6 mmol) at room temperature. The resulting solution was stirred at 80° C. for 2 hours. After completion, the solvent was removed under vacuum. The crude was diluted with water (10 mL) and the suspension was filtered. The filter cake was washed with methanol (10 mL) and ethyl acetate (10 mL), dried to give the target compound (35 mg, grey powder, yield: 23%, over 2 steps).

The following compounds of Examples 13-14 were prepared using a synthesis method similar to that described in Example 12.

| | | | LC-MS | |
|---|---|---|---|---|
| Example | Compound | MW | (ESI) | ¹H NMR (400 MHz) |
| 12 | 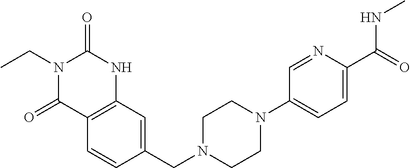 [SMILES: CCn1c(=O)[nH]c2cc(CN3CCN(c4ccc(C(=O)NC)nc4)CC3)ccc2c1=O] | 422.49 | 423.40 [M + H]⁺ | DMSO-d₆: δ 11.35 (s, 1H), 8.38 (d, J = 3.6 Hz, 1H), 8.24 (s, 1H), 7.87 (d, J = 8.2 Hz, 1H), 7.80 (d, J = 8.8 Hz, 1H), 7.36 (d, J = 8.6 Hz, 1H), 7.19-7.12 (m, 2H), 3.90 (d, J = 6.8 Hz, 2H), 3.57 (s, 2H), 3.29 (s, 3H), 2.84-2.67 (m, 4H), 2.55-2.50 (m, 4H), 1.11 (t, J = 6.8 Hz, 3H). |
| 13 | 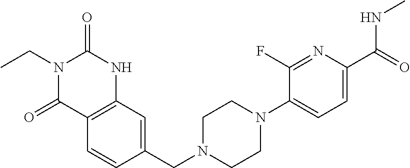 [SMILES: CCn1c(=O)[nH]c2cc(CN3CCN(c4ccc(C(=O)NC)nc4F)CC3)ccc2c1=O] | 440.48 | 441.30 [M + H]⁺ | CD₃OD: δ 7.98 (d, J = 7.9 Hz, 1H), 7.87 (d, J = 7.4 Hz, 1H), 7.49 (t, J = 9.4 Hz, 1H), 7.24 (d, J = 8.4 Hz, 1H), 7.20 (s, 1H), 4.05 (d, J = 7.0 Hz, 2H), 3.65 (s, 2H), 3.25 (m, 4H), 2.89 (s, 3H), 2.65 (m, 4H), 1.23 (t, J = 6,9 Hz, 3H). |
| 14 | 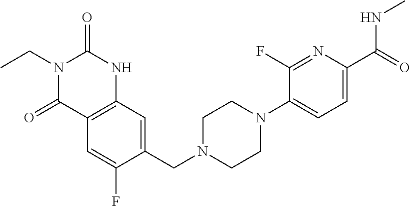 [SMILES: CCn1c(=O)[nH]c2cc(CN3CCN(c4ccc(C(=O)NC)nc4F)CC3)c(F)cc2c1=O] | 458.47 | 459.30 [M + H]⁺ | CDCl₃: δ 9.32 (s, 1H), 7.96 (d, J = 7.7 Hz, 1H), 7.74 (d, J = 9.2 Hz, 1H), 7.48 (s, 1H), 7.25 (d, J = 10.0 Hz, 1H), 4.08 (d, J = 7.0 Hz, 2H), 3.67 (s, 2H), 3.25-3.20 (m, 4H), 2.97 (d, J = 5.0 Hz, 3H), 22.73-2.68 (m, 4H), 1.23 (dd, J = 13.9, 7.1 Hz, 3H). |

### Example 15

### 5-(4-((3-ethyl-2-oxo-2,3,4-tetrahydroquinazolin-7-yl)methyl)piperazin-1-yl)-N-methylpicolinamide

(a) Preparation of methyl 4-(bromomethyl)-3-nitrobenzoate: A solution of methyl 4-methyl-3-nitrobenzoate (6.9 g, 35.4 mmol), NBS (6.3 g, 35.4 mmol) and BPO (858.0 mg, 3.5 mmol) in CCl₄ (140 mL) was stirred at 100° C. for 15 hours. After completion, the solvent was removed and the residue was purified by chromatography over silica gel (EtOAc/PE, 2 to 5%) to afford the title compound (4.2 g, yellow solid, yield: 43.3%).

(b) Preparation of methyl 4-((ethylamino)methyl)-3-nitrobenzoate: To a mixture of ethylamine (6.9 mL, 13.8 mmol, 2M in THF), DIEA (1.4 g, 10.4 mmol) in THF (20 mL) was added methyl 4-(bromomethyl)-3-nitrobenzoate (1.9 g in 10 mL THF, 6.9 mmol) at −78° C. The mixture was stirred at room temperature overnight. After completion, the solvent was removed and the residue was purified by chromatography over silica gel (EtOAc/PE, 10 to 20%) to afford the title compound (1.2 g, yellow solid, yield: 72.7%). MS (ESI, m/z): 239.20 [M+1]⁺.

(c) Preparation of methyl 3-amino-4-((ethylamino)methyl)benzoate: To a solution of methyl 4-((ethylamino)methyl)-3-nitrobenzoate (1.0 g, 4.2 mmol) in EtOH (30 mL) was added Fe (941.0 mg, 16.8 mmol) and NH₄Cl (2.2 g in 15 ML H₂O, 42.0 mmol). The resulting mixture was stirred at room temperature for 3 hours. After completion, the reaction mixture was filtered and the filter cake was washed with EtOH (50 mL), the mixture was concentrated under reduced pressure to give the residue which was diluted with water (50 mL) and extracted with DCM (50 mL×3). The combined organic phase was removed and the residue was purified by chromatography over silica gel (EtOAc/PE, 10 to 50%) to afford the title compound (400.0 mg, yellow solid, yield: 46.0%). MS (ESI, m/z): 209.05 [M+1]⁺.

(d) Preparation of methyl 3-ethyl-2-oxo-1,2,3,4-tetrahydroquinazoline-7-carboxylate: To a suspension of methyl 3-amino-4-((ethylamino)methyl)benzoate (200.0 mg, 1.0 mmol) in CH₃CN (5 mL) was added CDI (623.1 mg, 3.9 mmol) under N₂. The resulting mixture was stirred at 80° C. overnight. After completion, the mixture was concentrated. The residue was purified by Prep-TLC (P/E=1/1) to give the title compound (40.0 mg, yellow solid, yield: 17.8%). MS (ESI, m/z): 235.00 [M+1]⁺.

(e) Preparation of 3-ethyl-7-(hydroxymethyl)-3,4-dihydroquinazolin-2(1H)-one: A suspension of lithium aluminum hydride (26.0 mg, 0.7 mmol) in THF (5 mL) was added methyl 3-ethyl-2-oxo- 1,2,3,4-tetrahydroquinazoline-7-carboxylate (40.0 mg, 0.2 mmol) under N₂ at 0° C. The mixture was warmed to room temperature and stirred for 2 hours, then quenched with 1 M HCl (1 mL). The mixture was concentrated and diluted with water (10 mL) to give a yellow suspension. The solid was collected by filtration, washed with water (10 mL), diethyl ether (10 mL) and the filtrate was removed to give the title compound (35.0 mg, yellow solid, yield: 99.0%). MS (ESI, m/z): 207.10 [M+1]⁺.

(f) Preparation of 7-(chloromethyl)-3-ethyl-3,4-dihydroquinazolin-2(1H)-one: To a suspension of 3-ethyl-7-(hydroxymethyl)-3,4-dihydroquinazolin-2(1H)-one (35.0 mg, 0.2 mmol) in DCM (3 mL) was added DMF (1 drop) and thionyl chloride (80.9 mg, 0.7 mmol) dropwise at 0° C. The resulting mixture was stirred at room temperature for 2 hours. After completion, the mixture was concentrated to give the title compound (crude, 30.0 mg, grey solid, yield: 61.2%), which was used next step directly. MS (ESI, m/z): 225.05 [M+1]⁺.

(g) Preparation of 5-(4-((3-ethyl-2-oxo-1,2,3,4-tetrahydroquinazolin-7-yl)methyl)piperazin-1-yl)-N-methylpicolinamide: To a solution of 7-(chloromethyl)-3-ethyl-3,4-dihydroquinazolin-2(1H)-one (30.(l mg, 0.13 mmol), KI (4.4 mg, 0.03 mmol) and N-methyl-5-(piperazin-1-yl)picolinamide hydrochloride (37.6 mg, 0.16 mmol) in CH₃CN (4 mL) was added DIEA (86.4 mg, 0.67 mmol, 5.0 eq) at room temperature. The resulting solution was stirred at 80° C. for 2 hours. After completion, the solvent was removed under vacuum. The crude was diluted with water (10 mL) and the suspension was filtered. The filter cake was washed with methanol (10 mL) and ethyl acetate (10 mL), dried to give the target compound (22.8 mg, yellow solid, yield: 41.7%). MS (ESI, m/z): 409.10 [M+1]⁺. CDCl₃: δ 8.96 (s, 1H), 8.45 (d, J=8.0 Hz, 1H), 8.36 (d, J=7.9 Hz, 1H), 7.86 (d, J=5.4 Hz, 1H), 7.69-7.63 (m, 1H), 7.58 (d, J=8.1 Hz, 1H), 7.42 (s, 1H), 4.42-4.36 (m, 2H), 3.99 (s, 2H). 3.62-3.57 (m, 4H), 3.35 (d, J=4.6 Hz, 3H), 3.05-2.99 (m, 4H), 2.09 (dd, J=14.5, 7.3 Hz, 2H), 1.34 (t, J=7.2 Hz, 3H).

The following compounds of Examples 16-39 were prepared using a synthesis method. similar to that described in Example 12.

| Ex- | | | LC-MS | |
|---|---|---|---|---|
| ample | Compound | MW | (ESI) | ¹H NMR (400 MHz) |
| 16 | 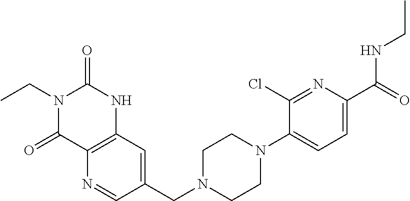 | 394.48 | 395.40 [M + H]⁺ | DMSO-d₆: δ 10.77 (s, 1H), 8.43-8.34 (m, 1H), 8.25 (d, J = 2.8 Hz, 1H), 7.81 (d, J = 8.8 Hz, 1H), 7.37 (dd, J = 8.8, 2.9 Hz, 1H), 7.07 (d, J = 8.3 Hz, 1H), 6.96 (d, J = 6.6 Hz, 2H), 3.80 (q, J = 7.5, 7.1 Hz, 2H), 3.51 (s, 2H), 3.28-3.26 (m, 8H), 2.77 (d, J = 4.8 Hz, 3H), 1.19 (t, J = 7.1 Hz, 3H). |
| 17 | 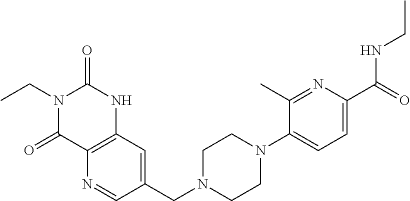 | 426.45 | 427.05 [M + H]⁺ | CDCl₃: δ 7.86 (d, J = 8.1 Hz, 1H), 7.74 (d, J = 8.1 Hz, 1H), 7.26 (s, 1H), 7.22-7.13 (m, 1H), 7.05 (d, J = 8.0 Hz, 1H), 6.97 (s, 1H), 3.47 (s, 2H), 3.17 (s, 3zH), 3.07 (t, J = 4.7 Hz, 4H), 2.79 (s, 3H), 2.49 (t, J = 4.8 Hz, 4H). |
| 18 | 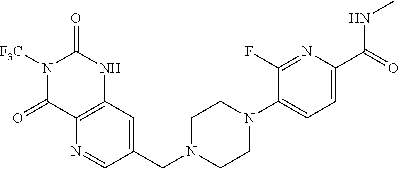 [SMILES: CNC(=O)c1ccc(N2CCN(Cc3cnc4c(=O)n(C(F)(F)F)c(=O)[nH]c4c3)CC2)c(F)n1] | 480.42 | / | / |
| 19 | 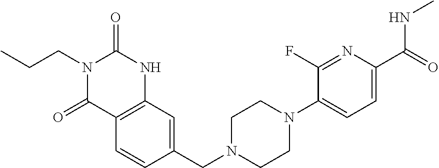 [SMILES: CCCn1c(=O)[nH]c2cc(CN3CCN(c4ccc(C(=O)NC)nc4F)CC3)ccc2c1=O] | 454.51 | 455.25 [M + H]⁺ | CDCl₃: δ 8.60 (s, 1H), 8.09 (d, J = 8.0 Hz, 1H), 8.00 (d, J = 7.9 Hz, 1H), 7.51 (m, 1H), 7.31 (d, J = 8.6 Hz, 1H), 7.22 (d. J = 8.1 Hz, 1H), 7.06 (s, 1H), 4.06-4.00 (m, 2H), 3.64 (s, 2H), 3.27- 3.22 (m, 4H), 3.00 (d, J = 4.6 Hz, 3H), 2.68- 2.62 (m, 4H), 1.78-1.68 (m. 2H), 0.98 (t, J = 7.2 Hz, 3H). |
| 20 | 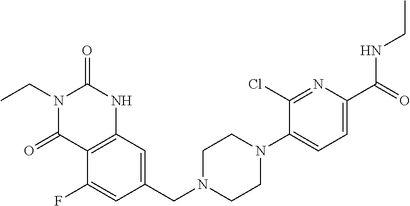 | 454.21 | 455.15 [M + H]⁺ | CD₃OD: δ 8.01 (d, J = 8.0 Hz, 1H), 7.86 (d, J = 8.3 Hz, 1H), 7.48 (d, J = 8.3 Hz, 1H), 7.28 (d, J = 8.7 Hz, 1H), 7.24 (s, 1H), 4.71 (t, J = 5.3 Hz, 1H), 4.59 (t, J = 5.3 Hz, 1H), 4.39 (t, J = 5.2 Hz, 1H), 4.33 (t, I = 5.2 Hz, 1H), 3.69 (s, 2H), 3.10-2.95 (m, 4H), 2.94 (s, 3H), 2.76-2.63 (m, 4H), 2.54 (s, 3H). |
| 21 | 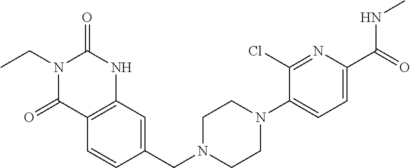 [SMILES: CCn1c(=O)[nH]c2cc(CN3CCN(c4ccc(C(=O)NC)nc4Cl)CC3)ccc2c1=O] | 456.93 | 457.10 [M + H]⁺ | CDCl₃: δ 8.44 (s, 1H), 8.09 (dd, J = 7.9, 6.4 Hz, 2H), 7.67 (d, J = 4.6 Hz, 1H), 7.39 (d, J = 8.1 Hz, 1H), 7.23 (d. J = 8.3 Hz, 1H), 7.06 (s, 1H), 4.13 (q, J = 7.0 Hz, 2H), 3.65 (s, 2H), 3.23-3.15 (m, 4H), 3.01 (d, J = 5.1 Hz, 3H), 2.73-2.65 (m, 4H), 1.30 (t, J = 7.0 Hz, 3H). |
| 22 | 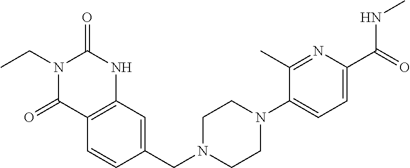 [SMILES: CCn1c(=O)[nH]c2cc(CN3CCN(c4ccc(C(=O)NC)nc4C)CC3)ccc2c1=O] | 436.52 | 437.15 [M + H]⁺ | CDCl₃:δ 8.37 (s, 1H), 8.09 (d, J = 7.9 Hz, 1H), 7.99 (d, J = 8.5 Hz, 1H), 7.95 (dd, J = 9.1, 5.3 Hz, 1H), 7.34 (d. J = 8.3 Hz, 1H), 7.22 (d, J = 8.9 Hz, 1H), 7.08 (s, 1H), 4.12 (q, J = 7.5 Hz, 2H), 3.65 (s, 2H), 3.03-3.00 (m, 4H), 3.00- 2.98 (m, 3H), 2.71-2.61 (m, 4H), 2.51 (s, 3H), 1.29 (t, J = 7.1 Hz, 3H). |
| 23 | 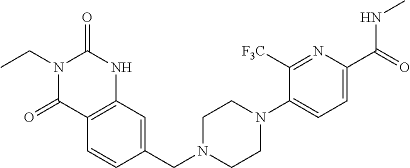 [SMILES: CCn1c(=O)[nH]c2cc(CN3CCN(c4ccc(C(=O)NC)nc4C(F)(F)F)CC3)ccc2c1=O] | 490.49 | 491.20 [M + H]⁺ | DMSO-d₆: δ 11.35 (s, 1H), 8.45-8.32 (m, 1H), 8.15 (d, J = 8.6 Hz, 1H), 8.00 (d, J = 8.6 Hz, 1H), 7.86 (d, J = 8.0 Hz, 1H), 7.20-7.07 (m, 2H), 3.95-3.80 (m, 2H), 3.57 (s, 2H), 3.05- 2.92 (m, 4H), 2.85-2.72 (m, 3H), 2.60-2.47 (m, 4H), 1.10 (t, J = 7.0 Hz, 3H). |
| 24 | 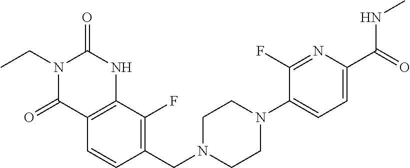 [SMILES: CCn1c(=O)[nH]c2c(F)c(CN3CCN(c4ccc(C(=O)NC)nc4F)CC3)ccc2c1=O] | 458.47 | 459.05 [M + H]⁺ | CDCl₃: δ 8.39 (s, 1H), 7.98 (d, J = 8.0 Hz, 1H), 7.89 (d, J = 8.1 Hz, 1H), 7.48 (d, J = 5.4 Hz, 1H), 7.32-7.27 (m, 2H), 4.13 (q, J = 6.9 Hz, 2H), 3.72 (s, 2H), 3.34-3.14 (m, 4H), 2.99 (d, J = 5.0 Hz, 3H), 2.77-2.52 (m, 4H), 1.30 (t, J = 7.1 Hz, 3H). |
| 25 | 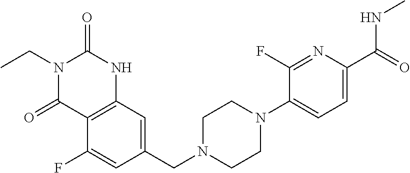 [SMILES: CCn1c(=O)[nH]c2cc(CN3CCN(c4ccc(C(=O)NC)nc4F)CC3)cc(F)c2c1=O] | 458.47 | 459.35 [M + H]⁺ | DMSO-d₆: δ 11.47 (s, 1H), 8.55-8.23 (m, 1H), 7.87 (dd, J = 20.9, 8.1 Hz, 1H), 7.57 (t, J = 9.3 Hz, 1H), 7.18 (s, 0.5H), 7.00 (s, 0.5H), 6.93 (d, J = 12.1 Hz, 1H), 4.02-3.78 (m, 2H), 3.59 (d, J = 8.8 Hz, 2H), 3.23-3.13 (m, 4H), 2.76 (d, J = 4.6 Hz, 3H), 2.60-2.53 (m, 4H), 1.13 (9, J = 6.5 Hz, 3H). |
| 26 | 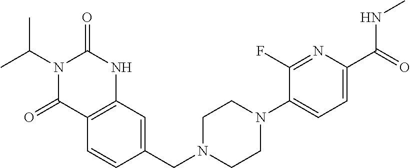 [SMILES: CNC(=O)c1ccc(N2CCN(Cc3ccc4c(=O)n(C(C)C)c(=O)[nH]c4c3)CC2)c(F)n1] | 454.51 | 455.40 [M + H]⁺ | CDCl₃: δ 8.17 (s, 1H), 8.07 (d, J = 8.3 Hz, 1H), 7.99 (d, J = 7.7 Hz, 1H), 7.49 (d, J = 5.2 Hz, 1H), 7.30 (dd, J = 10.3, 8.2 Hz, 1H), 7.19 (d, J = 8.6 Hz, 1H), 7.01 (s, 1H), 5.29 (dt, J = 13.4, 6.8 Hz, 1H), 3.62 (s, 2H), 3.25-3.21 (m, 4H), 2.99 (d, J = 5.0 Hz, 3H), 2.66-2.62 (m, 4H), 1.55 (s, 3H), 1.53 (s, 3H). |
| 27 | 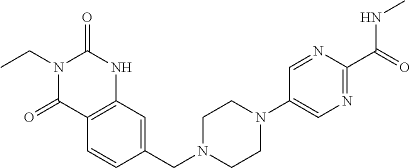 [SMILES: CCn1c(=O)[nH]c2cc(CN3CCN(c4cnc(C(=O)NC)nc4)CC3)ccc2c1=O] | 423.48 | 424.35 [M + H]⁺ | DMSO-d₆: δ 11.39 (s, 1H), 8.55 (d, J = 4.9 Hz, 1H), 8.52 (s, 2H), 7.91 (d, J = 8.4 Hz, 1H), 7.19 (d, J = 4.1 Hz, 2H), 3.93 (q, J = 6.9 Hz, 2H), 3.61 (s, 2H), 3.41 (d, J = 4.4 Hz, 4H), 2.78 (d, J = 4.7 Hz, 3H), 2.57-2.52 (m, 4H), 1.14 (t, J = 7.0 Hz, 3H). |
| 28 | 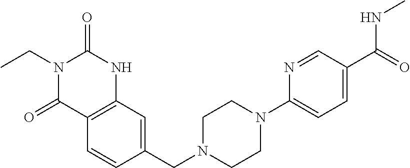 [SMILES: CCn1c(=O)[nH]c2cc(CN3CCN(c4ccc(C(=O)NC)cn4)CC3)ccc2c1=O] | 422.49 | 423.15 [M + H]⁺ | DMSO-d₆: δ 11.34 (s, 1H), 8.54 (d, J = 2.2 Hz, 1H), 8.20-8.15 (m, 1H), 7.91-7.85 (m, 2H), 7.15 (d, J = 6.6 Hz, 2H), 6.81 (d, J = 9.0 Hz, 1H), 3.90 (q, J = 7.0 Hz, 2H), 3.58-3.53 (m, 6H), 2.71 (d, J = 4.5 Hz, 3H), 2.45-2.42 (m, 4H), 1.11 (t, J = 7.0 Hz, 3H) |
| 29 | 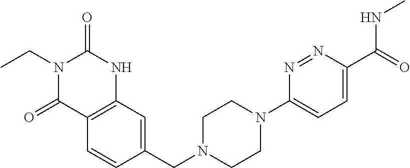 [SMILES: CCn1c(=O)[nH]c2cc(CN3CCN(c4ccc(C(=O)NC)nn4)CC3)ccc2c1=O] | 423.48 | 424.20 [M + H]⁺ | CDCl₃: δ 8.49 (s, 1H), 8,10 (d, J = 8.1 Hz, 1H), 8.02 (d. J = 9.5 Hz, 1H), 7.92 (d. J = 3.6 Hz, 1H), 7.22 (d, J = 8.0 Hz, 1H), 7.08 (s, 1H), 6.96 (d, J = 9.5 Hz, 1H), 4.13 (q, J = 7.0 Hz, 2H), 3.79-3.75 (m, 4H), 3.62 (s, 2H), 3.03 (d, J = 5.0 Hz, 3H), 2.63-2.59 (m, 4H), 1.26 (d, J = 10.3 Hz, 3H). |
| 30 | 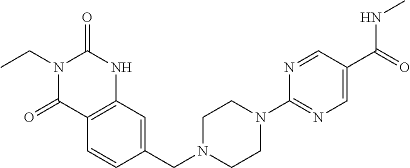 [SMILES: CCn1c(=O)[nH]c2cc(CN3CCN(c4ncc(C(=O)NC)cn4)CC3)ccc2c1=O] | 423.48 | 424.30 [M + H]⁺ | DMSO-d₆: δ 11.31 (s, 1H), 8.71 (s, 2H), 8.28 (d, J = 3.5 Hz, 1H), 7.87 (d, J = 8.3 Hz, 1H), 7.15 (s, 2H), 3.90 (dd, J = 12.9, 6.2 Hz, 2H), 3.83-3.74 (m, 4H), 3.55 (s, 2H), 2.72 (d, J = 4.0 Hz, 3H), 2.43-2.38 (m, 4H), 1.11 (s, 3H). |
| 31 | 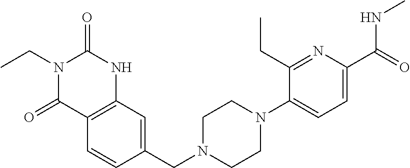 [SMILES: CCc1nc(C(=O)NC)ccc1N1CCN(Cc2ccc3c(=O)n(CC)c(=O)[nH]c3c2)CC1] | 450.54 | 451.40 [M + H]⁺ | DMSO-d₆: δ 11.38 (s, 1H), 8.41 (6, J = 5.9 Hz, 1H), 7.90 (d, J = 8.4 Hz, 1H), 7.79 (d, J = 8.4 Hz, 1H), 7.51 (d, J = 8.4 Hz, 1H), 7.19 (dd, J = 4.8, 1.9 Hz, 1H), 4.06-3.82 (m, 2H), 3.62 (s, 2H), 2.93 (s, 4H), 2.86-2.75 (m, 5H), 2.58 (s, 4H), 1.30 (t, J = 7.4 Hz, 3H), 1.14 (t, J = 6.9 Hz, 3H). |
| 32 | 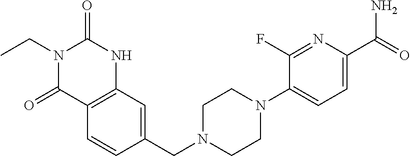 [SMILES: CCn1c(=O)[nH]c2cc(CN3CCN(c4ccc(C(N)=O)nc4F)CC3)ccc2c1=O] | 426.45 | 427.15 [M + H]⁺ | DMSO-d₆: δ 11.32 (s, 1H), 7.84 (dd, J = 18.0, 8.2 Hz, 2H), 7.74 (s, 1H), 7.53 (dd, J = 10.6, 8.1 Hz, 1H), 7.44 (s, 1H), 7.19-7.08 (m, 2H), 3.89 (q, J = 7.0 Hz, 2H), 3.57 (s, 2H), 3.21- 3.08 (m, 4H), 2.60-2.49 (m, 4H), 1.10 (t, J = 7.0 Hz, 3H). |
| 33 | 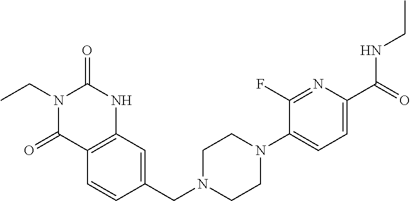 [SMILES: CCNC(=O)c1ccc(N2CCN(Cc3ccc4c(=O)n(CC)c(=O)[nH]c4c3)CC2)c(F)n1] | 454.51 | 455.20 [M + H]⁺ | DMSO-d₆: δ 11.38 (s, 1H), 8.45 (t, J = 6.0 Hz, 1H), 7.90 (d, J = 8.4 Hz, 1H), 7.85 (d, J = 8.0 Hz, 1H), 7.57 (dd, J = 10.1, 8.3 Hz, 1H), 7.18 (d, J = 4.3 Hz, 2H), 3.92 (q, J = 6.9 Hz, 2H), 3.60 (s, 2H), 3.27 (dd, J = 13.4, 6,9 Hz, 2H), 3.20-3.13 (m, 4H), 2.60-2.53 (m, 4H), 1.14 (t, J = 7.0 Hz, 3H), 1.08 (t, J = 7.1 Hz, 3H). |
| 34 | 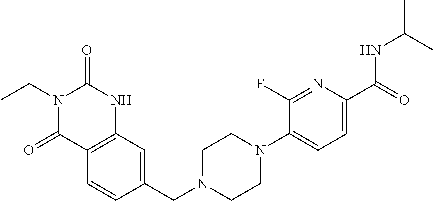 [SMILES: CCn1c(=O)[nH]c2cc(CN3CCN(c4ccc(C(=O)NC(C)C)nc4F)CC3)ccc2c1=O] | 468.53 | 469.40 [M + H]⁺ | DMSO-d₆: δ 11.35 (s, 1H), 8.04 (d, J = 8.4 Hz, 1H), 7.87 (d, J = 8.4 Hz, 1H), 7.81 (d, J = 8.0 Hz, 1H), 7.54 (dd, J = 10.6, 8.1 Hz, 1H), 7.22- 7.07 (m, 2H), 4.15-3.95 (m, 1H), 3.89 (q, J = 7.0 Hz, 2H), 3.57 (s, 2H), 3.17-3.09 (m, 4H), 2.57-2.49 (m, 4H), 1.17-1.04 (m, 9H). |
| 35 | 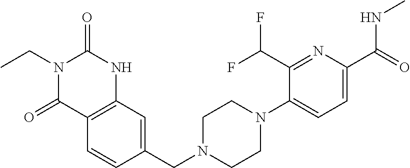 [SMILES: CCn1c(=O)[nH]c2cc(CN3CCN(c4ccc(C(=O)NC)nc4C(F)F)CC3)ccc2c1=O] | 472.50 | 473.15 [M + H]⁺ | DMSO-d₆: δ 11.35 (s, 1H), 8.37 (d, J = 5.0 Hz, 1H), 8.07 (d, J = 8.5 Hz, 1H), 7.85 (dd, J = 16.3, 8.3 Hz, 2H), 7.38-6.81 (m, 3H), 3.89 (q, J = 7.0 Hz, 2H), 3.59 (s, 2H), 3.04-2.93 (m, 4H), 2.80 (d, J = 4.8 Hz, 3H), 2.62-2.53 (m, 4H), 1.11 (t, J = 7.0 Hz, 3H). |
| 36 | 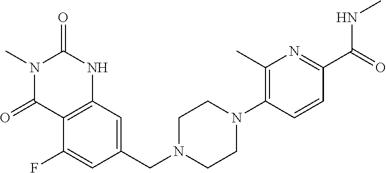 | 447.54 | 448.10 [M + H]⁺ | CDCl₃: δ 8.91 (s. 1H), 8.08 (d, J = 8.4 Hz, 1H), 7.96 (d, J = 8.7 Hz, 1H), 7.20 (d. J = 7.7 Hz, 1H), 7.12 (s, 1H), 6.79 (d, J = 7.5 Hz, 1H), 6.58 (s, 1H), 4.13 (q, J = 8.8, 8.2 Hz, 2H), 3.61 (s, 2H), 3.52 (t, J = 6.5 Hz, 2H), 3.36-3.22 (m, 4H), 3.14 (s, 3H), 2.93 (t, 2H), 2.65-2.47 (m, 4H), 1.29 (t, J = 7.0 Hz, 3H). |
| 37 | 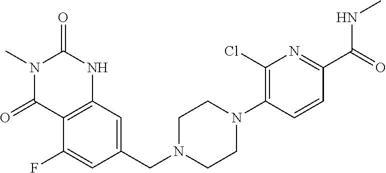 | 433.51 | 434.35 [M + H]⁺ | DMSO-d₆: δ 11.37 (s, 1H), 7.90 (d, J = 8.2 Hz, 1H), 7.65 (d, J = 8.7 Hz, 1H), 7.53 (s, 1H), 7.21- 7.14 (m, 2H), 6.89-6.81 (d, J = 8.0 Hz, 1H), 6.76 (s, 1H), 3.98-3.94 (m, 2H), 3.58 (s, 2H), 3.34-3.20 (m, 6H), 2.80 (t, J = 6.5 Hz, 2H), 2.58-2.37 (m, 4H), 1.14 (t. J = 7.0 Hz, 3H). |
| 38 | 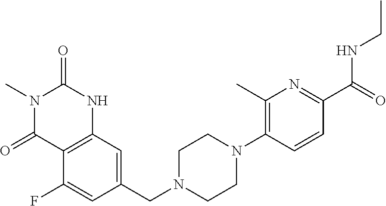 | 432.48 | 433.35 [M + H]⁺ | DMSO-d₆: δ 11.36 (s, 1H), 8.72 (d, J = 8.1 Hz, 1H), 8.04 (d, J = 6.2 Hz, 1H), 7.91 (d, J = 8.4 Hz, 1H), 7.25 (dd, J = 8.3, 2.7 Hz, 1H), 7.22- 7.15 (m, 2H), 6.72 (d, J = 2.7 Hz, 1H), 5.90 (d, J = 6.2 Hz, 1H), 3.93 (q, J = 6.9 Hz, 2H), 3.61 (s, 2H), 3,60-3.53 (m, 4H), 2.56-2.50 (m, 4H), 1.14 (t, J = 7.0 Hz, 3H). |
| 39 | 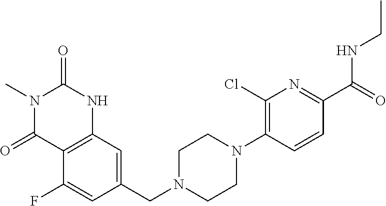 | 454.51 | 455.35 [M + H]⁺ | DMSO-d₆: δ 11.38 (s, 1H), 7.90 (d, J = 8.2 Hz, 1H), 7.62-7.41 (m, 2H), 7.18 (d. J = 5.4 Hz, 2H), 3.92 (q, J = 6.8 Hz, 2H), 3.60 (s, 2H), 3.19- 3.10 (m, 4H), 3.00 (s, 3H), 2.96 (s, 3H), 2.60- 2.53 (m, 4H), 1.14 (t, J = 7.0 Hz, 3H). |

### Example 40

### 5-(3-(3-methyl-2,4-dioxo-1,2,3,4-tetrahydroquinazolin-7-yl)benzamido)N-methylpicolinamide

(a) Preparation of N-methyl-5-(3-(4,4,5,5-tetramethyl-1,3,2-dioxaborolan-2-yl)benzamido)picolinamide: To a solution of 3-(4,4,5,5-tetramethyl-1,3,2-dioxaborolan-2-yl)benzoic acid (1.8 g, 7.15 mmol) in DCM (20 mL) was added oxalyl chloride (2.7 g, 21.45 mmol) and DMF (2 drops). The mixture was stirred at room temperature for 30 min. The solvent was evaporated, and the residue was dissolved in DCM (10 mL) for use. To a cooled solution of 5-amino-N-methylpicolinamide (0.9 g, 5.96 mmol) in DCM (20 mL) was added Et₃N (0.9 g, 8.94 mmol) and the above solution dropwise. The mixture was stirred at room temperature for 1 hour. After completion, the solvent was evaporated under reduced pressure and the residue was purified by column chromatography (ethyl acetate in petroleum ether, 0-50%) to afford the title compound (1.3 g, white solid, yield: 48%). MS (ESI, m/z): 381.90 [M+1]⁺.

(b) Preparation of 5-(3-(3-methyl-2,4-dioxo-1,2,3,4-tetrahydroquinazolin-7-yl)benzamido)N-methylpicolinamide: To a solution of N-methyl-5-(3-(4,4,5,5-tetramethyl-1,3,2-dioxaborolan-2-yl)benzamido)picolinamide (452.0 mg, 1.2 mmol) in DMF (4 mL) and H₂O (1 mL) was added 7-bromo-3-methylquinazoline-2,4(1H,3H)-dione (250.0 mg, 1.0 mmol), cesium carbonate (965.0 mg, 3.0 mmol), Pd(dppf)Cl₂ (74.0 mg, 0.1 mmol). The system was evacuated and backfilled with nitrogen three times and stirred at 100° C. overnight. After completion, the mixture was filtered, and the cake was washed with MeOH (10 mL) and DMF (10 mL). The solid was collected and dissolved with DMSO (10 mL). The insoluble was filtered off and the filtrate was dried to give the target compound (120.0 mg, white solid, yield: 28%).

The following compounds of Examples 41-47 were prepared using a synthesis method similar to that described in Example 40.

| Ex- | | | LC-MS | |
|---|---|---|---|---|
| ample | Compound | MW | (ESI) | ¹H NMR (400 MHz) |
| 40 | 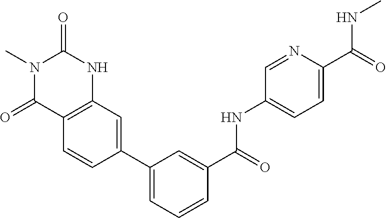 | 429.44 | 430.05 [M + H]⁺ | DMSO-d₆: δ 9.04 (d, J = 1.9 Hz, 1H), 8.70 (d, J = 4.8 Hz, 1H), 8.39 (dd, J = 8.5, 2.3 Hz, 1H), 8.28 (s, 1H), 8.07 (t, J = 6.2 Hz, 3H), 7.94 (d, J = 8.0 Hz, 1H), 7.73 (t, J = 7.7 Hz, 1H), 7.62 (d, J = 8.5 Hz, 1H), 7.49 (s, 1H), 3.29 (s, 3H), 2.82 (d, J = 4.7 Hz, 3H). |
| 41 | 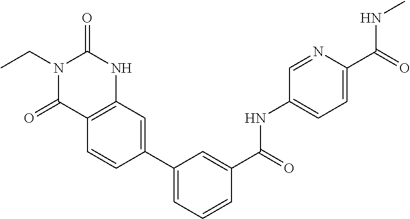 [SMILES: CCn1c(=O)[nH]c2cc(-c3cccc(C(=O)Nc4ccc(C(=O)NC)nc4)c3)ccc2c1=O] | 443.46 | 444.05 [M + H]⁺ | DMSO-d₆:δ 9.01 (d, J = 1.8 Hz, 1H), 8.69- 8.62 (m, 1H), 8.38 (dd, J = 8.6, 2.1 Hz, 1H), 8.30 (s, 1H), 8.01 (d, J = 14.4 Hz, 2H), 7.89 (t, J = 7.0 Hz, 2H), 7.63 (t, J = 7.8 Hz, 1H), 7.44 (s, 1H), 7.36 (d, J = 9.7 Hz, 1H), 3.91 (dd, J = 17.1, 3.7 Hz, 2H), 2.78 (d, J = 4.7 Hz, 3H), 1.13-1.06 (m, 3H). |
| 42 | 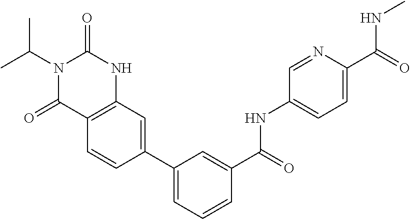 [SMILES: CNC(=O)c1ccc(NC(=O)c2cccc(-c3ccc4c(=O)n(C(C)C)c(=O)[nH]c4c3)c2)cn1] | 457.49 | 458.10 [M + H]⁺ | DMSO-d₆:δ 11.36 (s, 1H), 10.81 (s, 1H), 9.01 (d, J = 2.2 Hz, 1H), 8.69 (dd, J = 8.3, 3.7 Hz, 1H), 8.36 (dd, J = 8.5, 2.4 Hz, 1H), 8.24 (s, 1H), 8.10-7.97 (m, 3H), 7.91 (d, J = 8.0 Hz, 1H), 7.70 (t, J = 7.7 Hz, 1H), 7.58 (dd, J = 8.3, 1.5 Hz, 1H), 7.43 (d, J = 1.2 Hz, 1H), 5.24- 5.02 (m, 1H), 2.79 (d, J = 3.6 Hz, 3H), 1.43 (d, J = 6.9 Hz, 6H). |
| 43 | 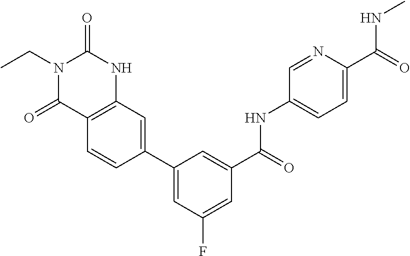 [SMILES: CCn1c(=O)[nH]c2cc(-c3cc(F)cc(C(=O)Nc4ccc(C(=O)NC)nc4)c3)ccc2c1=O] | 461.45 | 462.15 [M + H]⁺ | DMSO-d₆: δ 10.86 (s, 1H), 9.02 (d, J = 2.3 Hz, 1H), 8.70 (d, J = 5.1 Hz, 1H), 8.37 (dd, J = 8.4, 2.4 Hz, 1H), 8.13 (s, 1H), 8.07 (dd, J = 8.4, 2.4 Hz, 2H), 7.90 (d, J = 8.6 Hz, 1H), 7.84 (d, J = 9.8 Hz, 1H), 7.64 (dd, J = 8.2, 1.4 Hz, 1H), 7.49 (s, 1H), 3.96 (q, J = 7.3 Hz, 2H), 2.82 (d, J = 4.8 Hz, 3H), 1.17 (t, J = 7.0 Hz, 3H). |
| 44 | 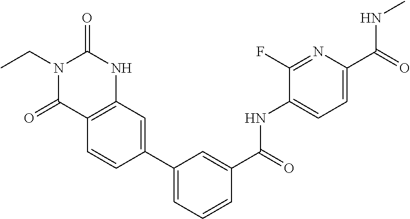 [SMILES: CCn1c(=O)[nH]c2cc(-c3cccc(C(=O)Nc4ccc(C(=O)NC)nc4F)c3)ccc2c1=O] | 461.45 | / | / |
| 45 | 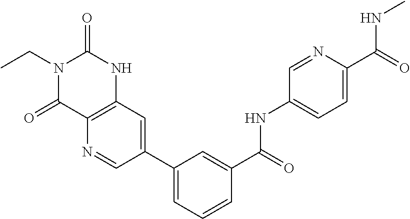 [SMILES: CCn1c(=O)[nH]c2cc(-c3cccc(C(=O)Nc4ccc(C(=O)NC)nc4)c3)cnc2c1=O] | 444.45 | 445.00 [M + H]⁺ | DMSO-d₆: δ 9.05 (s, 1H), 8.72 (d, J = 4.1 Hz, 1H), 8.48 (s, 1H), 8.43 (d, J = 10.4 Hz, 1H), 8.37 (s, 1H), 8.07-8.01 (m, 2H), 7.98 (d, J = 6.5 Hz, 1H), 7.74-7.64 (m, 2H), 3.93 (q, J = 7.2 Hz, 2H), 2.81 (s, 3H), 1.10 (t, J = 6.9 Hz, 3H). |
| 46 | 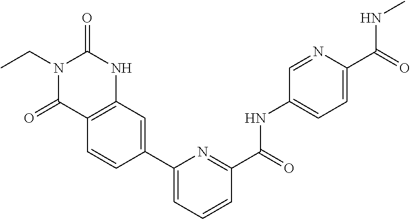 [SMILES: CCn1c(=O)[nH]c2cc(-c3cccc(C(=O)Nc4ccc(C(=O)NC)nc4)n3)ccc2c1=O] | 444.45 | 445.05 [M + H]⁺ | DMSO-d₆: δ 10.92 (s, 1H), 9.21-9.14 (m, 1H), 8.71 (dd, J = 8.2, 4.4 Hz, 1H), 8.49 (dd, J = 8.4, 2.3 Hz, 1H), 8.37-8.28 (m, 1H), 8.26 (t, J = 7.0 Hz, 2H), 8.21-8.16 (m, 2H), 8.09 (d, J = 8.4 Hz, 2H), 8.03 (s, 1H), 3.97 (q, J = 7.0 Hz, 2H), 2.83 (d, J = 4.7 Hz, 3H), 1.18 (t, J = 7.0 Hz, 3H). |
| 47 | 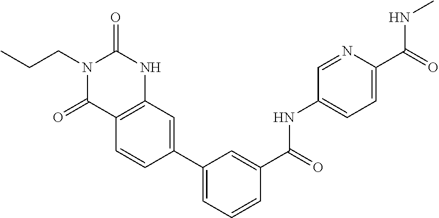 [SMILES: CCCn1c(=O)[nH]c2cc(-c3cccc(C(=O)Nc4ccc(C(=O)NC)nc4)c3)ccc2c1=O] | 457.49 | 458.10 [M + H]⁺ | DMSO-d₆: δ 11.50 (s, 1H), 10.82 (s, 1H), 9.04 (d, J = 2.4 Hz, 1H), 8.69 (q, J = 4.3 Hz, 1H), 8.38 (dd, J = 8.6, 2.4 Hz, 1H), 8.27 (s, 1H), 8.08 (s, 1H), 8.05 (s, 2H), 7.93 (d, J = 7.3 Hz, 1H), 7.73 (t, J = 7.7 Hz, 1H), 7.62 (dd, J = 8.3, 1.4 Hz, 1H), 7.48 (d. J = 1.1 Hz, 1H), 3.98- 3.74 (m, 2H), 2.82 (d, J = 4.8 Hz, 3H), 1.62 (dd, J = 14.7, 7.6 Hz, 2H), 0.90 (t, J = 7.5 Hz, 3H). |

The following compounds of Examples 48-113 were prepared using a synthesis method similar to that described in Example 12.

| Ex- | | | LC-MS | |
|---|---|---|---|---|
| ample | Compound | MW | (ESI) | ¹H NMR (400 MHz) |
| 48 | 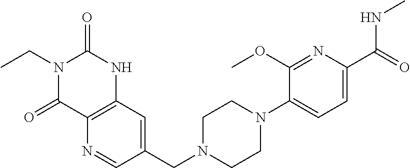 [SMILES: CCn1c(=O)[nH]c2cc(CN3CCN(c4ccc(C(=O)NC)nc4OC)CC3)cnc2c1=O] | 440.48 | / | / |
| 49 | 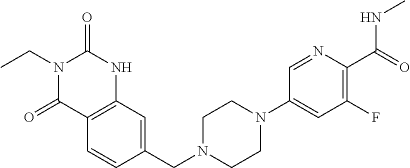 [SMILES: CCn1c(=O)[nH]c2cc(CN3CCN(c4cnc(C(=O)NC)c(F)c4)CC3)ccc2c1=O] | 440.48 | / | / |
| 50 | 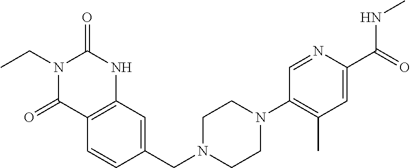 [SMILES: CCn1c(=O)[nH]c2cc(CN3CCN(c4cnc(C(=O)NC)cc4C)CC3)ccc2c1=O] | 436.52 | 437.15 [M + H]⁺ | DMSO-d₆: δ 8.54-8.44 (m, 1H), 8.16 (s, 1H), 7.87 (d, J = 8.4 Hz, 1H), 7.77 (s, 1H), 7.18- 7.10 (m, 2H), 3.90 (q, J = 7.1 Hz, 2H), 3.58 (s, 2H), 3.04-2.94 (m, 4H), 2.76 (d, J = 4.8 Hz, 3H), 2.60-2.50 (m, 4H), 2.27 (s, 3H), 1.11 (t, J = 7.0 Hz, 3H). |
| 51 | 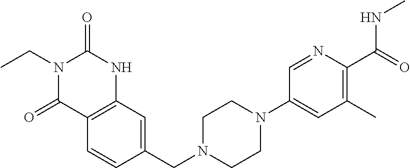 [SMILES: CCn1c(=O)[nH]c2cc(CN3CCN(c4cnc(C(=O)NC)c(C)c4)CC3)ccc2c1=O] | 436.52 | 437.35 [M + H]⁺ | CDCl₃:δ 9.14 (s, 1H), 8.09 (d, J = 8.1 Hz, 1H), 8.00 (d, J = 2.6 Hz, 1H), 7.96-7.88 (m, 1H), 7.21 (d, J = 8.2 Hz, 1H), 7.11 (s, 1H), 6.92 (d, J = 2.4 Hz, 1H), 4.14 (q, J = 7.0 Hz, 2H), 3.63 (s, 2H), 3.35-3.23 (m, 4H), 2.96 (d, J = 5.0 Hz, 3H), 2.71 (s. 3H), 2.65-2.55 (m, 4H), 1.30 (t, J = 7.0 Hz, 3H). |
| 52 | 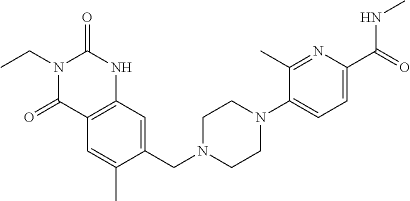 [SMILES: CCn1c(=O)[nH]c2cc(CN3CCN(c4ccc(C(=O)NC)nc4C)CC3)c(C)cc2c1=O] | 450.54 | 451.20 [M + H]⁺ | DMSO-d₆: δ 11.25 (s, 1H), 8.43-8.34 (m, 1H), 7.77 (d, J = 8.2 Hz, 1H), 7.69 (s, 1H), 7.45 (d, J = 8.3 Hz, 1H), 7.17 (s, 1H), 3.89 (q, J = 6.9 Hz, 2H), 3.54 (s, 2H), 3.02-2.84 (m, 4H), 2.77 (d, J = 4.6 Hz, 3H), 2.66-2.52 (m, 4H), 2.50 (s, 3H), 2.32 (s, 3H), 1.10 (t, J = 6.9 Hz, 3H) |
| 53 | 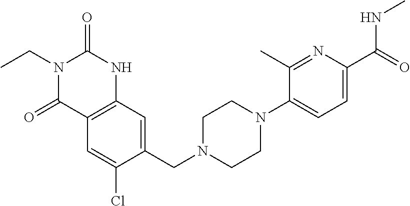 [SMILES: CCn1c(=O)[nH]c2cc(CN3CCN(c4ccc(C(=O)NC)nc4C)CC3)c(Cl)cc2c1=O] | 470.95 | 471.35 [M + H]⁺ | DMSO-d₆: δ 11.47 (s, 1H), 8.42 (s, 1H), 7.85 (s, 1H), 7.78 (d. J = 7.8 Hz, 1H), 7.46 (d, J = 8.4 Hz, 1H), 7.42 (s, 1H), 3.95-3.83 (m, 2H), 3.67 (s, 2H), 3.02-2.89 (m, 4H), 2.77 (d, J = 4.7 Hz, 3H), 2.70-2.55 (m, 4H), δ 2.47 (s, 3H), 1.10 (t, J = 6.8 Hz, 3H). |
| 54 | 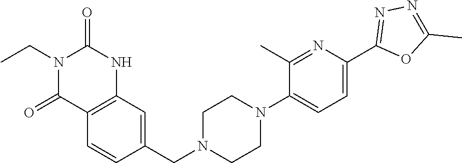 [SMILES: CCn1c(=O)[nH]c2cc(CN3CCN(c4ccc(-c5nnc(C)o5)nc4C)CC3)ccc2c1=O] | 461.53 | / | / |
| 55 | 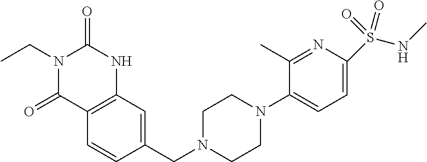 [SMILES: CCn1c(=O)[nH]c2cc(CN3CCN(c4ccc(S(=O)(=O)NC)nc4C)CC3)ccc2c1=O] | 472.56 | / | / |
| 56 | 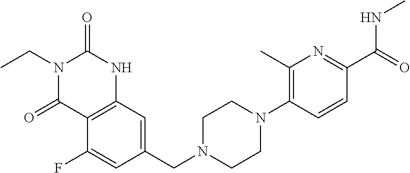 [SMILES: CCn1c(=O)[nH]c2cc(CN3CCN(c4ccc(C(=O)NC)nc4C)CC3)cc(F)c2c1=O] | 454.51 | 455.20 [M + H]⁺ | CDCl₃: δ 10.06-9.92 (m, 1H), 8.04-7.94 (m, 2H), 7.33 (d, J = 8.3 Hz, 1H), 7.04-6.98 (m, 1H), 6.92 (d, J = 11.4 Hz, 1H), 4.11 (q, J = 7.2 Hz, 2H), 3.63 (s, 2H), 3.07-2.99 (m, 7H), 2.75-2.60 (m, 4H), 2.51 (s, 3H), 1.29 (t, J = 7.1 Hz, 3H). |
| 57 | 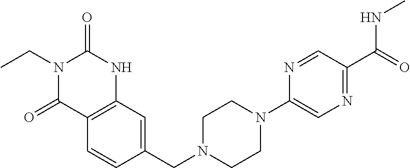 [SMILES: CCn1c(=O)[nH]c2cc(CN3CCN(c4cnc(C(=O)NC)cn4)CC3)ccc2c1=O] | 423.48 | 424.10 [M + H]⁺ | DMSO-d₆: δ 11.39 (s, 1H), 8.59 (s, 1H), 8.38- 8.32 (m, 1H), 8.26 (s, 1H), 7.90 (d, J = 7.8 Hz, 1H), 7.19 (s, 2H), 4.10-3.80 (m, 2H), 3.76- 3.64 (m, 4H), 3.60 (s, 2H), 3.33-3.26 (m, 4H), 2.77 (d, J = 4.5 Hz, 3H), 1.14 (t, J = 6.9 Hz, 3H). |
| 58 | 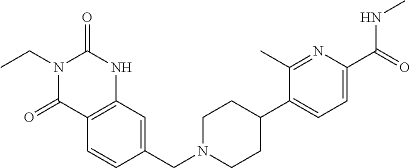 [SMILES: CCn1c(=O)[nH]c2cc(CN3CCC(c4ccc(C(=O)NC)nc4C)CC3)ccc2c1=O] | 435.53 | 436.40 [M + H]⁺ | CDCl₃:δ 8.07 (d, J = 8.0 Hz, 1H), 8.04-8.00 (m, 1H), 7.98 (d, J = 8.0 Hz, 1H), 7.64 (d, J = 8.1 Hz, 1H), 7.20 (d, J = 7.9 Hz, 1H), 7.10- 7.06 (m, 1H), 4.12 (q, J = 6.9 Hz, 2H), 3.60 (s, 2H), 3.06-2.93 (m, 5H), 2.79-2.66 (m, 1H), 2.56 (s, 3H), 2.22-2.10 (m, 2H), 1.81-1.70 (m, 4H), 1.28 (t, J = 7.1 Hz, 3H). |
| 59 | 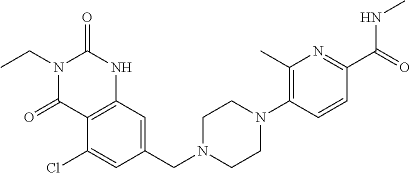 [SMILES: CCn1c(=O)[nH]c2cc(CN3CCN(c4ccc(C(=O)NC)nc4C)CC3)cc(Cl)c2c1=O] | 470.96 | 471.40 [M + H]⁺ | CDCl₃: δ 9.71 (s, 1H), 8.08-7.93 (m, 2H), 7.33 (d, J = 8.4 Hz, 1H), 7.25 (s, 1H), 7.10 (s, 1H), 4.12 (q, J = 7.1 Hz, 2H), 3.60 (s, 2H), 3.13-2.90 (m, 7H), 2.74-2.57 (m, 4H), 2.51 (s, 3H), 1.30 (t, J = 7.0 Hz, 3H). |
| 60 | 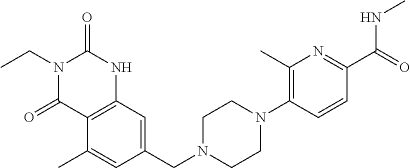 [SMILES: CCn1c(=O)[nH]c2cc(CN3CCN(c4ccc(C(=O)NC)nc4C)CC3)cc(C)c2c1=O] | 450.54 | / | / |
| 61 | 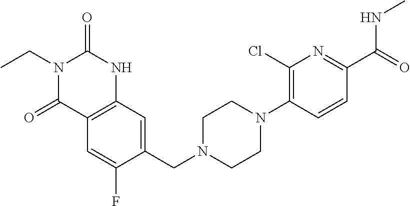 [SMILES: CCn1c(=O)[nH]c2cc(CN3CCN(c4ccc(C(=O)NC)nc4Cl)CC3)c(F)cc2c1=O] | 474.92 | 475.20 [M + H]⁺ | CDCl₃: δ 9.20 (s, 1H), 8.09 (d, J = 8.1 Hz, 1H), 7.79 (d, J = 9.4 Hz, 1H), 7.69 (s, 1H), 7.38 (d, J = 8.2 Hz, 1H), 7.23 (s, 1H), 4.13 (q, J = 9.0, 8.5 Hz, 2H), 3.73 (s, 2H), 3.25-3.12 (m, 4H), 3.02 (d, J = 5.1 Hz, 3H), 2.79-2.60 (m, 4H), 1.28 (q, J = 6.8 Hz, 3H). |
| 62 | 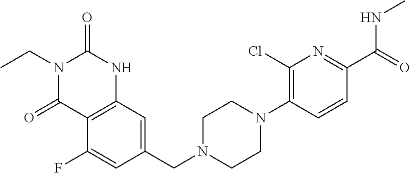 [SMILES: CCn1c(=O)[nH]c2cc(CN3CCN(c4ccc(C(=O)NC)nc4Cl)CC3)cc(F)c2c1=O] | 474.92 | 475.20 [M + H]⁺ | CD₃OD: δ 8.00-7.96 (m, 1H), 7.60 (dd, J = 8.0, 2.6 Hz, 1H), 7.24 (dd, J = 12.9, 12.3 Hz, 1H), 7.03-6.95 (m, 1H), 4.04 (q, J = 7.0 Hz, 2H), 3.65 (d, J = 15.3 Hz, 2H), 3.24-3.16 (m, 4H), 2.91 (s, 3H), 2.72-2.62 (m, 4H), 1.34- 1.30 (m, 3H). |
| 63 | 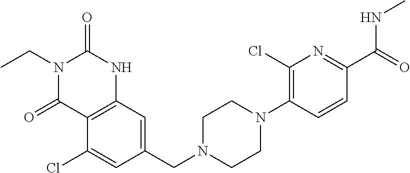 [SMILES: CCn1c(=O)[nH]c2cc(CN3CCN(c4ccc(C(=O)NC)nc4Cl)CC3)cc(Cl)c2c1=O] | 491.37 | 491.10 [M + H]⁺ | CDCl₃: δ 9.94 (s, 1H), 8.09 (d, J = 7.9 Hz, 1H), 7.69 (s, 1H), 7.40 (d, J = 7.2 Hz, 1H), 7.29 (s, 1H), 7.08 (s, 1H), 4.12 (d, J = 8.1 Hz, 2H), 3.63 (s, 2H), 3.29-3.15 (m, 4H), 3.02 (d, J = 4.1 Hz, 3H), 2.77-2.64 (m, 4H), 1.34-1.27 (m, 3H). |
| 64 | 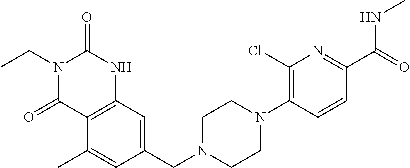 [SMILES: CCn1c(=O)[nH]c2cc(CN3CCN(c4ccc(C(=O)NC)nc4Cl)CC3)cc(C)c2c1=O] | 470.96 | / | / |
| 65 | 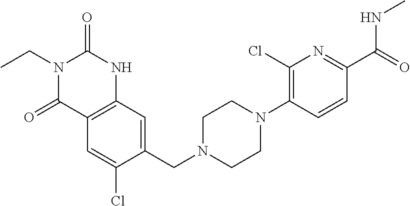 [SMILES: CCn1c(=O)[nH]c2cc(CN3CCN(c4ccc(C(=O)NC)nc4Cl)CC3)c(Cl)cc2c1=O] | 491.37 | 491.10 [M + H]⁺ | CDCl₃: δ 9.45 (s, 1H), 8.21-7.92 (m, 2H), 7.75-7.65 (m, 1H), 7.48-7.31 (m, 2H), 4.10 (q, J = 6.9 Hz, 2H), 3.74 (s, 2H), 3.24-3.12 (m, 4H), 3.01 (d, J = 5.0 Hz, 3H), 2.80-2.68 (m, 4H), 1.26 (t, J = 7.0 Hz, 3H). |
| 66 | 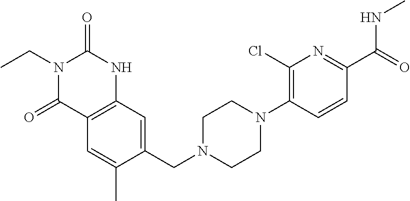 [SMILES: CCn1c(=O)[nH]c2cc(CN3CCN(c4ccc(C(=O)NC)nc4Cl)CC3)c(C)cc2c1=O] | 470.96 | 471.35 [M + H]⁺ | DMSO-d₆: δ 11.24 (s, 1H), 8.44-8.36 (m, 1H), 7.91 (d, J = 8.0 Hz, 1H), 7.69 (s, 1H), 7.63 (d, J = 8.2 Hz, 1H), 7.17 (s, 1H), 3.89 (q, J = 6.8 Hz, 2H), 3.54 (s, 2H), 3.14-3.0 (m, 4H), 2.76 (d, J = 4.7 Hz, 3H), 2.65-2.53 (m, 4H), 2.32 (s, 3H), 1.10 (t, J = 7.0 Hz, 3H). |
| 67 | 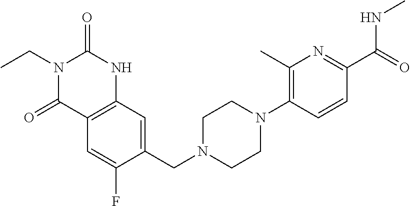 [SMILES: CCn1c(=O)[nH]c2cc(CN3CCN(c4ccc(C(=O)NC)nc4C)CC3)c(F)cc2c1=O] | 454.51 | 455.30 [M + H]⁺ | CDCl₃: δ 9.25 (s, 1H), 8,01 (d, J = 8.3 Hz, 2H), 7.78 (d, J = 9.5 Hz, 1H), 7.36-7.29 (m, 2H), 4.11 (q, 2H), 3.72 (s, 2H), 3.05 (d, J = 4.9 Hz, 3H), 3.05-2.96 (m, 4H), 2.79-2.61 (m, 4H), 2.51 (s, 3H), 1.29 (t, J = 7.2 Hz, 3H). |
| 68 | 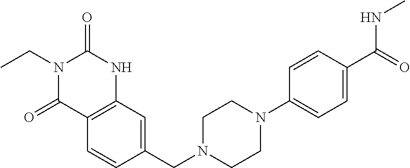 [SMILES: CCn1c(=O)[nH]c2cc(CN3CCN(c4ccc(C(=O)NC)cc4)CC3)ccc2c1=O] | 421.50 | 422.15 [M + H]⁺ | DMSO-d₆: δ 11.32 (s, 1H), 8.14-8.17 (m, 1H), 7.86 (d, J = 8.5 Hz, 1H), 7.66 (d, J = 8.9 Hz, 2H), 7.18-7.11 (m, 2H), 6.90 (d, J = 8.8 Hz, 2H), 3.89 (q, J = 7.0 Hz, 2H), 3.55 (s, 2H), 3.25-3.19 (m, 4H), 2.70 (d, J = 4.4 Hz, 3H), 2.51-2.48 (m, 4H), 1.11 (t, J = 7.0 Hz, 3H). |
| 69 | 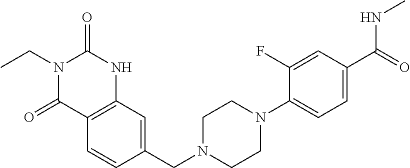 [SMILES: CCn1c(=O)[nH]c2cc(CN3CCN(c4ccc(C(=O)NC)cc4F)CC3)ccc2c1=O] | 439.49 | 440.15 [M + H]⁺ | DMSO-d₆: δ 11.33 (s, 1H), 8.34-8.27 (m, 1H), 7.87 (d, J = 8.3 Hz, 1H), 7.60-7.52 (m, 2H), 7.19-7.12 (m, 2H), 7.03 (t, J = 8.6 Hz, 1H), 3.89 (q, J = 6.8 Hz, 2H), 3.56 (s, 2H), 3.11-3.04 (m, 4H), 2.72 (d, J = 4.4 Hz, 4H), 2.56-2.49 (m, 3H), 1.11 (t, J = 7.0 Hz, 3H). |
| 70 | 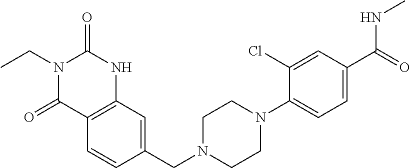 [SMILES: CCn1c(=O)[nH]c2cc(CN3CCN(c4ccc(C(=O)NC)cc4Cl)CC3)ccc2c1=O] | 455.94 | 456.15 [M + H]⁺ | DMSO-d₆:δ 11.33 (s, 1H), 8.41-8.33 (m, 1H), 7.87 (d, J = 8.4 Hz, 1H), 7.82 (d, J = 2.0 Hz, 1H), 7.73 (dd, J = 8.4, 2.0 Hz, 1H), 7.21- 7.08 (m, 3H), 3.89 (q, J = 7.0 Hz, 2H), 3.58 (s, 2H), 3.10-2.95 (m, 4H), 2.72 (d, J = 4.4 Hz, 3H), 2.60-2.50 (m, 4H), 1.11 (t, J = 7.0 Hz, 3H). |
| 71 | 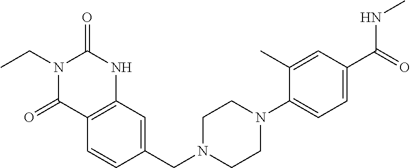 [SMILES: CCn1c(=O)[nH]c2cc(CN3CCN(c4ccc(C(=O)NC)cc4C)CC3)ccc2c1=O] | 435.53 | 436.10 [M + H]⁺ | DMSO-d₆: δ 11.36 (s, 1H), 8.22 (d, J = 4.5 Hz, 1H), 7.90 (d. J = 8.5 Hz, 1H), 7.62 (d, J = 11.0 Hz, 2H), 7.18 (d, J = 6.6 Hz, 2H), 7.02 (d, J = 8.2 Hz, 1H), 3.92 (q, J = 6.9 Hz, 2H), 3.60 (s. 2H), 2.94-2.83 (m, 4H), 2.74 (d, J = 4.5 Hz, 3H), 2.60-2.53 (m, 4H), 2.26 (s, 3H), 1.14 (t, J = 7.0 Hz, 3H). |
| 72 | 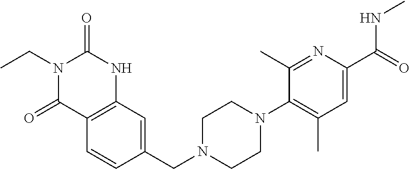 [SMILES: CCn1c(=O)[nH]c2cc(CN3CCN(c4c(C)cc(C(=O)NC)nc4C)CC3)ccc2c1=O] | 450.54 | 451.20 [M + H]⁺ | CDCl₃: δ 9.45 (s, 1H), 8.07 (d, J = 8.0 Hz, 1H), 8.02-7.93 (m, 1H), 7.80 (s, 1H), 7.21 (s, 1H), 7.19-7.11 (m, 1H), 4.13 (q, J = 7.0 Hz, 2H), 3.65 (s, 2H), 3.22-3.03 (m, 4H), 2.99 (d, J = 5.0 Hz, 3H), 2.68-2.54 (m, 4H), 2.51 (s, 3H), 2.35 (s, 3H), 1.28 (t, J = 7.0 Hz, 3H). |
| 73 | 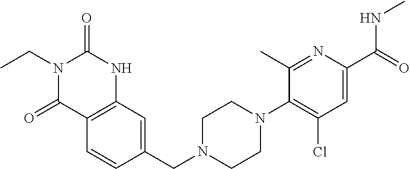 [SMILES: CCn1c(=O)[nH]c2cc(CN3CCN(c4c(Cl)cc(C(=O)NC)nc4C)CC3)ccc2c1=O] | 470.96 | / | / |
| 74 | 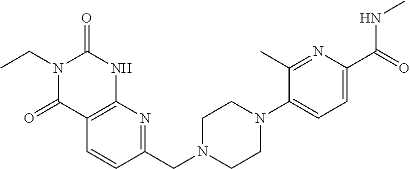 [SMILES: CCn1c(=O)[nH]c2nc(CN3CCN(c4ccc(C(=O)NC)nc4C)CC3)ccc2c1=O] | 437.50 | 438.30 [M + H]⁺ | DMSO-d₆: δ 11.91 (s, 1H), 8.46-8.40 (m, 1H), 8.29 (d, J = 5.1 Hz, 1H), 7.79 (d, J = 8.2 Hz, 1H), 7.49 (d, J = 8.1 Hz, 1H), 7.39 (d, J = 7.9 Hz, 1H), 3.91 (q, J = 6.7 Hz, 2H), 3.70 (s, 2H), 3.00-2.92 (m, 4H), 2.79 (d, J = 4.7 Hz, 3H), 2.68-2.58 (m, 4H), 2.48 (s, 3H), 1.13 (t, J = 7.0 Hz, 3H). |
| 75 | 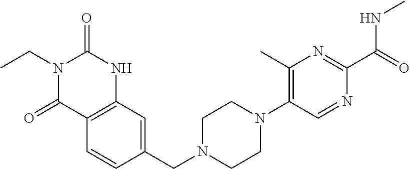 [SMILES: CCn1c(=O)[nH]c2cc(CN3CCN(c4cnc(C(=O)NC)nc4C)CC3)ccc2c1=O] | 437.50 | / | / |
| 76 | 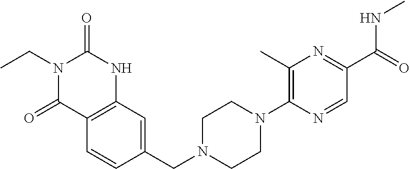 [SMILES: CCn1c(=O)[nH]c2cc(CN3CCN(c4ncc(C(=O)NC)nc4C)CC3)ccc2c1=O] | 437.50 | / | / |
| 77 | 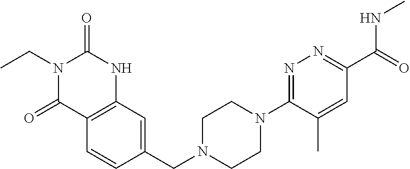 [SMILES: CCn1c(=O)[nH]c2cc(CN3CCN(c4nnc(C(=O)NC)cc4C)CC3)ccc2c1=O] | 437.50 | / | / |
| 78 | 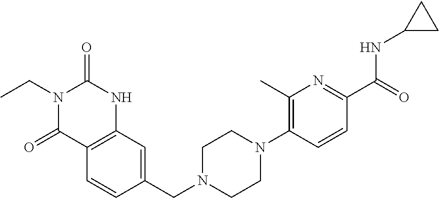 [SMILES: CCn1c(=O)[nH]c2cc(CN3CCN(c4ccc(C(=O)NC5CC5)nc4C)CC3)ccc2c1=O] | 462.55 | 463.45 [M + H]⁺ | CDCl₃: δ 9.22 (s, 1H), 8.09 (d, J = 8.1 Hz, 1H), 8.02-7.97 (m, 2H), 7.32 (d, J = 8.3 Hz, 1H), 7.21 (d, J = 7.9 Hz, 1H), 7.14 (s, 1H), 4.14 (q, J = 7.0 Hz, 2H), 3.65 (s, 2H), 3.02-2.95 (m, 4H), 2.94-2.89 (m, 1H), 2.70-2.61 (m, 4H), 2.49 (s, 3H), 1.30 (t, J = 7.0 Hz, 3H), 0.86 (q, J = 6.9 Hz, 2H), 0.68-0.61 (m, 2H). |
| 79 | 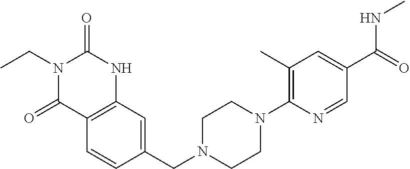 [SMILES: CCn1c(=O)[nH]c2cc(CN3CCN(c4ncc(C(=O)NC)cc4C)CC3)ccc2c1=O] | 436.52 | 437.20 [M + H]⁺ | CDCl₃: δ 9.41 (s, 1H), 8.46 (s, 1H), 8.06 (d, J = 8.2 Hz, 1H), 7.82 (s, 1H), 7.18 (d, J = 8.3 Hz, 1H), 7.13 (s, 1H), 6.26-6.14 (m, 1H), 4.18-4.06 (m, 2H), 3.61 (s, 2H), 3.30-3.20 (m, 4H), 2.99 (d, J = 4.7 Hz, 3H), 2.66-2.54 (m, 4H), 2.26 (s, 3H), 1.28 (t, J = 7.0 Hz, 3H), |
| 80 | 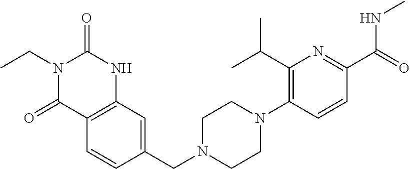 [SMILES: CCn1c(=O)[nH]c2cc(CN3CCN(c4ccc(C(=O)NC)nc4C(C)C)CC3)ccc2c1=O] | 464.57 | 465.25 [M + H]⁺ | DMSO-d₆: δ 11.35 (s, 1H), 8.45-8.37 (m, 1H), 7.87 (d, J = 8.6 Hz, 1H), 7.74 (d, J = 8.1 Hz, 1H), 7.53 (d, J = 8.4 Hz, 1H), 7.18-7.10 (m, 2H), 3.89 (q, J = 6.8 Hz, 2H), 3.59 (s, 2H), 3.48-3.39 (m, 1H), 2.91-2.83 (m, 4H), 2.80 (d, J = 4.9 Hz, 3H), 2.60-2.50 (m, 4H), 1.20 (d, J = 6.6 Hz, 6H), 1.11 (t, J = 7.0 Hz, 3H). |
| 81 | 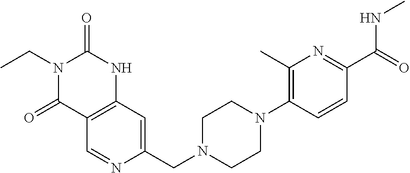 [SMILES: CCn1c(=O)[nH]c2cc(CN3CCN(c4ccc(C(=O)NC)nc4C)CC3)ncc2c1=O] | 437.50 | 438.20 [M + H]⁺ | CDCl₃: δ 9.16 (s, 1H), 8.19-8.10 (m, 1H), 8.07 (d. J = 8.1 Hz, 1H), 7.49 (s, 1H), 7.31 (d, J = 8.4 Hz, 1H), 4.13-4.04 (m, 2H), 3.81 (s, 2H), 3.10 (d, J = 5.1 Hz, 3H), 3.06-2.98 (m, 4H), 2.80-2.69 (m, 4H), 2.51 (s, 3H), 1.31- 1.26 (m, 3H). |
| 82 | 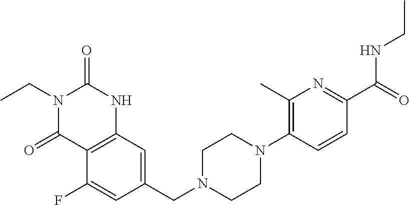 [SMILES: CCNC(=O)c1ccc(N2CCN(Cc3cc(F)c4c(=O)n(CC)c(=O)[nH]c4c3)CC2)c(C)n1] | 468.53 | 465.25 [M + H]⁺ | CDCl₃: δ 9.58 (s, 1H), 8.04-7.90 (m, 2H), 7.35 (d. J = 8.4 Hz, 1H), 7.12-7.00 (m, 1H), 6.91 (d, J = 11.1 Hz, 1H), 4.09 (q, J = 6.9 Hz, 2H), 3.68 (s, 2H), 3.56-3.38 (m, 2H), 3.16- 2.94 (m, 4H), 2.84-2.64 (m, 4H), 2.51 (s, 3H), 1.32-1.23 (m, 6H). |
| 83 | 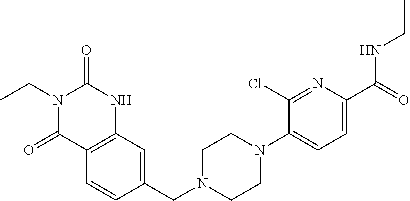 [SMILES: CCNC(=O)c1ccc(N2CCN(Cc3ccc4c(=O)n(CC)c(=O)[nH]c4c3)CC2)c(Cl)n1] | 470.96 | 471.20 [M + H]⁺ | DMSO-d₆: δ 11.33 (s, 1H), 8.43 (t, J = 6.1 Hz, 1H), 7.91 (d, J = 8.2 Hz, 1H), 7.87 (d, J = 8.4 Hz, 1H), 7.63 (d, J = 8.3 Hz, 1H), 7.18-7.13 (m, 2H), 3.89 (q, J = 7.0 Hz, 2H), 3.58 (s, 2H), 3.27-3.22 (m, 2H), 3.13-3.04 (m, 4H), 2.60- 2.51 (m, 4H), 1.04-1.03 (m, 6H). |
| 84 | 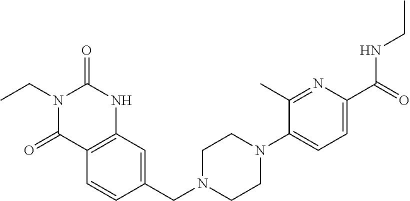 [SMILES: CCNC(=O)c1ccc(N2CCN(Cc3ccc4c(=O)n(CC)c(=O)[nH]c4c3)CC2)c(C)n1] | 450.54 | 451.20 [M + H]⁺ | CDCl₃: δ 10.00 (s, 1H), 8.09 (d, J = 8.0 Hz, 1H), 8.00 (d, J = 8.0 Hz, 2H), 7.32 (d, J = 8.3 Hz, 1H), 7.21 (d, J = 11.3 Hz, 2H), 4.15 (q, J = 6.9 Hz, 2H), 3.65 (s, 2H), 3.57-3.43 (m, 2H), 3.11-2.88 (m, 4H), 2.71-2.61 (m, 4H), 2.51 (s, 3H), 1.36-1.18 (m, 6H). |
| 85 | 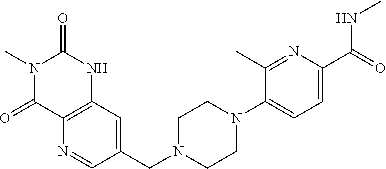 | 423.48 | / | / |
| 86 | 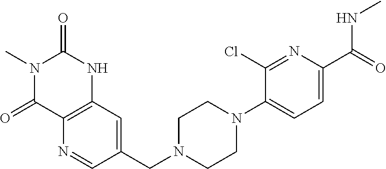 | 443.89 | / | / |
| 87 | 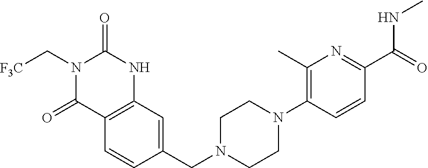 [SMILES: CNC(=O)c1ccc(N2CCN(Cc3ccc4c(=O)n(CC(F)(F)F)c(=O)[nH]c4c3)CC2)c(C)n1] | 490.49 | 491.45 [M + H]⁺ | DMSO-d₆: δ 8.42 (q, 1H), 8.31 (s, 1H), 7.94 (d, J = 8.4 Hz, 1H), 7.79 (d, J = 8.4 Hz, 1H), 7.48 (d, J = 8.4 Hz, 1H), 7.24 (d, J = 5.8 Hz, 2H), 4.72 (q, J = 10.1, 9.5 Hz, 2H), 3.64 (s, 3H), 3.16 (s, 2H), 3.03-2.90 (m, 4H), 2.80 (d, J = 4.8 Hz, 3H), 2.63-2.54 (m, 4H). |
| 88 | 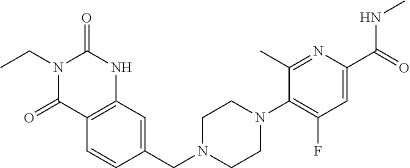 [SMILES: CCn1c(=O)[nH]c2cc(CN3CCN(c4c(F)cc(C(=O)NC)nc4C)CC3)ccc2c1=O] | 454.51 | / | / |
| 89 | 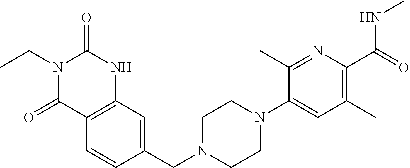 [SMILES: CCn1c(=O)[nH]c2cc(CN3CCN(c4cc(C)c(C(=O)NC)nc4C)CC3)ccc2c1=O] | 450.54 | / | / |
| 90 | 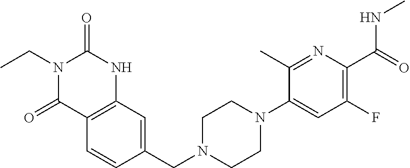 [SMILES: CCn1c(=O)[nH]c2cc(CN3CCN(c4cc(F)c(C(=O)NC)nc4C)CC3)ccc2c1=O] | 454.51 | / | / |
| 91 | 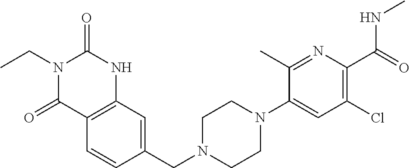 [SMILES: CCn1c(=O)[nH]c2cc(CN3CCN(c4cc(Cl)c(C(=O)NC)nc4C)CC3)ccc2c1=O] | 470.96 | / | / |
| 92 | 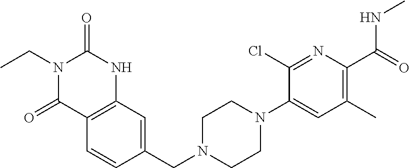 [SMILES: CCn1c(=O)[nH]c2cc(CN3CCN(c4cc(C)c(C(=O)NC)nc4Cl)CC3)ccc2c1=O] | 470.96 | / | / |
| 93 | 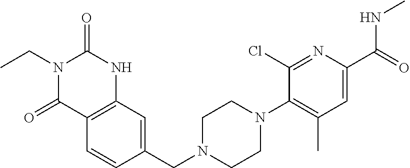 [SMILES: CCn1c(=O)[nH]c2cc(CN3CCN(c4c(C)cc(C(=O)NC)nc4Cl)CC3)ccc2c1=O] | 470.96 | / | / |
| 94 | 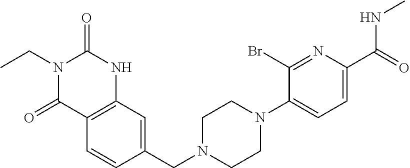 [SMILES: CCn1c(=O)[nH]c2cc(CN3CCN(c4ccc(C(=O)NC)nc4Br)CC3)ccc2c1=O] | 501.39 | 501.35 [M + H]⁺ | DMSO-d₆: δ 11.38 (s, 1H), 8.40 (q, 1H), 7.95 (d, J = 8.2 Hz, 1H), 7.90 (d, J = 8.5 Hz, 1H), 7.63 (d, J = 8.3 Hz, 1H), 7.18 (d, J = 7.6 Hz, 2H), 3.92 (q, J = 6.8, 6.1 Hz, 2H), 3.62 (s, 2H), 3.19-3.03 (m, 4H), 2.78 (d, J = 4.7 Hz, 3H), 2.63-2.56 (m, 4H), 1.13 (t, J = 7.0 Hz, 3H). |
| 95 | 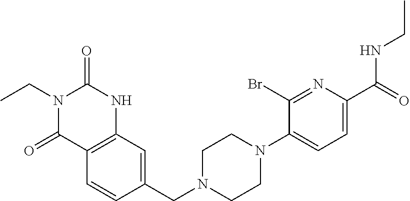 [SMILES: CCNC(=O)c1ccc(N2CCN(Cc3ccc4c(=O)n(CC)c(=O)[nH]c4c3)CC2)c(Br)n1] | 515.41 | 516.85 [M + H]⁺ | DMSO-d₆: δ 11.37 (s, 1H), 8.46 (t, J = 6.2 Hz, 1H), 7.95 (d, J = 8.1 Hz, 1H), 7.90 (d, J = 8.5 Hz, 1H), 7.63 (d, J = 8.2 Hz, 1H), 7.18 (d, J = 7.5 Hz, 2H), 3.92 (q, J = 6.8 Hz, 2H), 3.61 (s, 2H), 3.29 (t, J = 6.8 Hz, 2H), 3.13-3.03 (m, 4H), 2.65-2.55 (m, 4H), 1.19-1.00 (m, 6H). |
| 96 | 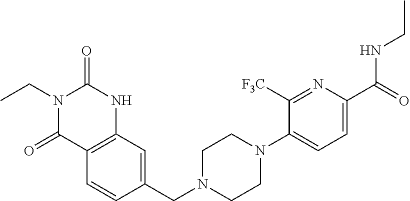 [SMILES: CCNC(=O)c1ccc(N2CCN(Cc3ccc4c(=O)n(CC)c(=O)[nH]c4c3)CC2)c(C(F)(F)F)n1] | 504.51 | / | / |
| 97 | 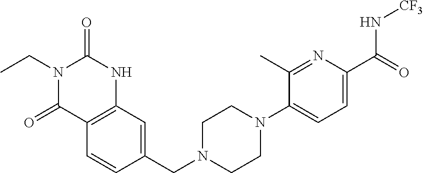 [SMILES: CCn1c(=O)[nH]c2cc(CN3CCN(c4ccc(C(=O)NC(F)(F)F)nc4C)CC3)ccc2c1=O] | 490.49 | / | / |
| 98 | 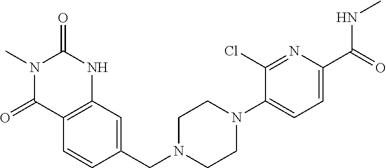 | 442.90 | 443.10 [M + H]⁺ | CDCl₃: δ 7.96 (d, J = 8.2 Hz, 1H), 7.93 (d, J = 8.2 Hz, 1H), 7.89-7.80 (m, 1H), 7.33 (d, J = 8.2 Hz, 1H), 7.13 (d, J = 8.0 Hz, 1H), 7.03 (s, 1H), 3.56 (s, 2H), 3.33 (s, 3H), 3.15-3.01 (m, 4H), 2.90 (d, J = 5.1 Hz, 3H), 2.64-2.54 (m, 4H). |
| 99 | 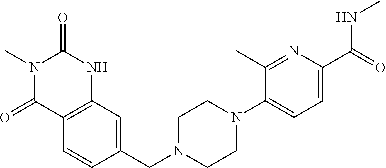 | 422.49 | 423.15 [M + H]⁺ | CDCl₃: δ 9.56 (s, 1H), 8.09 (d, J = 8.1 Hz, 1H), 8.03-7.95 (m, 2H), 7.33 (d, J = 8.3 Hz, 1H), 7.22 (d, J = 8.0 Hz, 1H), 7.18 (s, 1H), 3.65 (s, 2H), 3.48 (s, 3H), 3.03 (d, J = 5.1 Hz, 3H), 3.00-2.96 (m, 4H), 2.70-2.60 (m, 4H), 2.51 (s, 3H). |
| 100 | 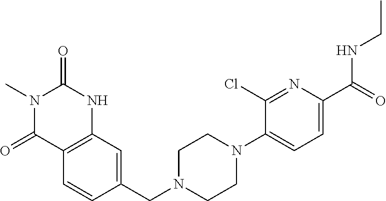 [SMILES: CCNC(=O)c1ccc(N2CCN(Cc3ccc4c(=O)n(C)c(=O)[nH]c4c3)CC2)c(Cl)n1] | 456.93 | 457.05 [M + H]⁺ | CDCl₃: δ 9.52 (s, 1H), 8.08 (d, J = 8.1 Hz, 1H), 8.03-7.93 (m, 2H), 7.31 (d, J = 8.3 Hz, 1H), 7.21 (d, J = 8.2 Hz, 1H), 7.15 (s, 1H), 3.64 (s, 2H), 3.53-3.47 (m, 2H), 3.46 (s, 3H), 3.02- 2.90 (m, 4H), 2.70-2.58 (m, 4H), 2.50 (s, 3H), 1.26 (t, J = 7.3 Hz, 3H). |
| 101 | 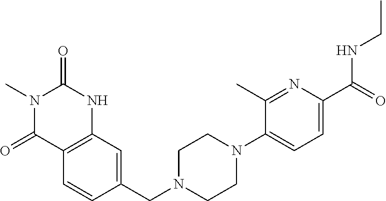 [SMILES: CCNC(=O)c1ccc(N2CCN(Cc3ccc4c(=O)n(C)c(=O)[nH]c4c3)CC2)c(C)n1] | 436.52 | 437.15 [M + H]⁺ | CDCl₃: δ 9.52 (s, 1H), 8.08 (d, J = 8.1 Hz, 1H), 8.03-7.93 (m, 2H), 7.31 (d. J = 8.3 Hz, 1H), 7.21 (d, J = 8.2 Hz, 1H), 7.15 (s, 1H), 3.64 (s, 2H), 3.53-3.47 (m, 2H), 3.46 (s, 3H), 3.02- 2.90 (m, 4H), 2.70-2.58 (m, 4H), 2.50 (s, 3H), 1.26 (t, J = 7.3 Hz, 3H). |
| 102 | 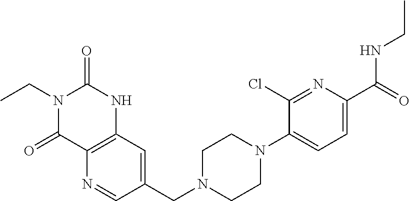 [SMILES: CCNC(=O)c1ccc(N2CCN(Cc3cnc4c(=O)n(CC)c(=O)[nH]c4c3)CC2)c(Cl)n1] | 471.95 | 472.35 [M + H]⁺ | CDCl₃: δ 8.51 (s, 1H), 7.99 (d, J = 8.2 Hz, 1H), 7.66 (s, 1H), 7.58 (d, J = 8.2 Hz, 1H), 4.10 (q, J = 7.0 Hz, 2H), 3.79 (s, 2H), 3.48-3.36 (m, 2H), 3.28-3.16 (m, 4H), 2.86-2.66 (m, 4H), 1.30-1.25 (m, 3H), 1.22 (t, J = 7.2 Hz, 3H). |
| 103 | 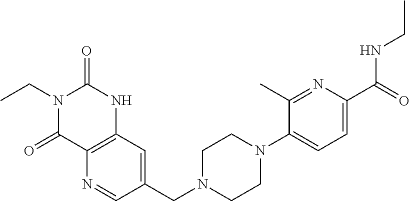 [SMILES: CCNC(=O)c1ccc(N2CCN(Cc3cnc4c(=O)n(CC)c(=O)[nH]c4c3)CC2)c(C)n1] | 451.53 | 452.45 [M + H]⁺ | DMSO-d₆: δ 8.47-8.40 (m, 2H), 7.77 (d, J = 8.2 Hz, 1H), 7.53 (s, 1H), 7.46 (d, J = 8.3 Hz, 1H), 3.90 (q, J = 6.8 Hz, 2H), 3.66 (s, 2H), 3.29-3.23 (m, 2H), 2.96-2.88 (m, 4H), 2.61- 2.52 (m, 4H), 2.46 (s, 3H), 1.12 (t. J = 7.0 Hz, 3H), 1.07 (t, J = 7.2 Hz, 3H). |
| 104 | 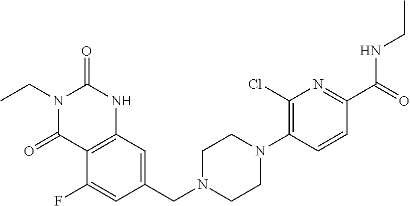 [SMILES: CCNC(=O)c1ccc(N2CCN(Cc3cc(F)c4c(=O)n(CC)c(=O)[nH]c4c3)CC2)c(Cl)n1] | 488.95 | 489.15 [M + H]⁺ | CDCl₃: δ 9.50 (s, 1H), 8.09 (d, J = 7.9 Hz, 1H), 7.68 (t, J = 6.0 Hz, 1H), 7.39 (d, J = 8.0 Hz, 1H), 6.96 (d, J = 11.7 Hz, 1H), 6.89 (s, 1H), 4.19-4.07 (m, 2H), 3.61 (s, 2H), 3.52-3.46 (m, 2H), 3.20-3.15 (m, 4H), 2.70-2.65 (m, 4H), 1.31-1.24 (m, 6H). |
| 105 | 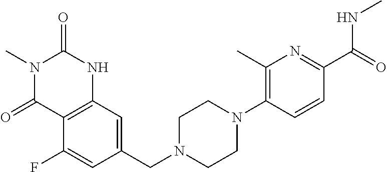 | 440.48 | 441.10 [M + H]⁺ | DMSO-d₆: δ 8.39 (d, J = 4.7 Hz, 1H), 7.86 (d, J = 8.4 Hz, 1H), 7.76 (d. J = 8.2 Hz, 1H), 7.45 (d, J = 8.2 Hz, 1H), 7.15 (s, 1H), 7.03-6.84 (m, 1H), 3.57 (d, J = 8.9 Hz, 2H), 3.19 (d, J = 20.1 Hz, 3H), 2.95-2.88 (m, 4H), 2.76 (d, J = 4.6 Hz, 3H), 2.58-2.52 (m, 4H), 2.46 (d, J = 5.5 Hz, 3H). |
| 106 | 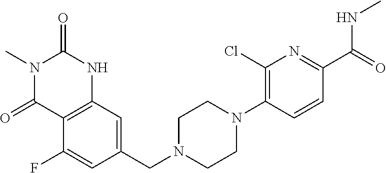 | 460.89 | 461.10 [M + H]⁺ | DMSO-d₆: δ 8.43 (d, J = 6.8 Hz, 1H), 8.30 (s, 1H), 7.94 (d, J = 8.1 Hz, 1H), 7.67 (d, J = 8.4 Hz, 1H), 7.01 (s, 1H), 6.94 (d, J = 11.9 Hz, 1H), 3.60 (s, 2H), 3.20 (s, 3H), 3.16-3.09 (m, 4H), 2.78 (d, J = 4.8 Hz, 3H), 2.61-2.56 (m, 4H). |
| 107 | 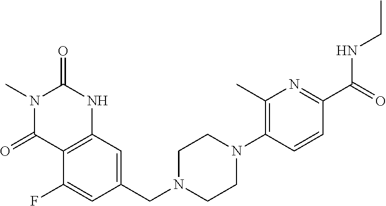 [SMILES: CCNC(=O)c1ccc(N2CCN(Cc3cc(F)c4c(=O)n(C)c(=O)[nH]c4c3)CC2)c(C)n1] | 454.51 | 455.35 [M + H]⁺ | DMSO-d₆: δ 11.53 (s, 1H), 8.45 (t, 1H), 7.80 (d, J = 8.0 Hz, 1H), 7.49 (d, J = 8.2 Hz, 1H), 7.01 (s, 1H), 6.94 (d, J = 11.9 Hz, 1H), 3.60 (s, 2H), 3.20 (s, 7H), 3.04-2.87 (m, 5H), 2.64- 2.55 (m, 4H), 1.11 (t, J = 7.0 Hz, 3H). |
| 108 | 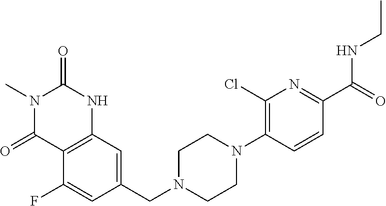 [SMILES: CCNC(=O)c1ccc(N2CCN(Cc3cc(F)c4c(=O)n(C)c(=O)[nH]c4c3)CC2)c(Cl)n1] | 474.92 | / | / |
| 109 | 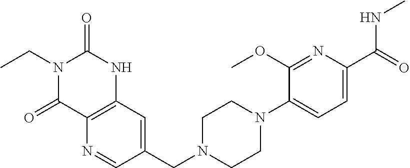 [SMILES: CCn1c(=O)[nH]c2cc(CN3CCN(c4ccc(C(=O)NC)nc4OC)CC3)cnc2c1=O] | 453.50 | 454.10 [M + H]⁺ | DMSO-d₆: δ 8.47 (s, 1H), 8.42 (s, 1H), 8.31 (d, J = 4.8 Hz, 1H), 7.54 (d, J = 8.7 Hz, 2H), 7.25 (d, J = 8.0 Hz, 1H), 3.99 (s, 3H), 3.94 (q, 2H), 3.66 (s, 2H), 3.13-3.04 (m, 4H), 2.80 (d, J = 4.7 Hz, 3H), 2.59-2.53 (m, 4H), 1.15 (t, J = 7.0 Hz, 3H). |
| 110 | 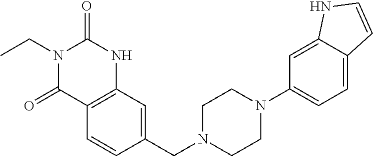 [SMILES: CCn1c(=O)[nH]c2cc(CN3CCN(c4ccc5cc[nH]c5c4)CC3)ccc2c1=O] | 403.49 | 404.40 [M + H]⁺ | DMSO-d₆: δ 11.37 (s, 1H), 10.74 (s, 1H), 7.90 (d, J = 7.9 Hz, 1H), 7.36 (d, J = 8.6 Hz, 1H), 7.24-7.16 (m, 2H), 7.17-7.08 (m, 1H), 6.83 (s, 1H), 6.77 (d, J = 8.7 Hz, 1H), 6.27 (s, 1H), 3.93 (q, J = 6.9 Hz, 2H), 3.60 (s, 2H), 3.15- 3.00 (m, 4H), 2.62-2.54 (m, 4H), 1.15 (t, J = 7.0 Hz, 3H). |
| 111 | 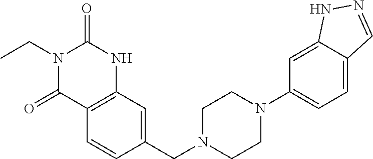 [SMILES: CCn1c(=O)[nH]c2cc(CN3CCN(c4ccc5cn[nH]c5c4)CC3)ccc2c1=O] | 404.47 | 405.10 [M + H]⁺ | DMSO-d₆:δ 12.62 (s, 1H), 11.36 (s, 1H), 7.99- 7.76 (m, 2H), 7.54 (d, J = 8.9 Hz, 1H), 7.18 (d, J = 8.5 Hz, 2H), 6.91 (d, J = 8.9 Hz, 1H), 6.77 (s, 1H), 3.92 (q, J = 6.7 Hz, 2H), 3.59 (s, 2H), 3.27-3.10 (m, 4H), 2.60-2.53 (m, 4H), 1.14 (t, J = 7.0 Hz, 3H). |
| 112 | 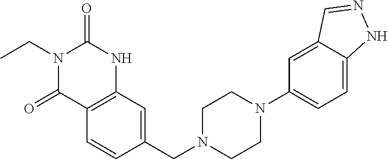 [SMILES: CCn1c(=O)[nH]c2cc(CN3CCN(c4ccc5[nH]ncc5c4)CC3)ccc2c1=O] | 404.47 | 405.25 [M + H]⁺ | CD₃OD: δ 8.00 (d, J = 8.1 Hz, 1H), 7.90 (s, 1H), 7.44 (d, J = 9.1 Hz, 1H), 7.29-7.19 (m, 4H), 4.05 (q, J = 6.1 Hz, 2H), 3.67 (s, 2H), 3.22-3.13 (m, 4H), 2.75-2.65 (m, 4H), 1.24 (t, J = 7.0 Hz, 3H). |
| 113 | 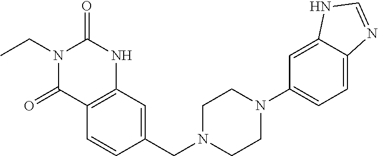 [SMILES: CCn1c(=O)[nH]c2cc(CN3CCN(c4ccc5nc[nH]c5c4)CC3)ccc2c1=O] | 404.47 | / | / |

The following compounds of Examples 114-122 were prepared using a synthesis method similar to that described in Example 15.

| Ex- | | | LC-MS | |
|---|---|---|---|---|
| ample | Compound | MW | (ESI) | ¹H NMR (400 MHz) |
| 114 | 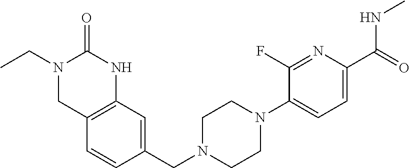 [SMILES: CCN1Cc2ccc(CN3CCN(c4ccc(C(=O)NC)nc4F)CC3)cc2NC1=O] | 426.50 | 427.10 [M + H]⁺ | CD₃OD:δ 7.89 (d, J = 8.0 Hz, 1H), 7.51 (dd, J = 10.2, 8.2 Hz, 1H), 7.09 (d, J = 7.6 Hz, 1H), 6.96 (d, J = 6.5 Hz, 1H), 6.82 (s, 1H), 4.49 (s, 2H), 3.60 (s, 2H), 3.46 (q, J = 7.1 Hz, 2H), 3.29- 3.20 (m, 4H), 2.91 (s, 3H), 2.77-2.57 (m, 4H), 1.20 (t, J = 7.1 Hz, 3H). |
| 115 | 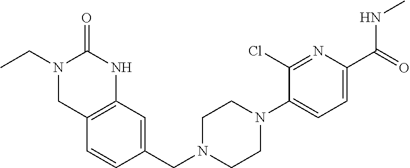 [SMILES: CCN1Cc2ccc(CN3CCN(c4ccc(C(=O)NC)nc4Cl)CC3)cc2NC1=O] | 442.95 | 443.05 [M + H]⁺ | CDCl₃: δ 8.06 (d, J = 8.1 Hz, 1H), 7.65 (s, 1H), 7.38 (d, J = 8.1 Hz, 1H), 7.00 (d, J = 7.7 Hz, 1H), 6.91 (d, J = 7.7 Hz, 1H), 6.87 (s, 1H), 6.69 (d, J = 1.5 Hz, 1H), 4.44 (s, 2H), 3.52 (s, 2H), 3.51-3.46 (m, 2H), 3.22-3.11 (m, 4H), 3.00 (d, J = 5.1 Hz, 3H), 2.72-2.57 (m, 4H), 1.23 (t, J = 7.2 Hz, 3H). |
| 116 | 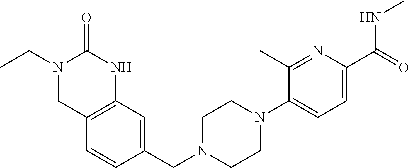 [SMILES: CCN1Cc2ccc(CN3CCN(c4ccc(C(=O)NC)nc4C)CC3)cc2NC1=O] | 422.53 | 423.15 [M + H]⁺ | CDCl₃: δ 8.11-7.81 (m, 2H), 7.33 (d, J = 8.3 Hz, 1H), 7.01 (d, J = 7.7 Hz, 1H), 6.92 (dd, J = 7.6, 1.5 Hz, 1H), 6.88 (s, 1H), 6.68 (s, 1H), 4.44 (s, 2H), 3.71-3.37 (m, 4H), 3.12-2.88 (m, 7H), 2.74-2.56 (m, 4H), 2.50 (s, 3H), 1.30-1.25 (m, 3H). |
| 117 | 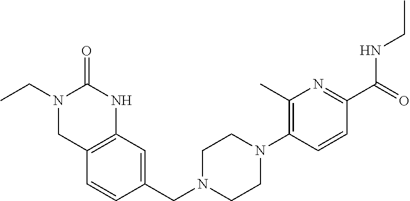 [SMILES: CCNC(=O)c1ccc(N2CCN(Cc3ccc4c(c3)NC(=O)N(CC)C4)CC2)c(C)n1] | 436.56 | 437.15 [M + H]⁺ | CDCl₃: δ 8.06-7.74 (m, 2H), 7.33 (d, J = 8.3 Hz, 1H), 7.01 (d, J = 7.7 Hz, 2H), 6.92 (d, J = 7.4 Hz, 1H), 6.70 (s, 1H), 4.44 (s, 2H), 3.73- 3.29 (m, 6H), 3.07-2.91 (m, 4H), 2.70-2.57 (m, 4H), 2.51 (s, 3H), 1.33-1.08 (m, 6H). |
| 118 | 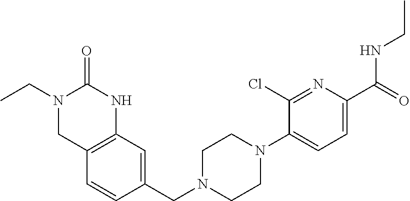 [SMILES: CCNC(=O)c1ccc(N2CCN(Cc3ccc4c(c3)NC(=O)N(CC)C4)CC2)c(Cl)n1] | 456.98 | 457.15 [M + H]⁺ | CDCl₃:δ 8.07 (d, J = 8.1 Hz, 1H), 7.66 (t, J = 5.3 Hz, 1H), 7.39 (d, J = 8.2 Hz, 1H), 7.15 (s, 1H), 7.01 (d, J = 7.7 Hz, 1H), 6.93 (d, J = 7.7 Hz, 1H), 6.73 (s, 1H), 4.44 (s, 2H), 3.61-3.37 (m, 6H), 3.23-3.05 (m, 4H), 2.82-2.58 (m, 4H), 1.36-1.09 (m, 6H). |
| 119 | 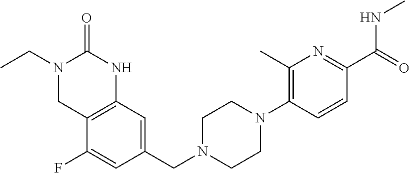 [SMILES: CCN1Cc2c(F)cc(CN3CCN(c4ccc(C(=O)NC)nc4C)CC3)cc2NC1=O] | 440.52 | 441.25 [M + H]⁺ | CDCl₃:δ 8.00-7.91 (m, 2H), 7.33 (d, J = 8.2 Hz, 1H), 7.15 (s, 1H), 6.69 (d, J = 10.3 Hz, 1H), 6.47 (s, 1H), 4.47 (s, 2H), 3.55-3.49 (m, 2H), 3.49 (s, 3H), 3.01 (d, J = 5.1 Hz, 2H), 3.00- 2.95 (m, 4H), 2.66-2.58 (m, 4H), 2.50 (s, 3H), 1.23 (t, J = 8.0 Hz, 3H). |
| 120 | 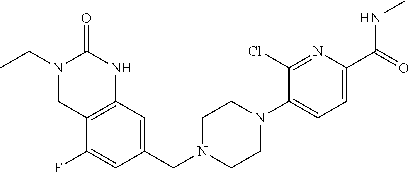 [SMILES: CCN1Cc2c(F)cc(CN3CCN(c4ccc(C(=O)NC)nc4Cl)CC3)cc2NC1=O] | 460.94 | 461.15 [M + H]⁺ | CDCl₃: δ 8.09 (d, J = 8.0 Hz, 1H), 7.68 (d, J = 4.9 Hz, 1H), 7.41 (d, J = 8.1 Hz, 1H), 7.10 (s, 1H, 6.72 (d, J = 9.7 Hz, 1H), 6.52 (s, 1H), 4.49 (s, 2H), 3.67-3.43 (m, 4H), 3.29-3.16 (m, 4H), 3.02 (d, J = 5.0 Hz, 3H), 2.77-2.61 (m, 4H), 1.25 (t, J = 7.3 Hz, 3H). |
| 121 | 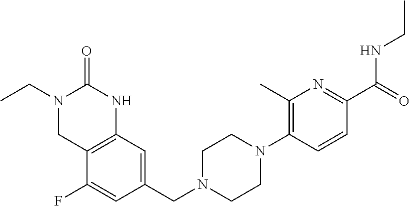 [SMILES: CCNC(=O)c1ccc(N2CCN(Cc3cc(F)c4c(c3)NC(=O)N(CC)C4)CC2)c(C)n1] | 454.55 | 455.25 [M + H]⁺ | DMSO-d₆: δ 9.32 (s, 1H), 8.42 (t, J = 6.2 Hz, 1H), 7.75 (s, 1H), 7.45 (d, J = 8.3 Hz, 1H), 6.65 (d, J = 10.1 Hz, 1H), 6.57 (s, 1H), 4.40 (s, 2H), 3.45-3.40 (m, 4H), 3.27-3.22 (m, 6H), 2.95- 2.86 (m, 4H), 2.54-2.49 (m, 3H), 1.12-1.01 (m, 6H). |
| 122 | 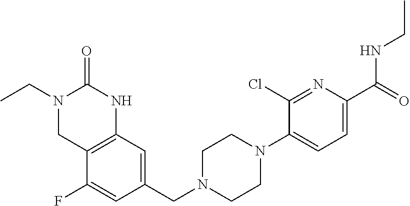 [SMILES: CCNC(=O)c1ccc(N2CCN(Cc3cc(F)c4c(c3)NC(=O)N(CC)C4)CC2)c(Cl)n1] | 474.97 | 475.20 [M + H]⁺ | CDCl₃: δ 8.07 (d, J = 8.0 Hz, 1H), 7.67 (t, J = 4.5 Hz, 1H), 7.39 (d, J = 8.0 Hz, 1H), 6.94 (s, 1H), 6.69 (d, J = 9.8 Hz, 1H), 6.46 (s, 1H), 4.47 (s, 2H), 3.55-3.43 (m, 6H), 3.24-3.11 (m, 4H), 2.71-2.58 (m, 4H), 1.27-1.21 (m, 6H). |
| 123 | 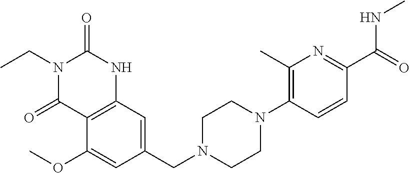 [SMILES: CCn1c(=O)[nH]c2cc(CN3CCN(c4ccc(C(=O)NC)nc4C)CC3)cc(OC)c2c1=O] | 466.54 | / | / |
| 124 | 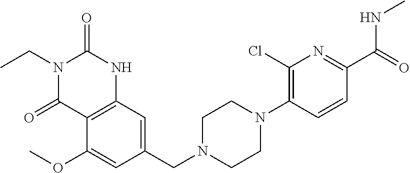 [SMILES: CCn1c(=O)[nH]c2cc(CN3CCN(c4ccc(C(=O)NC)nc4Cl)CC3)cc(OC)c2c1=O] | 486.96 | / | / |
| 125 | 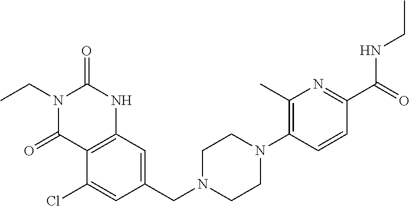 [SMILES: CCNC(=O)c1ccc(N2CCN(Cc3cc(Cl)c4c(=O)n(CC)c(=O)[nH]c4c3)CC2)c(C)n1] | 484.99 | / | / |
| 126 | 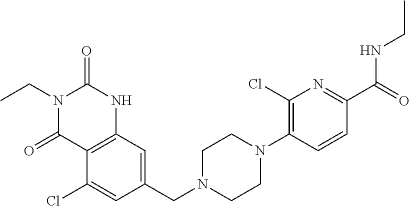 [SMILES: CCNC(=O)c1ccc(N2CCN(Cc3cc(Cl)c4c(=O)n(CC)c(=O)[nH]c4c3)CC2)c(Cl)n1] | 505.40 | 505.10 [M + H]⁺ | CDCl₃:δ 9.96 (s, 1H), 8.10 (d, J = 8.1 Hz, 1H), 7.69 (t, 1H), 7.39 (d, J = 7.9 Hz, 1H), 7.28 (s, 1H), 7.04 (s, 1H), 4.13 (q, J = 6.6 Hz, 2H), 3.61 (s, 2H), 3.55-3.39 (m, 2H), 3.24-3.02 (m, 4H), 2.80-2.51 (m, 4H), 1.40-1.09 (m, 6H). |
| 127 | 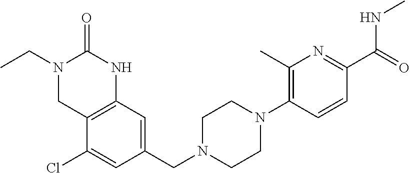 [SMILES: CCN1Cc2c(Cl)cc(CN3CCN(c4ccc(C(=O)NC)nc4C)CC3)cc2NC1=O] | 456.98 | / | / |
| 128 | 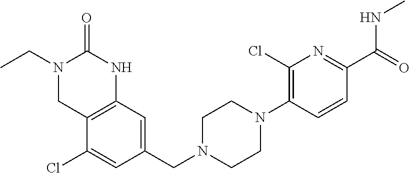 [SMILES: CCN1Cc2c(Cl)cc(CN3CCN(c4ccc(C(=O)NC)nc4Cl)CC3)cc2NC1=O] | 477.39 | / | / |
| 129 | 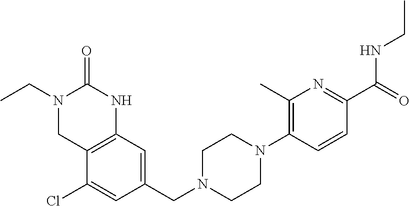 [SMILES: CCNC(=O)c1ccc(N2CCN(Cc3cc(Cl)c4c(c3)NC(=O)N(CC)C4)CC2)c(C)n1] | 471.00 | / | / |
| 130 | 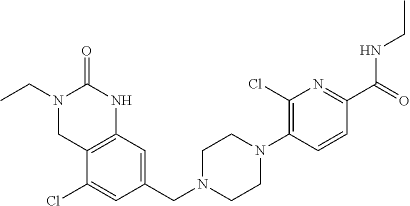 [SMILES: CCNC(=O)c1ccc(N2CCN(Cc3cc(Cl)c4c(c3)NC(=O)N(CC)C4)CC2)c(Cl)n1] | 491.42 | / | / |
| 131 | 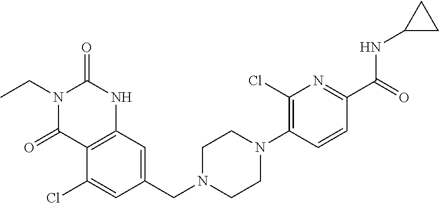 [SMILES: CCn1c(=O)[nH]c2cc(CN3CCN(c4ccc(C(=O)NC5CC5)nc4Cl)CC3)cc(Cl)c2c1=O] | 517.41 | 517.10 [M + H]⁺ | CDCl₃:δ 9.33 (s, 1H), 8.09 (d, J = 8.0 Hz, 1H), 7.71 (s, 1H), 7.39 (d, J = 8.4 Hz, 1H), 7.28 (s, 1H), 7.01 (s, 1H), 4.12 (q, J = 7.2, 6.7 Hz, 2H), 3.60 (s, 2H), 3.23-3.09 (m, 4H), 2.99-2.84 (m, 1H), 2.73-2.60 (m, 4H), 1.30 (t, J = 6.9 Hz, 3H), 0.87 (d, J = 6.2 Hz, 2H), 0.66 (s, 2H). |
| 132 | 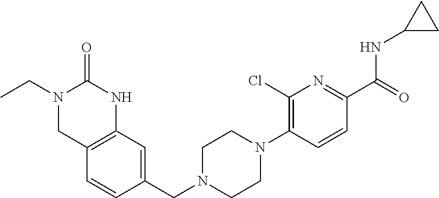 [SMILES: CCN1Cc2ccc(CN3CCN(c4ccc(C(=O)NC5CC5)nc4Cl)CC3)cc2NC1=O] | 468.98 | 469.20 [M + H]⁺ | CDCl₃:δ 8.05 (d, J = 8.1 Hz, 1H), 7.67 (s, 1H), 7.36 (d, J = 8.2 Hz, 1H), 7.00 (d, J = 7.6 Hz, 1H), 6.91 (s, 1H), 6.77 (s, 1H), 6.68 (s, 1H), 4.42 (s, 2H), 3.67-3.38 (m, 4H), 3.27-3.08 (m, 4H), 2.95-2.79 (m, 1H), 2.73-2.57 (m, 4H), 1.20 (t, J = 7.1 Hz, 3H), 0.84 (d, J = 6.8 Hz, 2H), 0.64 (s, 2H). |
| 133 | 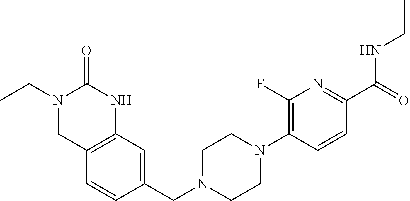 [SMILES: CCNC(=O)c1ccc(N2CCN(Cc3ccc4c(c3)NC(=O)N(CC)C4)CC2)c(F)n1] | 440.52 | / | / |
| 134 | 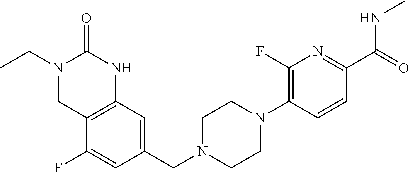 [SMILES: CCN1Cc2c(F)cc(CN3CCN(c4ccc(C(=O)NC)nc4F)CC3)cc2NC1=O] | 444.49 | 445.4 [M + H]⁺ | CDCl₃: δ 7.99 (d, J = 8.2 Hz, 1H), 7.59-7.42 (m, 1H), 7.30 (dd, J = 10.2, 8.0 Hz, 1H), 7.03 (s, 1H), 6.69 (d, J = 9.1 Hz, 1H), 6.46 (s, 1H), 4.47 (s, 2H), 3.73-3.42 (m, 4H), 3.34-3.17 (m. 4H), 2.99 (d, J = 5.0 Hz, 3H), 2.70-2.28 (m, 4H), 1.23 (t, J = 7.2 Hz, 3H). |
| 135 | 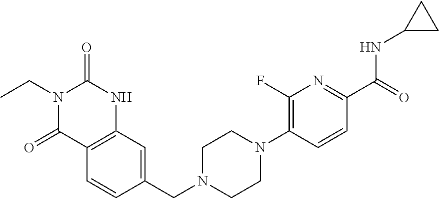 [SMILES: CCn1c(=O)[nH]c2cc(CN3CCN(c4ccc(C(=O)NC5CC5)nc4F)CC3)ccc2c1=O] | 466.51 | 467.45 [M + H]⁺ | CDCl₃: δ 9.25 (s, 1H), 8.09 (d, J = 8.2 Hz, 1H), 8.00 (d, J = 8.0 Hz, 1H), 7.53 (s, 1H), 7.30 (d, J = 9.4 Hz, 1H), 7.22 (d, J = 8.2 Hz, 1H), 7.12 (s, 1H), 4.14 (q, J = 6.9 Hz, 2H), 3.65 (s, 2H), 3.28- 3.15 (m, 4H), 2.98-2.79 (m, 1H), 2.77-2.53 (m, 4H), 1.30 (t, J = 7.0 Hz, 3H), 0.85 (q, J = 6.5 Hz, 2H), 0.63 (s, 2H). |
| 136 | 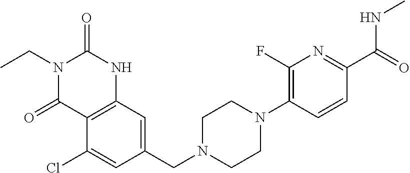 [SMILES: CCn1c(=O)[nH]c2cc(CN3CCN(c4ccc(C(=O)NC)nc4F)CC3)cc(Cl)c2c1=O] | 474.92 | 475.14 [M + H]⁺ | CDCl₃: δ 9.54 (s, 1H), 8.01 (d, J = 8.1 Hz, 1H), 7.52 (q, J = 5.3 Hz, 1H), 7.33 (d, J = 9.7 Hz, 1H), 7.29 (s, 1H), 7.04 (s, 1H), 4.25-3.91 (m, 2H), 3.60 (s, 2H), 3.34-3.19 (m, 4H), 3.01 (d, J = 5.0 Hz, 3H), 2.77-2.52 (m, 4H), 1.30 (t, J = 7.1 Hz, 3H). |
| 137 | 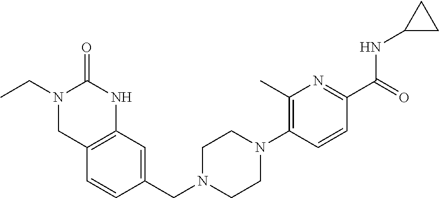 [SMILES: CCN1Cc2ccc(CN3CCN(c4ccc(C(=O)NC5CC5)nc4C)CC3)cc2NC1=O] | 448.57 | 449.25 [M + H]⁺ | CD₃OD: δ 7.86 (d, J = 8.4 Hz, 1H), 7.40 (d, J = 8.3 Hz, 1H), 7.03 (d, J = 7.6 Hz, 1H), 6.92 (d, J = 7.6 Hz, 1H), 6.77 (s, 1H), 4.47 (s, 2H), 3.55 (s, 2H), 3.44 (q, J = 7.1 Hz, 2H), 3.09-2.93 (m, 4H), 2.88-2.76 (m, 1H), 2.70-2.61 (m, 4H), 2.49 (s, 3H), 1.19 (t, J = 7.2 Hz, 3H), 0.91-0.73 (m, 2H), 0.71-0.50 (m, 2H). |
| 138 | 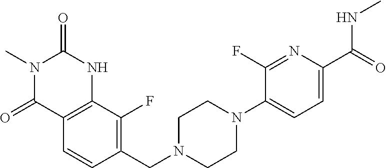 | 444.44 | 445.15 [M + H]⁺ | DMSO-d₆: δ 11.61 (s, 1H), 8.43 (d, J = 5.2 Hz, 1H), 7.86-7.79 (m, 1H), 7.75 (d, J = 8.1 Hz, 1H), 7.56 (dd, J = 10.7, 8.1 Hz, 1H), 7.25 (dd, J = 8.2, 6.0 Hz, 1H), 3.68 (s, 2H), 3.26 (s, 3H), 3.16 (t, J = 4.8 Hz, 4H), 2.75 (d, J = 4.7 Hz, 3H), 2.58 (t, J = 4.5 Hz, 4H). |
| 139 | 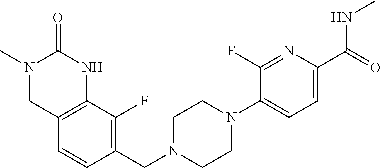 | 430.46 | / | / |
| 140 | 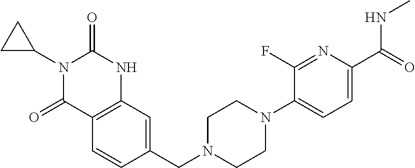 [SMILES: CNC(=O)c1ccc(N2CCN(Cc3ccc4c(=O)n(C5CC5)c(=O)[nH]c4c3)CC2)c(F)n1] | 452.49 | / | / |
| 141 | 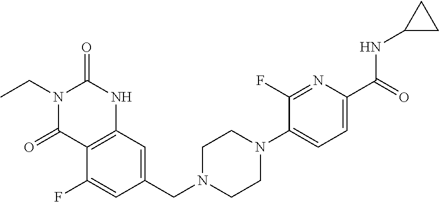 [SMILES: CCn1c(=O)[nH]c2cc(CN3CCN(c4ccc(C(=O)NC5CC5)nc4F)CC3)cc(F)c2c1=O] | 484.51 | 485.2 | DMSO-d₆: δ 11.47 (s, 1H), 8.34 (d, J = 4.7 Hz, 1H), 7.82 (d, J = 7.5 Hz, 1H), 7.55 (dd, J = 10.6, 8.1 Hz, 1H), 7.03-6.82 (m, 2H), 3.85 (q, J = 7.0 Hz, 2H), 3.55 (s, 2H), 3.21-3.07 (m, 4H), 2.87- 2.76 (m, 1H), 2.61-2.50 (m, 4H), 1.10 (t, J = 7.0 Hz, 3H), 0.67-0.57 (m, 4H). |
| 142 | 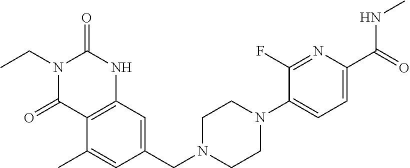 [SMILES: CCn1c(=O)[nH]c2cc(CN3CCN(c4ccc(C(=O)NC)nc4F)CC3)cc(C)c2c1=O] | 454.51 | / | / |
| 143 | 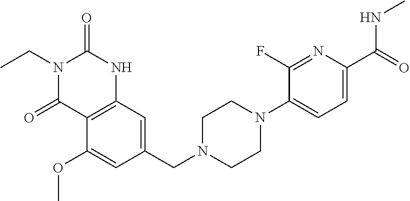 [SMILES: CCn1c(=O)[nH]c2cc(CN3CCN(c4ccc(C(=O)NC)nc4F)CC3)cc(OC)c2c1=O] | 470.51 | / | / |
| 144 | 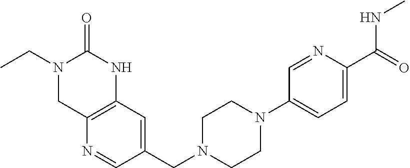 [SMILES: CCN1Cc2ncc(CN3CCN(c4ccc(C(=O)NC)nc4)CC3)cc2NC1=O] | 409.49 | 410.15 | CDCl₃ + CD₃OD: δ 7.95 (d, J = 2.5 Hz, 1H), 7.82 (s, 1H), 7.71 (d, J = 8.7 Hz, 1H), 7.03 (d, J = 8.6 Hz, 1H), 6.95 (s, 1H), 4.33 (s, 2H), 3.35 (s, 2H), 3.24 (q, J = 7.1 Hz, 2H), 3.18-3.11 (m, 4H), 2.73 (s, 3H), 2.57-2.23 (m, 4H), 0.99 (t, J = 7.2 Hz, 3H). |
| 145 | 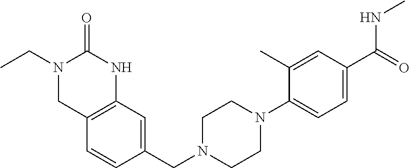 [SMILES: CCN1Cc2ccc(CN3CCN(c4ccc(C(=O)NC)cc4C)CC3)cc2NC1=O] | 421.55 | / | / |
| 146 | 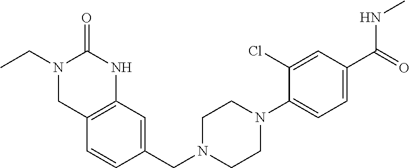 [SMILES: CCN1Cc2ccc(CN3CCN(c4ccc(C(=O)NC)cc4Cl)CC3)cc2NC1=O] | 441.96 | / | / |

### Example 147

### PARP1 and PARP2 Chemiluminescent Assay

Mix the solution of recombinant poly ADP ribotransferase 1 and 2 (PARP1 and PARP2) (40 ng enzyme/well) and the compounds to be tested, respectively. And then added to a 96-well plate coated with historic mixture, incubated at room temperature for 1 h. Then add 50 μL 0.3 ng/mL Streptavidin-HRP to each well. The plates were incubated for 30 minutes at room temperature. Finally, the plates were treated with streptavidin-HRP followed by addition of the ELISA ECL substrate to produce chemiluminescence that can then be measured using a chemiluminescence reader. The inhibition rate of the compound to PARP1/2 enzyme activity was calculated according to the following formula.

$$Inhibition ( \% ) = \frac{Readings of positive control - X}{Readings of positive control - Readings of negitive control}$$

IC₅₀ value is obtained by fitting the s-shaped dose response curve equation by using XL Fit software. The curve equation is Y=100/(1+10^(log C−log IC₅₀)), C is the compound concentration.

Table 1 summarizes the inhibitory effects of compounds on PARP1 and PARP2 enzyme activity (IC₅₀), wherein +++++ indicates IC₅₀≤1 nM; ++++ indicates 1<IC₅₀≤10 nM; +++ indicates 10 nM<IC₅₀≤100 nM: ++ indicates 100 nM<IC₅₀≤1 μM; + indicates IC₅₀>1 μM.

TABLE 1

IC₅₀, nM

| Example | PARP1 | PARP2 |
|---|---|---|
| 1 | +++++ | ++ |
| 3 | +++ | + |
| 4 | ++++ | + |
| 7 | +++++ | + |
| 10 | ++++ | + |
| 12 | ++++ | ++ |
| 13 | ++++ | ++ |
| 14 | ++++ | ++ |
| 15 | ++++ | + |
| 16 | + | + |
| 17 | ++++ | + |
| 19 | ++++ | + |
| 21 | +++++ | +++ |
| 22 | ++++ | + |
| 24 | +++ | + |
| 25 | ++++ | ++ |
| 26 | ++++ | + |
| 27 | ++++ | ++ |
| 28 | ++++ | ++ |
| 29 | ++++ | ++ |
| 30 | +++ | +++ |
| 31 | +++++ | + |
| 32 | ++++ | ++ |
| 33 | ++++ | ++ |
| 34 | ++++ | ++ |
| 40 | ++++ | + |
| 41 | ++++ | + |
| 42 | ++++ | + |
| 47 | +++ | + |
| 61 | ++++ | + |
| 62 | ++++ | ++ |
| 63 | ++++ | ++ |
| 67 | +++ | + |
| 78 | ++++ | + |
| 83 | ++++ | ++ |
| 84 | ++++ | + |
| 104 | ++++ | + |
| 114 | ++++ | + |
| 115 | ++++ | + |
| 116 | ++++ | + |
| 117 | ++++ | + |
| 119 | ++++ | + |
| 122 | ++++ | |
| 135 | ++++ | ++ |
| Senaparib | ++++ | ++++ |

Most of the compounds herein have selective inhibitory effect on PARP1 enzyme activity.

### Example 148

### Determination of the Inhibition of the Disclosed Compound on Human Breast Cancer MDA-MB-436 Cell Line

The cells were cultured in complete medium (DMEM medium +10% FBS+Insulin+glutathione). When the confluence reached about 80%, cells were digested and gently dispensed from the bottom of the dish with a 1 mL pipette. Cell suspension was collected and centrifuged at 500 rpm for 3 min. The supernatant was discarded, and the cell pellet were re-suspended in complete medium. The cells were seeded into a culture dish at an appropriate proportion, and then cultured in a 5% CO₂ incubator at 37° C. The assay was carried out when the cells were in optimum condition and the confluence was reached 80%. Cells were harvested in the logarithmic growth phase by using 1 mL pipette gently and then centrifugated at 500 rpm for 3 min. The cells were resuspended by using refresh medium after removing the supernatant and then the cells were counted. The cells were seeded at 3000/well in a 96 well plate and incubated at 37° C. 5% CO₂ incubator overnight. In the second day, cells were treated with compound at 8 serially diluted dose with 1000×final concentration in 100% DMSO. The compound was prepared as below: 1000×dilution tested compound solution to 40×test compound solution by adding 5μL, 1000×compound solution to 120 μL Medium (25-fold dilution). The solution was mixed by oscillation. DMSO was used as the control.

The medium was removed from the wells in the plate after the plate was taken out from incubator. Then, fresh medium of 195 μL/per well was added to the 96 well plate. 5μL/per well of 40×test compound solution was added into the 96 well plate. Finally, the plate was incubated for 7 days in a 37° C. 5% CO2 incubator. The medium containing compound was changed once on the fourth day. After 7 days, 20 μL of CCK-8 was added to each well and shake gently, then was cultured for 4 hours. The plate was shaken for 5 min after incubation, the absorbance values of 450 nm. and 650nm wavelengths were recorded respectively (OD=absorbance value of 450 nm−absorbance value of 650 nm) by using the multifunction readout instrument.

Data were analyzed by software GraphPad Prism 6.0. The inhibitory activity of compounds on cell proliferation was plotted using cell survival rate against the compound concentration as coordinates. Cell survival rate %=(OD_{compound}−OD_{background})/(OD_{DMSO}−OD_{background})×100. The IC₅₀ value was fitted by the s-shaped dose response curve equation: Y=100/(1+10\hat{ }(log C−log IC₅₀)), and C was the compound concentration.

Table 2 summarizes the inhibitory effect data (IC₅₀) of the compounds on the proliferation of human breast cancer cells MDA-MB-436, wherein ++++ indicates 1<IC₅₀≤10 nM; +++ indicates 10 nM<IC₅₀≤100 nM; ++ indicates 100 nM<IC₅₀≤1 μM; + indicates IC₅₀>1 μM.

TABLE 2

| Example | IC₅₀ (nM) |
|---|---|
| 1 | + |
| 2 | + |
| 3 | + |
| 4 | +++ |
| 5 | + |
| 7 | + |
| 9 | ++ |
| 10 | + |
| 12 | +++ |
| 13 | ++++ |
| 14 | +++ |
| 15 | ++ |
| 16 | + |
| 17 | +++ |
| 19 | +++ |
| 20 | ++ |
| 21 | ++++ |
| 22 | ++++ |
| 23 | ++ |
| 24 | +++ |
| 25 | ++++ |
| 26 | ++ |
| 27 | + |
| 28 | + |
| 29 | + |
| 30 | + |
| 31 | ++ |
| 32 | +++ |
| 33 | ++++ |
| 34 | +++ |
| 35 | ++ |
| 36 | + |
| 37 | + |
| 38 | + |
| 39 | + |
| 40 | + |
| 41 | + |
| 42 | + |
| 43 | + |
| 45 | + |
| 46 | ++ |
| 47 | + |
| 50 | + |
| 51 | + |
| 52 | + |
| 53 | + |
| 56 | +++ |
| 57 | + |
| 58 | + |
| 59 | +++ |
| 61 | +++ |
| 62 | +++ |
| 63 | ++++ |
| 65 | ++ |
| 66 | ++ |
| 67 | ++ |
| 68 | + |
| 69 | + |
| 70 | + |
| 71 | + |
| 72 | + |
| 74 | + |
| 78 | +++ |
| 79 | + |
| 80 | + |
| 81 | +++ |
| 82 | +++ |
| 83 | +++ |
| 84 | +++ |
| 87 | + |
| 94 | +++ |
| 95 | +++ |
| 98 | ++ |
| 99 | +++ |
| 100 | ++ |
| 101 | ++ |
| 102 | +++ |
| 103 | +++ |
| 104 | ++++ |
| 105 | ++ |
| 109 | ++ |
| 110 | + |
| 111 | + |
| 112 | + |
| 114 | +++ |
| 115 | ++++ |
| 116 | +++ |
| 117 | ++++ |
| 118 | +++ |
| 119 | ++++ |
| 120 | +++ |
| 121 | +++ |
| 122 | +++ |
| 126 | ++++ |
| 131 | ++++ |
| 132 | ++++ |
| 134 | +++ |
| 135 | ++++ |
| 136 | ++++ |
| 137 | +++ |
| 138 | +++ |
| 141 | ++++ |
| 144 | +++ |
| Senaparib | ++++ |

The compounds herein have a good inhibitory effect on the proliferation of human breast cancer cells MDA-MB-436 with BRCA mutations.

Having now fully described this disclosure, it will be understood by those of ordinary skill in the art that the same can be performed within a wide and equivalent rage of conditions, formulations and other parameters without affecting the scope of the disclosure or any embodiment thereof. All patents, patent applications and publications cited herein are fully incorporated by reference herein in their entirety.

What is claimed is:

1. A compound of Formula I: 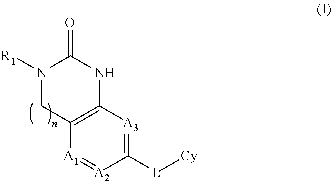
or stereoisomers, tautomers, N-oxides, hydrates, solvates, isotope-substituted derivatives, or pharmaceutically acceptable salts thereof, or mixtures thereof, or prodrugs thereof, wherein:
R₁ is selected from a group consisting of an optionally substituted alkyl, an optionally substituted carbocyclic group, an optionally substituted alkenyl and an optionally substituted alkynyl;
A₁, A₂ and A₃ are each independently selected from a group consisting of N and CR₂;
L is selected from a group consisting of bond and alkylene, optionally substituted by R₃ and/ or R₄;
Cy is selected from a group consisting of an optionally substituted heterocyclic group, an optionally substituted aryl, and an optionally substituted heteroaryl;
R₂ is selected from a group consisting of hydrogen, halogen, an optionally substituted alkyl, an optionally substituted alkoxy and an optionally substituted carbocyclic group;
R₃ and R₄ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₃ and R₄ together with the attached C form a ring;
n is selected from a group consisting of 0, 1 and 2;
wherein when n is 1 or 2, the —(CH₂)ₙ— is optionally substituted by ═O.

2. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein R₁ is a C₁₋₃ alkyl or a C₃₋₆ cycloalkyl optionally substituted by 1-5 groups selected from a group consisting of halogen, hydroxyl and —NR′R″, wherein R′ and R″ are each independently H, or C₁₋₄ alkyl or C₃₋₆ cycloalkyl optionally substituted by 1-5 groups selected from a group consisting of hydroxyl and halogen;
preferably, R₁ is C₁₋₃ alkyl, halogenated C₁₋₃ alkyl or C₃₋₄ cycloalkyl.

3. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein R₃ and R₄ are each independently halogen or C₁₋₃ alkyl, or L is a bond or an unsubstituted alkylene, preferably an unsubstituted C₁₋₃ alkylene, more preferably an unsubstituted methylene.

4. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein R₂ is hydrogen, halogen, or C₁₋₃ alkyl or C₁₋₃ alkoxy optionally substituted by 1-5 groups selected from a group consisting of halogen and hydroxyl; preferably, R₂ is hydrogen, C₁₋₃ alkyl or halogen.

5. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein:
only one of A₁, A₂ and A₃ is N, and the other two are independently CR₂, preferably, R₂ is independently H, C₁₋₃ alkyl or halogen; or
A₃ is CH, one of A₁ and A₂ is N and the other is CR₂, wherein R₂ is H, C₁₋₃ alkyl or halogen; or
A₁ is N, and both of A₂ and A₃ are CH; or
A₂ is N and both A₁ and A₃ are CH; or
all of A₁, A₂ and A₃ are CR₂, each R₂ is independently H, C₁₋₃ alkyl or halogen; or
A₃ is CH, one of A₁ and A₂ is CR₂, wherein R₂ is H, C₁₋₃ alkyl or halogen; or
both of A₂ and A₃ are CH, A1 is CR₂, wherein R₂ is C₁₋₃ alkyl or halogen.

6. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein Cy is substituted by 1-5 groups selected from a group consisting of halogen, C₁₋₄ alkyl, C₁₋₄ alkoxy, halogenated C₁₋₄ alkyl, halogenated C₁₋₄ alkoxy, an optionally substituted 6-14 membered aryl, an optionally substituted 5-10 membered heteroaryl, an optionally substituted 4-10 membered heterocyclic group, an optionally substituted C₃₋₈ cycloalkyl and —(CH₂)ₘC(O)—NRₐR_{b};
wherein Rₐ and R_{b} are each independently H, C₁₋₄ alkyl, an optionally substituted 6-14 membered aryl, an optionally substituted 5-10 membered heteroaryl or an optionally substituted 4-10 membered heterocyclic group; preferably, at least one of Rₐ and R_{b} is an optionally substituted 6-14 membered aryl, an optionally substituted 5-10 membered heteroaryl or an optionally substituted 4-10 membered heterocyclic group;
m is an integer from 0 to 5, preferably, m is 0;
wherein the said optionally substituted 6-14 membered aryl, optionally substituted 5-10 membered heteroaryl, optionally substituted 4-10 membered heterocyclic group and optionally substituted C₃₋₈ cycloalkyl are each independently optionally substituted by 1-5 groups selected from a group consisting of halogen, C₁₋₄ alkyl, C₁₋₄ alkoxy, halogenated C₁₋₄ alkyl, halogenated C₁₋₄ alkoxy, —S(O)₂—NR′R″, —NR′R″, —C(O)—NR′R″ and an optionally substituted 5-10 membered heteroaryl; preferably, the substituents on the optionally substituted 5-10 membered heteroaryl, optionally substituted 4-10 membered heterocyclic group and optionally substituted C₃₋₈ cycloalkyl in the definition of Cy and the optionally substituted 6-14 membered aryl, optionally substituted 5-10 membered heteroaryl and optionally substituted 4-10 membered heterocyclic group in the definition of Rₐ and R_{b} include at least —C(O)—NR′R″ and optionally further include any one or two of halogen, C₁₋₄ alkoxy and C₁₋₄ alkyl;
wherein the said R″ and R″ are each independently H, an optionally substituted C₁₋₄ alkyl or an optionally substituted C₃₋₆ cycloalkyl; preferably, R′ and R″ are each independently H, C₁₋₄ alkyl, halogenated C₁₋₄ alkyl or C₃₋₆ cycloalkyl;
preferably, Cy is substituted by a 5-10 membered heteroaryl, preferably by a 5-10 membered nitrogen-containing heteroaryl at least substituted by —C(O)—NR′R″, and optionally substituted by any one or two groups selected form a group consisting of halogen, C₁₋₄ alkoxy and C₁₋₄ alkyl.

7. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein Cy is substituted by R5 which is selected from a group consisting of: 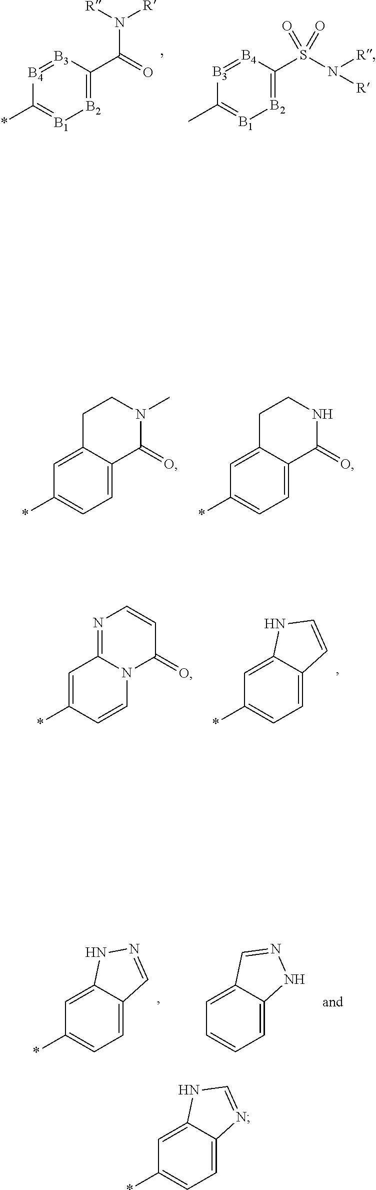
wherein B₁, B₂, B₃ and B₄ are independently selected from a group consisting of N and CR₇; R₇ is selected from a group consisting of hydrogen, halogen, C₁₋₄ alkyl, C₁₋₄ alkoxy, halogenated C₁₋₄ alkyl, halogenated C₁₋₄ alkoxy and —NR′R″; R′ and R″ are each independently selected from a group consisting of hydrogen, an optionally substituted C₁₋₄ alkyl, an optionally substituted C₃₋₆ cycloalkyl; * indicates the position at which the group is attached to the rest of the compound.

8. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein the compound of Formula I is represented by Formulae IIa and IIb as shown below: 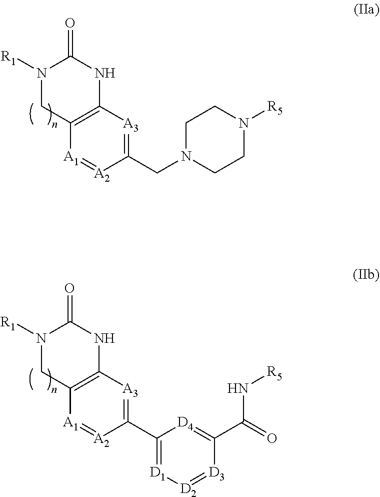
wherein:
R₁, A₁, A₂, A3 and n are as defined in any one of claims 1, 2 and 5;
R₅ is selected from a group consisting of an optionally substituted aryl and an optionally substituted heteroaryl, or is as defined in claim 6;
D₁, D₂, D₃ and D₄ are independently selected from a group consisting of N and CR₆;
R₆ is selected from a group consisting of hydrogen, halogen, an optionally substituted alkyl, an optionally substituted alkoxy, an optionally substituted carbocyclic group, an optionally substituted alkenyl, and an optionally substituted alkynyl; preferably, R₆ is hydrogen, halogen, an optionally substituted C₁₋₃ alkyl or an optionally substituted C₁₋₃ alkoxy.

9. The compound of claim 8, or stereoisomers, tautomers, N-oxides, hydrates, solvates, isotope-substituted derivatives, or pharmaceutically acceptable salts thereof, or mixtures thereof, or prodrugs thereof, wherein:
R₁ is C₁₋₃ alkyl, halogenated C₁₋₃ alkyl or C₃₋₄ cycloalkyl;
A₁, A₂ and A₃ are each independently selected from a group consisting of N and CR₂;
R₂ is hydrogen, C₁₋₃ alkyl, C₁₋₃ alkoxy or halogen;
R₅ is phenyl, pyridyl, pyrimidinyl or pyridazinyl substituted at the para position with an optionally substituted aminoacyl group; or R₅ is an optionally substituted 1-oxo-1,2,3,4-tetrahydroisoquinolin-6-yl or 4-oxo-4H-pyrido[1,2-a]pyrimidin-8-yl; or R5 is an optionally substituted pyridopyrimidinyl, indolyl, indazolyl or benzimidazolyl;
D₁, D₂, D₃ and 1D4 are independently selected from a group consisting of N and CR₆, wherein R₆ is hydrogen, C₁₋₃ alkyl, halogenated C_{1-3 ,} alkyl or halogen; and
n is 0 or 1.

10. The compound of claim 1, or stereoisomers, tautomers, N-oxides, hydrates, solvates, isotope-substituted derivatives, or pharmaceutically acceptable salts thereof, or mixtures thereof, prodrugs thereof, wherein the compound of Formula I is represented by Formulae IIIa and IIIb as shown below: 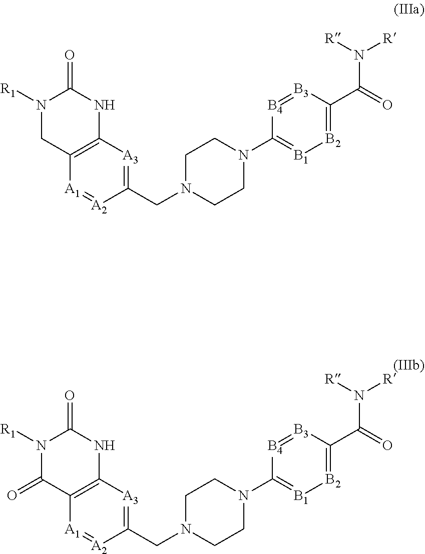
wherein:
R₁, A₁, A₂ and A₃ are as defined in any one of claims 1, 2 and 5;
B₁, B₂, B₃ and B₄ are as defined in claim 7;
R′ and R″ are each independently selected from a group consisting of hydrogen, an optionally substituted C₁₋₄ alkyl, an optionally substituted C₃₋₆ cycloalkyl, or are as defined in claim 2; or
B₃ and R″ form a 6-membered heterocyclic group with the amido to which they are attached.

11. The compound of claim 1, or stereoisomers, tautomers, N-oxides, hydrates, solvates, isotope-substituted derivatives, or pharmaceutically acceptable salts thereof, or mixtures thereof, prodrugs thereof, wherein the compound of Formula I is represented by Formulae IVa and IVb as shown below: 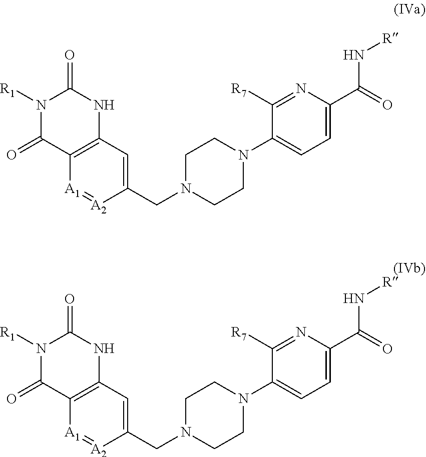
wherein:
R₁, A₁ and A₂ are as defined in any one of claims 1, 2 and 5;
R″ is as defined in claim 2;
R₇ is hydrogen, halogen, C₁₋₄ alkyl, C₁₋₄ alkoxy, halogenated C₁₋₄ alkyl, halogenated C₁₋₄ alkoxy and —NR′R″, wherein R′ and R″ are each independently selected from a group consisting of hydrogen, C₁₋₄ alkyl, halogenated C₁₋₄ alkyl or C₃₋₆ cycloalkyl.

12. The compound of claim 1, wherein the compound is selected from a group consisting of a group consisting of:
5-(4-((3-ethyl-2,4-dioxo-1,2,3,4-tetrahydropyrido[3,2-d]pyrimidin-7-yl)methyl)piperazin-1-yl)-N-methylpicolinamide;
5-(4-((3-methyl-2,4-dioxo-1,2,,1,4-tetrahydropyrido[3,2-d]pyrimidin)7-methyl)piperazin-1-yl)-N-methylpicolinamide;
5-(4-((3-isopropyl-2,4-dioxo-1,2,3,4-tetrahydropyrido[3,2-d]pyrimidin-7-yl)methyl)piperazin-1-yl)-N-methylpicolinamide;
5-(4-((3-ethyl-2,4-dioxo-1,2,3,4-tetrahydropyrido[3,2-d]pyrimidin-7-yl)methyl)piperazin-1-yl)-N,6-dimethylpicolinamide;
5-(4-((3-ethyl-2,4-dioxo-1,2,3,4-tetrahydropyrido[4,3-d]pyrimidin-7-yl)methyl)piperazin-1-yl)-N-methylpicolinamide;
5-(4-((3-ethyl-6-fluoro-2,4-dioxo-1,2,3,4-tetrahydropyrido[3,2-d]pyrimidin-7-yl)methyl)piperazin-1-yl)-N-methylpicolinamide;
5-(4-((2,4-dioxo-3-propyl-1,2,3,4-tetrahydropyrido[3,2-d]pyrimidin-7-yl)methyl)piperazin-1-yl)-N-methylpicolinamide;
5-(4-((2,4-dioxo-3-(trifluoromethyl)-1,2,3,4-tetrahydropyrido[3,2-d]pyrimidin-7-yl)methyl)piperazin-1-yl)-N-methylpicolinamide;
5-(4-((3-ethyl-2,4-dioxo-1,2,3,4-tetrahydropyrido[3,2-d]pyrimidin-7-yl)methyl)piperazin-1-yl)-6-fluoro-N-methylpicolinamide;
5-(4-((3-ethyl-2,4-dioxo-1,2,3,4-tetrahydropyrido[3,2-d]pyrimidin-7-yl)methyl)piperazin-1-yl)-6-chloro-N-methylpicolinamide;
5-(4-((3-ethyl-2,4-dioxo-1,2,3,4-tetrahydropyrido[3,2-d]pyrimidin-7-yl)methyl)piperazin-1-yl)-N-methyl-6-(trifluoromethyl)picolinamide;
5-(4-((3-ethyl-2,4-dioxo-1,2,3,4-tetrahydroquinazolin-7-yl)methyl)piperazin-1-yl)-N-methylpicolinamide;
5-(4-((3-ethyl-2,4-dioxo-1,2,3,4-tetrahydroquinazolin-7-yl)methyl)piperazin-1-yl)-6-fluoro-N-methylpicolinamide;
5-(4-((3-ethyl-6-fluoro-2,4-dioxo-1,2,3,4-tetrahydroquinazolin-7-yl)methyl)piperazin-1-yl)-6-fluoro-N-methylpicolinamide;
5-(4-((3-ethyl-2-oxo-1,2,3,4-tetrahydroquinazolin-7-yl)methyl)piperazin-1-yl)-N-methylpicolinamde;
5-(4-((1-ethyl-2-oxo-2,3-dihydro-1H-benzo[d]yl)methyl)piperazin-1-yl)-N-methylpicolinamide;
5-(4-((3-methyl-2,4-dioxo-1,2,3,4-tetrahydroquinazolin-7-yl)methyl)piperazin-1-yl)-6-fluoro-N-methylpicolinamide;
5-(4-((2,4-dioxo-3-(trifluoromethyl)-1,2,3,4-tetrahydroquinazolin-7-yl)methyl)piperazin-1-yl)-6-fluoro-N-methylpicolinamide;
5-(4-((2,4-dioxo-3-propyl-1,2,3,4-tetrahydroquinazolin-7-yl)methyl)piperazin-1-yl)-6-fluoro-N-methylpicolinamicle;
5-(4-((3-(2-fluoroethyl)-2,4-dioxo-1,2,3,4-tetrahydroquinazolin-7-yl)methyl)piperazin-1-yl)- N,6-dimethylpicolinamide;
5-(4-((3-ethyl-2,4-dioxo-1,2,3,4-tetrahydroquinazolin-7-yl)methyl)piperazin-yl)-6-chloro-N-methylpicolinamide;
5-(4-((3-ethyl-2,4-dioxo-1,2,3,4-tetrahydroquinazolin-7-yl)methyl)piperazin-1-yl)-N,6-dimethylpicolinamide;
5-(4-((3-ethyl-2,4-dioxo-1,2,3,4-tetrahydroquinazolin-7-yl)methyl)piperazin-1-yl)-N-methyl-6-(trifluoromethyl)picolinamide;
5-(4-((3-ethyl-8-fluoro-2,4-dioxo-1,2,3,4-tetrahydroquinazolin-7-yl)methyl)piperazin-1-yl)-6-fluoro-N-methylpicolinanamide;
5-(4-((3-ethyl-5-fluoro-2,4-dioxo-1,2,3,4-tetrahydroquinazolin-7-yl)methyl)piperazin-1-yl)-6-fluoro-N-methylpicolinamide;
5-(4-((3-isopropyl-2,4-dioxo-1,2,3,4-tetrahydroquinazolin-7-yl)methyl)piperazin-1 yl)-6-fluoro-N-methylpicolinamide;
5-(4-((3-ethyl-2,4-dioxo-1,2,3,4-tetrahydroquinazolin-7-yl)methyl)piperazin- l-yl)-N-methylpyrimidine-2-carboxamide;
6-(4-((3-ethyl-2,4-dioxo-1,2,3,4-tetrahydroquinazolin-7-yl)methyl)piperazin-1-yl)-N-methylnicotinamide;
6-(4-((3-ethyl-2,4-dioxo-1,2,3,4-tetrahydroquinazolin-7-yl)methyl)piperazin-1-yl)-N-methylpyridazine-3-carboxamide;
2-(4-((3-ethyl-2,4-dioxo-1,2,3,4-tetrahydroquinazolin-7-yl)methyl)piperazin-1-yl)-N-methylpyrimidine-5-carboxamide;
5-(4-((3-ethyl-2,4-dioxo-1,2,3,4-tetrahydroquinazolin-7-yl)methyl)piperazin-1-yl)-6-ethyl-N-methylpicolinamide;
5-(4-((3-ethyl-2,4-dioxo-1,2,3,4-tetrahydroquinazolin-7-yl)methyl)piperazin-1-yl)-6-fluoropicolinamide;
5-(4-((3-ethyl-2,4-dioxo-1,2,3,4-tetrahydroquinazolin-7-yl)methyl)piperazin-1-yl)-6-fluoro-N-ethylpicolinamide;
5-(4-((3-ethyl-2,4-dioxo-1,2,3,4-tetrahydroquinazolin-7-yl)methyl)piperazin-1-yl)-6-fluoro-N-isopropylpicolinamide;
5-(4-((3-ethyl-2,4-dioxo-1,2,3,4-tetrahydroquinazolin-7-yl)methyl)piperazin-1-yl)-6-(difluoromethyl)-N-methylpicolinamide;
3-ethyl-7-((4-(2-methyl-1-oxo-1,2,3,4-tetrahydroisoquinolin-6-yl)piperazin-1-yl)methyl)quinazoline-2,4(1H,3H)-dione;
3-ethyl-7-((4-(1-oxo-1,2,3,4-tetrahydroisoquinolin-6-yl)piperazin-1-yl)methyl)quinazoline-2,4(1H,3H)-dione;
3-ethyl-7-((4-(4-oxo-4H-pyrido[1 ,2-a]pyrimidin-8-yl)piperazin-1-yl)methyl)quinazoline-2,4(1H,3 H)-dione;
5-(4-((3-ethyl-2,4-dioxo-1,2,3,4-tetrahydroquinazolin-7-yl)methyl)piperazin-1-yl)-6-fluoro-N,N-dimethylpicolinamide;
5-(3-(3-methyl-2,4-dioxo-1,2,3,4-tetrahydroquinazolin-7-yl)benzamido)N-methylpicolinamide;
5-(3-(3-ethyl-2,4-dioxo-1,2,3,4-tetrahydroquinazolin-7-yl)benzamido)-N-methylpicolinamide;
5-(3-(3-isopropyl-2,4-dioxo-1,2,3,4-tetrahydroquinazolin-7-yl)benzamido)-N-methylpicolinamide;
5-(3-(3-ethyl-2,4-dioxo-1,2,3,4-tetrahydroquinazolin-7-yl)-5-fluorobenzamido)-N-methylpicolinamide;
5-(3-(3-ethyl-2,4-dioxo-1,2,3,4-tetrahydroquinazolin-7-yl)benzamido)-6-fluoro-N-methylpicolinamide;
5-(3-(3-ethyl-2,4-dioxo-1,2,3,4-tetrahydropyrido[3,2-d]pyrimidin-7-yl)benzamido)-N-methylpicolinamide;
6-(3-ethyl-2,4-dioxo-1,2,3,1-tetrahydroquinazolin-7-yl)-N-(6-(methylcarbamoyl)pyridin-3-yl)picolinamide;
5-(3-(3-propyl-2,4-dioxo-1,2,3,4-tetrahydroquinazolin-7-yl)benzamido)-N-methylpicolinamide;
5-(4-((3-ethyl-2,4-dioxo-1,2,3,4-tetrahydroquinazolin-7-yl)methyl)piperazin-1-yl)-4-fluoro-N-methylpicolinamide;
5-(4-((3-ethyl-2,4-dioxo-1,2,3,4-tetrahydroquinazolin-7-yl)methyl)piperazin-1-yl,-3-fluoro-N-methylpicolinamide;
5-(4-((3-ethyl-2,4-dioxo-1,2,3,4-tetrahydroquinazolin-7-yl)methyl)piperazin-1)-N,4-dimethylpicolinamide;
5-(4-((3-ethyl-2,4-dioxo-1,2,3,4-tetrahydroquinazolin-7-yl)methyl)piperazin-1-yl)-N,3-dimethylpicolinamide;
5-(4-((3-ethyl-6-methyl-2,4-dioxo-1,2,3,4-tetrahydroquinazolin-7-yl)methyl)piperazin-1-yl)-N,6-dimethylpicolinamide;
5-(4-((6-chloro-3-ethyl-2,4-dioxo-1,2,3,4-tetrahydroquinazolin-7-yl)methyl)piperazin-1-yl)-N,6-dimethylpicolinamide;
3-ethyl-7-((4-(2-methyl-6-(5-methyl-1,3,4-oxadiazol-2-yl)pyridin-3-yl)piperazin-1-yl)methyl)quinazoline-2,4(1H,3H)-dione;
5-(4-((3-ethyl-2,4-dioxo-1,2,3,4-tetrahydroquinazolin-7-yl)methyl)piperazin-1-yl)-N,6-dimethylpyridine-2-sulfonamide;
5-(4-((3-ethyl-5-fluoro-2,4-dioxo-1,2,3,4-tetrahydroquinazolin-7-yl)methyl)piperazin-1-yl)-N,6-dimethylpicolinamide;
5-(4-((3-ethyl-2,4-dioxo-1,2,3,4-tetrahydroquinazolin-7-yl)methyl)piperazin-1-yl)-N-methylpyrazine-2-carboxamide;
5-(1-((3-ethyl-2,4-dioxo-1,2,3,4-tetrahydroquinazolin-7-yl)methyl)piperidin-4-yl)-N,6-(dimethylpicolinamide;
5-(4-((5-chloro-3-ethyl-2,4-dioxo-1,2,3,4-tetrahydroquinazolin-7-yl)methyl)piperazin-1-yl)-N,6-dimethylpicolinamide;
5-(4-((3-ethyl-5-methyl-2,4-dioxo-1,2,3,4-tetrahydroquinazolin-7-yl)methyl)piperazin-1-yl)-N,6-dimethylpicolinamide;
6-chloro-5-(4-((3-ethyl-6-fluoro-2,4-dioxo-1,2,3,4-tetrahydroquinazolin-7-methyl)piperazin-1-yl)-N-methylpicolinamide;
6-chloro-5-(4-((3-ethyl-5-fluoro-2,4-dioxo-1,2,3,4-tetrahydroquinazolin-7-methyl)piperazin-1-yl1)-N-methylpicolinamide;
6-chloro-5-(4-((5-chloro-3-ethyl-2,4-dioxo-1,2,3,4-tetrahydroquinazolin-7-yl)methyl)piperazin-1-yl)-N-methylpicolinamide;
6-chloro-5-(4-((3-ethyl-5-methyl-2,4-dioxo-1,2,3,4-tetrahydroquinazolin-7-yl)methyl)piperazin-1-yl)-N-methylpicolinamide;
6-chloro-5-(4-((6-chloro-3-ethyl-2,4-dioxo-1,2,3,4-tetrahydroquinazolin-7-yl)methyl)piperazin-1-yl)-N-methylpicolinamide;
6-chloro-5-(4-((3-ethyl-6-methyl-2,4-dioxo-1,2,3,4-tetrahydroquinazolin-7-yl)methyl)piperazin-1-yl)-N-methylpicolinamide;
5-(4-((3-ethyl-6-fluoro-2,4-dioxo-1,2,3,4-tetrahydroquinazolin-7-yl)methyl)piperazin-1-yl)-N,6-dimethylpicolinamide;
4-(4-((3-ethyl-2,4-dioxo-1,2,3,4-tetrahydroquinazolin-7-yl)methyl)piperazin-1-yl)-N-methylbenzamide;
4-(4-((3-ethyl-2,4-dioxo-1,2,3,4-tetrahydroquinazolin-7-yl)methyl)piperazin-1-yl,-3-fluoro-N-methylbenzamide;
3-chloro-4-(4-((3-ethyl-2,4-dioxo-1,2,3,4-tetrahydroquinazolin-7-yl)methyl)piperazin-1-yl)-N-methylbenzamide;
4-(4-((3-ethyl-2,4-dioxo-1,2,3,4-tetrahydroquinazolin-7-yl)methyl)piperazin-1-yl)-N,3-dimethylbenzamide;
5-(4-((3-ethyl-2,4-dioxo-1,2,3,4-tetrahydroquinazolin-7-yl)methyl)piperazin-1-yl)-N,4,6-trimethylpicolinamide;
4-chloro-5-(4-((3-ethyl-2,4-dioxo-1,2,3,4-tetrahydroquinazolin-7-yl)methyl)piperazin-1-yl)-N,6-dimethylpicolinamide;
5-(4-((3-ethyl-2,4-dioxo-1,2,3,4-tetrahydropyrido[2,3-d]pyrimidin-7-yl)methyl)piperazin-1-yl)-N,6-dimethylpicolinamide;
5-(4-((3-ethyl-2,4-dioxo-1,2,3,4-tetrahydroquinazolin-7-yl)methyl)piperazin-1-yl)-N,4-dimethylpyrimidine-2-carboxamide;
5-(4-((3-ethyl-2,4-dioxo-1,2,3,4-tetrahydroquinazolin-7-yl)methyl)piperazin-1-yl)-N,6-dimethylpyrazine-2-carboxamide;
6-(4-((3-ethyl-2,4-dioxo-1,2,3,4-tetrahydroquinazolin-7-yl)methyl)piperazin-1-yl)-N,5-dimethylpyridazine-3-carboxamide;
N-cyclopropyl-5-(4-((3-ethyl-2,4-dioxo-1,2,3 ,4-tetrahydroquinazolin-7-yl)methyl)piperazin-1-yl)-6-methylpicolinamide;
6-(4-((3-ethyl-2,4-dioxo-1,2,3,4-tetrahydroquinazolin-7-yl)methyl)piperazin-1-yl)-N,5-dimethylnicotinamide;
5-(4-((3-ethyl-2,4-dioxo-1,2,3,4-tetrahydroquinazolin-7-yl)methyl)piperazin-1-yl)-6-isopropyl-N-methylpicolinamide;
5-(4-((3-ethyl-2,4-dioxo-1,2,3,4-tetrahydropyrido[4,3-A]pyrimidin-7-yl)methyl)piperazin-1-yl)-N,6-dimethylpicolinamide;
N-ethyl-5-(4-((3-ethyl-5-fluoro-2,4-dioxo-1,2,3,4-tetrahydroquinazolin-7-yl)methyl)piperazin-1-yl)-6-methylpicolinamide;
6-chloro -N-ethyl-5-(4-((3-ethyl-2,4-dioxo-1,2,3,4-tetrahydroquinazolin-7-yl)methyl)piperazin-1-yl)picolinamide;
N-ethyl-5-(4-((3-ethyl-2,4-dioxo-1,2,3,4-tetrahydroquinazolin-7-yl)methyl)piperazin-1-yl)-6-methylpicolinamide;
N,6-dimethyl-5-(4-((3-methyl-2,4-dioxo-1,2,3,4-tetrahydropyrido[3,2-d]pyrimidin-7-methyl)piperazin-1-yl)picolinamide;
6-chloro-N-methyl-5-(4-((3-methyl-2,4-dioxo-1,2,3,4-tetrahydropyrido[3,2-d]pyrimidin-7-yl)methyl)piperazin-1-yl)picolinamide;
5-(4-((2,4-dioxo-3-(2,2,2-trifluoroethyl)-1,2,3,4-tetrahydroquinazolin-7-yl)methyl)piperazin-1-yl)-N,6-dimethylpicolinamide;
5-(4-((3-ethyl-2,4-dioxo-1,2,3,4-tetrahydroquinazolin-7-yl)methyl)piperazin-1-yl)-4-fluoro-N,6-dimethylpicolinamide;
5-(4-((3-ethyl-2,4-dioxo-1,2,3,4-tetrahydroquinazolin-7-yl)methyl)piperazin-1-yl)-N,3,6-trimethylpicolinamide;
5-(4-((3-ethyl-2,4-dioxo-1,2,3,4-tetrahydroquinazolin-7-yl)methyl)piperazin-1-yl)-3-fluoro-N,6-dimethylpicolinamide;
3-chloro-5-(4-((3-ethyl-2,4-dioxo-1,2,3,4-tetrahydroquinazolin-7-yl)methyl)piperazin-1-yl)-N,6-dimethylpicolinamide;
6-chloro-5-(4-((3-ethyl-2,4-dioxo-1,2,3,4-tetrahydroquinazolin-7-yl)methyl)piperazin-1-yl)-N,3-dimethylpicolinamide;
6-chloro-5-(4-((3-ethyl-2,4-dioxo-1,2,3,4-tetrahydroquinazolin-7-yl)methyl)piperazin-1-yl)-N,4-dimethylpicolinamide;
6-bromo-5-(4-((3-ethyl-2,4-dioxo-1,2,3,4-tetrahydroquinazolin-7-yl)methyl)piperazin-1-yl)-N-methylpicolinamide;
6-bromo-N-ethyl-5-(4-((3-ethyl-2,4-dioxo-1,2,3,4-tetrahydroquinazolin-7-yl)methyl)piperazin-1-yl)picolinamide;
N-ethyl-5-(4-((3-ethyl-2,4-dioxo-1,2,3,4-tetrahydroquinazolin-7-yl)methyl)piperazin-1-yl)-6-(trifluoromethyl)picolinamide;
5-(4-((3-ethyl-2,4-dioxo-1,2,3,4-tetrahydroquinazolin-7-yl)methyl)piperazin-1-yl)-6-methyl-N-(trifluoromethyl)picolinamide;
6-chloro-N-methyl-5-(4-((3-methyl-2,4-dioxo-1,2,3,4-tetrahydroquinazolin-7-yl)methyl)piperazin-1-yl)picolinamide;
N,6-dimethyl-5-(4-((3-methyl-2,4-dioxo-1,2,3,4-tetrahydroquinazolin-7-yl)methyl)piperazin-1-yl)picolinamide;
6-chloro-N-ethyl-5-(4-((3-methyl-2,4-dioxo-1,2,3,4-tetrahydroquinazolin-7-yl)methyl)piperazin-1-yl)picolinamide;
N-ethyl-6-methyl-5-(4-((3-methyl-2,4-dioxo-1,2,3,4-tetrahydroquinazolin-7-yl)methyl)piperazin-1-yl)picolinamide;
6-chloro-N-ethyl-5-(4-((3-ethyl-2,4-dioxo-1,2,3,4-tetrahydropyrido[3,2-d]pyrimidin-7-yl)methyl)piperazin-1-yl)picolinamide;
N-ethyl-5-(4-((3-ethyl-2,4-dioxo-1,2,3,4-tetrahydropyrido[3,2-d]pyrimidin-7-yl)methyl)piperazin-1-yl)-6-methylpicolinamide;
6-chloro-N-ethyl-5-4-((3-ethyl-5-fluoro-2,4-dioxo-1,2,3,4-tetrahydroquinazolin-7-yl)methyl)piperazin-1-yl)picolinamide;
5-(4-((5-fluoro-3-methyl-2,4-dioxo-1,2,3,4-tetrahydroquinazolin-7-y)methyl)piperazin-1-yl)-N,6-dimethylpicolinamide;
6-chloro-5-(4-((5-fluoro-3-methyl-2,4-dioxo-1,2,3,4-tetrahydroquinazolin-7-yl)methyl)piperazin-1-yl)-N-methylpicolinamide;
N-ethyl-5-(4-((5-fluoro-3-methyl-2,4-dioxo-1,2,3,4-tetrahydroquinazolin-7-yl)methyl)piperazin-1-yl)-6-methylpicolinamide;
6-chloro-N-ethyl-5-(4-((5-fluoro-3-methyl-2,4-dioxo-1,2,3,4-tetrahydroquinazolin-7-yl)methyl)piperazin-1-yl)picolinamide;
5-(4-((3-ethyl-2,4-dioxo-1,2,3,4-tetrahydropyrido[3,2-d]pyrimidin-7-yl)methyl)piperazin-1-yl)-6-methoxy-N-methylpicolinamide;
7-((4-(1H-indol-6-yl)piperazin-1-yl)methyl)-3-ethylquinazoline-2,4(1H,3H)-dione;
7-((4-(1H-indazol-6-yl)piperazin-1-yl)methyl)-3-ethylquinazoline-2,4(1H ,3H)-dione;
7-((4-(1H-indazol-5-yl)piperazin-1-yl)methyl)-3-ethylquinazoline-2,4(1H ,3H)-dione;
7-((4-(1H-benzo[d]imidazol-6-yl)piperazin-1-yl)methyl)-3-ethylquinazoline-2,4(1H ,3H)-dione;
5-(4-((3-ethyl-2-oxo-1,2,3,4-tetrahydroquinazolin-7-yl)methyl)piperazin-1-yl)-6-fluoro-N-methylpicolinamde;
6-chloro-5-(4-((3-ethyl-2-oxo-1,2,3,4-tetrahydroquinazolin-7-yl)methyl)piperazin-1-yl)-N-methylpicolinamide;
5-(4-((3-ethyl-2-oxo-1,2,3,4-tetrahydroquinazolin-7-yl)methyl)piperazin-1-yl)-N,6-dimethylpicolinamide;
5-(4-((3-ethyl-2-oxo-1,2,3,4-tetrahydroquinazolin-7-yl)methyl)piperazin-1-yl)-6-methyl-N-ethylpicolinamide;
5-(4-((3-ethyl-2-oxo 1,2,3,4-tetrahydroquinazolin-7-yl)methyl)piperazin-1-yl)-6-chloro-N-ethylpicolinamide;
5-(4-((3-ethyl-5-fluoro-2-oxo-1,2,3,4-tetrahydroquinazolin-7-yl)methyl)piperazin-1-yl)-N,6-dimethylpicolinamide;
6-chloro-5-(4-((3-ethyl-5-fluoro-2-oxo-1,2,3,4-tetrahydroquinazolin-7-yl)methyl)piperazin-1-yl)-N-methylpicolinamide;
N-ethyl-5-(4-((3-ethyl-5-fluoro-2-oxo-1,2,3,4-tetrahydroquinazolin-7-yl)methyl)piperazin-1-yl)-6-methylpicolinamide;
6-chloro-N-ethyl-5-(4-((3-ethyl-5-fluoro-2-oxo-1,2,3,4-tetrahydroquinazolin-7-yl)methyl)piperazin-1-yl)picolinamide;
5-(4-((3-ethyl-5-methoxy-2,4-dioxo-1,2,3,4-tetrahydroquinazolin-7-yl)methyl)piperazin-1-yl)-N,6-dimethylpicolinamide;
6-chloro-5-(4-((3-ethyl-5-methoxy-2,4-dioxo-1,2,3,4-tetrahydroquinazolin-7-yl)methyl)piperazin-1-yl)-N-methylpicolinamide;
5-(4-((5-chloro-3-ethyl-2,4-dioxo-1,2,3,4-tetrahydroquinazolin-7-yl)methyl)piperazin-1yl)-N-ethyl-6-methylpicolinamide;
6-chloro-5-(4-((5-chloro-3-ethyl-2,4-dioxo-1,2,3,4-tetrahydroquinazolin-7-yl)methyl)piperazin-1-yl)-N-ethylpicolinamide;
5-(4-((5-chloro-3-ethyl-2-oxo-1,2,3,4-tetrahydroquinazolin-7-yl)methyl))piperazin-1-yl)-N,6-dimethylpicolinamide;
6-chloro-5-(4-((5-chloro-3-ethyl-2-oxo-1,2,3,4-tetrahydroquinazolin-7-yl)methyl)piperazin-1-yl)-N-methylpicolinamide;
5-(4-((5-chloro-3-ethyl-2-oxo-1,2,3,4-tetrahydroquinazolin-7-yl)methyl)piperazin-1-yl)-N-ethyl-6-methylpicolinamide;
6-chloro-5-(4-((5-chloro-3-ethyl-2-oxo-1,2,3,4-tetrahydroquinazolin-7-yl)methyl)piperazin-1-yl)-N-ethylpicolinamide;
6-chloro-5-(4-((5-chloro-3-ethyl-2,4-dioxo-1,2,3,4-tetrahydroquinazolin-7-yl)methyl)piperazin-1-yl)-N-cyclopropylpicolinamide;
6-chloro-N-cyclopropyl-5-(4-((3-ethyl-2-oxo-1,2,3,4-tetrahydroquinazolin-7-yl)meth)piperazin-1-yl)picolinamide;
N-ethyl-5-(4-((3-ethyl-2-oxo-1,2,3,4-tetrahydroquinazolin-7-yl)methyl)piperazin-1-yl)-6-fluoropicolinamide;
5-(4-((3-ethyl-5-fluoro-2-oxo-1,2,3,4-tetrahydroquinazolin-7-yl)methyl)piperazin-1-yl)-6-fluoro-N-methylpicolinamide;
N-cyclopropyl-5-(4-((3-ethyl-2,4-dioxo-1,2,3,4-tetrahydroquinazolin-7-yl)methyl)piperazin-1-yl)-6-fluoropicolinamide;
5-(4-((5-chloro-3-ethyl-2,4-dioxo-1,2,3,4-tetrahydroquinazolin-7-yl)methyl)piperazin-1-yl)-6-fluoro-N-methylpicolinamide;
N-cyclopropyl-5-(4-((3-ethyl-2-oxo-1,2,3,4-tetrahydroquinazolin-7-yl)methyl)piperazin-yl)-6-methylpicolinamide;
6-fluoro-5-(4-((8-fluoro-3-methyl-2,4 dioxo-1,2,3,4-tetrahydroquinazolin-7-yl)methyl)piperazin-1-yl)-N-methylpicolinamide;
6-fluoro-5-(4-((8-fluoro-3-methyl-2-oxo-1,2,3,4-tetrahydroquinazolin-7-yl)methyl)piperazin-1-yl)-N-methylpicolinamide;
5-(4-((3-cyclopropyl-2,4-dioxo-1,2,3,4-tetrahydroquinazolin-7-yl)methyl)piperazim-1-yl)-6-fluoro-N-methylpicolinamide;
N -cyclopropyl-5-(4-((3-ethyl-5-fluoro-2,4-dioxo-1,2,3,4-tetrahydroquinazolin-7-yl)methyl)piperazin-1-yl)-6-fluoropicolinamide;
5-(4-((3-ethyl-5-methyl-2,4-dioxo-1,2,3,4-tetrahydroquinazolin-7-yl)methyl)piperazin-1-yl)-6-fluoro-N-methylpicolinamide;
5-(4-((3-ethyl-5-methoxy-2,4-dioxo-1,2,3,4-tetrahydroquinazolin-7-yl)methyl)piperazin-1-yl)-6-fluoro-N-methylpicolinamide;
5-(4-((3-ethyl-2-oxo-1,2,3,4-tetrahydropyrido[3,2-d]pyrimidin-7-yl)methyl)piperazin-1-yl)-N-methylpicolinamide;
4-(4-((3-ethyl-2-oxo-1,2,3,4-tetrahydroquinazolin-7-yl)methyl)piperazin-1-yl)-N,3-dimethylbenzamide;
3-chloro-4-(4-((3-ethyl-2-oxo-1,2,3,4-tetrahydroquinazolin-7-yl)methyl)piperazin-1-yl)-N-methylbenzamide;
or stereoisomers, tautomers, N-oxides, hydrates, isotope-substituted derivatives, solvates or pharmaceutically acceptable salts thereof, or mixtures thereof, or prodrugs thereof.

13. Use of the compound of any one of claims 1-12, or stereoisomers, tautomers, N-oxides, hydrates, isotope-substituted derivatives, solvates or pharmaceutically acceptable salts thereof, or mixtures thereof, or prodrugs thereof in the manufacture of a medicament for treatment or prevention of a disease or condition responsive to the inhibition of PARP activity; preferably, the disease or condition is cancer; preferably, the cancer is liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoide, head and neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma;
preferably, the medicament further comprises at least one known anticancer drug or a pharmaceutically acceptable salt thereof; preferably, the anticancer drug is selected from a group consisting of a group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, ostinib, afatinib, ceritinib, alectinib, crizotinib, erlotinib, lapatinib, solutinib lafenib, regorafenib, vemurafenib, dabrafenib, aflibercept, sunitinib, nilotinib, dasatinib, bosutinib, pratinib, brutinib, cabozantinib, lenvatinib, vandetanib, trametinib, cabitinib, axitinib, temsirolimus, idelalisib, pazopanib, everolimus, tamoxifen, letrozole, fulvestrant, mitoguanhydrazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine);
preferably, the medicament is used in combination with radiotherapy.

14. A pharmaceutical composition comprising the compound of any one of claims 1-12, or stereoisomers, tautomers, N-oxides, hydrates, isotope-substituted derivatives, solvates or pharmaceutically acceptable salts thereof, or mixtures thereof and a pharmaceutically acceptable carrier.

15. The pharmaceutical composition of claim 14, wherein the composition further includes at least one known anticancer drug or pharmaceutically acceptable salts thereof; preferably, the at least one known anticancer drug is selected from a group consisting of the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, ostinib, afatinib, ceritinib, alectinib, crizotinib, erlotinib, lapatinib, solutinib lafenib, regorafenib, vemurafenib, dabrafenib, aflibercept, sunitinib, nilotinib, dasatinib, bosutinib, pratinib, brutinib, cabozantinib, lenvatinib, vandetanib, trametinib, cabitinib, axitinib, temsirolimus, idelalisib, pazopanib, everolimus, tamoxifen, letrozole, fulvestrant, mitoguanhydrazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine).
